(19) United States
(12) Patent Application Publication
(10) Pub. No.: US 2024/0197738 A1
(43) Pub. Date: Jun. 20, 2024

(54) COMPOUND 7AI IN TREATING EWING SARCOMA BY INHIBITING OTUD7A

(71) Applicants: The University of North Carolina at Chapel Hill, Chapel Hill, NC (US); Atomwise Inc., San Francisco, CA (US)

(72) Inventors: Pengda Liu, Chapel Hill, NC (US); Siyuan Su, Chapel Hill, NC (US); Ian Jonathan Davis, Chapel Hill, NC (US); Christian Laggner, San Franciso, CA (US)

(21) Appl. No.: 18/284,416

(22) PCT Filed: Apr. 5, 2022
(86) PCT No.: PCT/US2022/023484

§ 371 Date: Sep. 27, 2023

Related U.S. Application Data
(60) Provisional application No. 63/170,977, filed on Apr. 5, 2021.

(51) Int. Cl. A61K 31/519; A61P 35/00

(52) U.S. Cl. CPC A61K 31/519; A61P 35/00

(57) ABSTRACT

Disclosed are compositions for targeting 0TUD7A, the compositions having a component sufficient to block and/or reduce OTUD7A-mediated deubiquitination of EWS-FLI1 in a cell. The component can be 7Ai and variants thereof. The compositions can be used to treat Ewing sarcoma (EWS). Methods of using the disclosed compositions are also disclosed, including methods of treating EWS in a subject.t

### CROSS-REFERENCE TO RELATED APPLICATIONS

This application claims priority to U.S. Patent Application Ser. No. 63/170,977, filed on Apr. 5, 2021, the disclosure of which is incorporated by reference herein in its entirety.

### GRANT STATEMENT

This invention was made with government support under Grant No. CA234979 awarded by the NIH. The government has certain rights in the invention.

### TECHNICAL FIELD

The presently disclosed subject matter is directed to methods and compositions for treating Ewing sarcoma (EWS). More particularly, the presently disclosed subject matter is directed to compound 7ai in treating EWS by inhibiting OTUD7A.

### BACKGROUND

Ewing sarcoma (EWS) is an aggressive malignancy that develops in bones or soft tissues of children and young adults. There is a need for new and improved treatments and therapies for EWS.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

Provided in some embodiments are compositions for targeting OTUD7A, the compositions comprising a component sufficient to block and/or reduce OTUD7A-mediated deubiquitination of EWS-FLI1 in a cell, wherein the component comprises 7Ai and variants thereof. In some embodiments, the component is a catalytic inhibitor, wherein the catalytic inhibitor inhibits the catalytic activity of OTUD7A. In some embodiments, blocking and/or reducing OTUD7A-mediated deubiquitination of EWS-FLI1 substantially destabilizes EWS-FLI1, optionally wherein the stability of EWS-FLI1 is reduced by about 10% to about 90%, or about 20% to about 80%, or about 30% to about 70%, or about 50% or more. In some embodiments, the component substantially reduces EWS-FLI1 levels in the cell. In some embodiments, the cell is a human cell, optionally wherein the human cell is in vivo. In some embodiments, the compositions are configured for in vivo administration to a subject.

In some embodiments, 7Ai comprises the following structure:

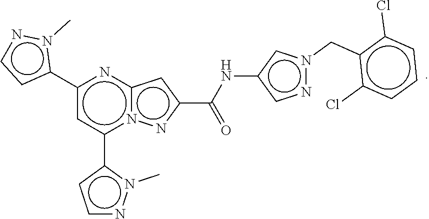

In some embodiments, 7Ai is in the compositions at a concentration sufficient to provide a dosage of about 10 μM when administered to a subject, optionally about 1 μM to about 20 μM. In some embodiments, the compositions comprise a delivery vehicle for the component, optionally wherein the delivery vehicle is an expression vector, nanoparticle, liposome or vesicle. In some embodiments, the compositions are configured for treating a cancer, optionally wherein the cancer is Ewing sarcoma.

Also disclosed herein in some embodiments are OTUD7A catalytic inhibitors, the catalytic inhibitors comprising a 7Ai compound or variant thereof. In some embodiments, the catalytic inhibitors are configured to substantially limit Ewing sarcoma growth in vivo by degrading EWS-FLI1. In some embodiments, the 7Ai compound in the catalytic inhibitors comprises the following structure:

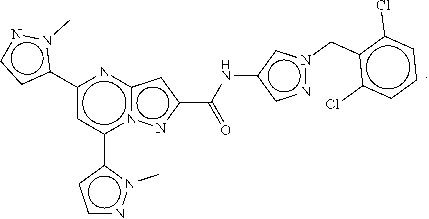

Provided herein in some aspects are methods of treating Ewing sarcoma and related conditions, the method comprising administering to a subject having Ewing sarcoma or suspected of suffering from Ewing sarcoma a composition comprising a component targeting a fusion oncoprotein, optionally wherein the fusion oncoprotein comprises EWS-FLI1, wherein the component comprises 7Ai or variant thereof. In some embodiments, the component targets OTUD7A. In some embodiments, the component blocks and or reduces OTUD7A-mediated deubiquitination of EWS-FLI1 in a cell in the subject. In some embodiments, the component is a catalytic inhibitor, wherein the catalytic inhibitor inhibits the catalytic activity of OTUD7A. In some embodiments, blocking and/or reducing OTUD7A-mediated deubiquitination of EWS-FLI1 substantially destabilizes EWS-FLI1, optionally wherein the stability of EWS-FLI1 is reduced by about 10% to about 90%, or about 20% to about 80%, or about 30% to about 70%, or about 50% or more. In some embodiments, the component substantially reduces EWS-FLI1 levels in the cell. In some embodiments, the 7Ai in such methods comprises the following structure:

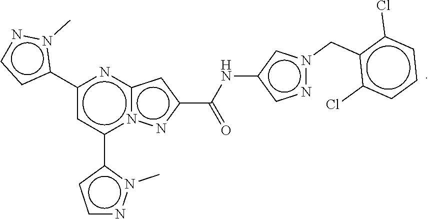

In some embodiments, the subject is suffering from a cancer or is believed to be suffering from a cancer, optionally wherein the cancer is characterized by FLI1 overexpression or dependent on FLI1 for proliferation, optionally wherein the cancer is a leukemia or a kidney cancer. In some embodiments, 7Ai is in the composition at a concentration sufficient to provide a dosage of about 10 μM when administered to a subject, optionally about 1 μM to about 20 M. In some embodiments, the compositions comprise a delivery vehicle for the component, optionally wherein the delivery vehicle is an expression vector, nanoparticle, liposome or vesicle. In some embodiments, the compositions are co-administered to the subject with at least one chemotherapeutic drug.

Also provided herein in some aspects are methods of blocking and/or reducing OTUD7A-mediated deubiquitination of EWS-FLI1 in a cell, the method comprising administering to a cell a composition comprising a component that targets OTUD7A in the cell, wherein the component comprises 7Ai or variant thereof. In some embodiments, the components are a catalytic inhibitor, wherein the catalytic inhibitor inhibits the catalytic activity of OTUD7A. In some embodiments, blocking and/or reducing OTUD7A-mediated deubiquitination of EWS-FLI1 substantially destabilizes EWS-FLI1, optionally wherein the stability of EWS-FLI1 is reduced by about 10% to about 90%, or about 20% to about 80%, or about 30% to about 70%, or about 50% or more. In some embodiments, the 7Ai in the methods of blocking and/or reducing OTUD7A-mediated deubiquitination of EWS-FLI1 in a cell comprises the following structure:

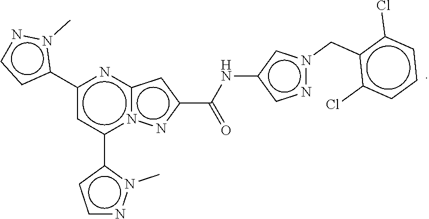

In some embodiments, the cell is in a subject in need of treatment, optionally wherein the subject is suffering from a cancer.

Provided herein are methods of targeting EWS-FLI1 for degradation in a cell, the methods comprising providing 7Ai or a variant thereof to the cell, whereby the 7Ai or variant thereof blocks and/or reduces OTUD7A-mediated deubiquitination of EWS-FLI1 in the cell thereby substantially destabilizing the EWS-FLI1. In some embodiments, the methods are performed in a cell in vivo in a subject. In some embodiments, the subject is a human subject, optionally wherein the subject is suffering from a cancer, optionally wherein the cancer is Ewing sarcoma.

These and other objects are achieved in whole or in part by the presently disclosed subject matter. Other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A through 1T illustrate the results of SPOP targets EWS-FLI1 for ubiquitination and degradation depending on a “VTSSS” (SEQ ID NO: 1) degron in EWS-FLI1. (FIGS. 1A and 1B) Immunoblot (IB) analysis of whole cell lysates (WCL) derived from A673 (A) or SK-N-MC (B) cells treated with 10 μM MG132 for 4 hrs. Cells were lysed in EBC buffer unless specifically noted. Notably, the EWS-FLI1 signal was detected by either an EWSR1-N antibody (A300-417) that can detect both EWSR1 and EWS-FLI1, or an FLI1-C antibody (ab180902) that can detect both FLI1 and EWS-FLI1. (FIG. 1C) IB analysis of WCL derived from A673 cells treated with 1 μM MLN4924 overnight. (FIG. 1D) Sequence alignment of indicated EWS-FLI1 species with canonical SPOP substrates, including amino acids 460-468 of hEWS-FLI1-WT and 408-416 of hFLI1 (i.e., MPVTSSSFF (SEQ ID NO: 95)), amino acids 40-48 of hERG (i.e., MTASSSSDY (SEQ ID NO: 96)), amino acids 605-613 of hDaxx (i.e., GMVSSTSFN (SEQ ID NO: 97)), amino acids 678-687 of hDaxx (i.e., PVADSSTRV (SEQ ID NO: 98)), amino acids 643-651 of hAR (i.e., GEASSTTSP (SEQ ID NO: 99)), amino acids 201-209 of hAR (i.e., VSEGSSSGR (SEQ ID NO: 100)), amino acids 369-377 of hGli (i.e., EQPSSTSGG (SEQ ID NO: 101)), and amino acids 1360-1368 of hGli (i.e., PDVSSSTHP (SEQ ID NO: 102)). (FIG. 1E, FIG. 1F) IB analysis of WCL derived from HEK293T cells transfected with indicated DNA constructs. 100 ng Flag-EWS-FLI1 or Flag-FLI1 construct, together with increasing amounts of GST-SPOP (0, 0.5, 1, 2 μg) or Flag-SPOP (0, 1, 2, 4 μg) were transfected into cells. Notably, same amounts of DNA were transfected in each reaction and the differences in DNA amounts were supplemented with pCDNA3.0. Cells were collected 48 hrs post-transfection unless specified. (FIG. 1G) IB analysis of WCL derived from HEK293T cells transfected with indicated DNA constructs. Where indicated, cells were treated with 10 μM MG132 or 1 μM MLN4924 overnight before cell collection. (FIG. 1H, FIG. 1I) IB analysis of WCL derived from A673 (H) or MHH-ES-1 (I) cells depleted of endogenous SPOP. Cells were infected with lentiviruses targeting indicated targets and selected with 1 μg/mL puromycin for 3 days to eliminate non-infected cells. (FIG. 1J) RT-PCR analyses of EWS-FLI1 mRNA levels in indicated MHH-ES-1 cells. Error bars were calculated as mean+/−SD, n=3. *P<0.05 (one-way ANOVA test). (FIG. 1K) IB analysis of WCL derived from SK-N-MC cells depleted of endogenous CUL3. Cells were infected with lentiviruses targeting cullin 3 and selected with 1 μg/mL puromycin for 3 days to eliminate non-infected cells. (FIG. 1L, FIG. 1M) IB analysis of WCL derived from control or SPOP-depleted MHH-ES-1 cells. Where indicated, 200 μg/mL CHX (cycloheximide) was added to cell culture and cells were harvested at indicated time periods post CHX addition. (M) is a quantification of (L). (FIG. 1N) IB analysis of Ni-NTA pulldowns and WCL derived from HEK293T cells transfected with indicated DNA constructs. Cells were treated with 10 μM MG132 overnight before cell collection. (FIG. 1O) Representative images and quantifications for 3D soft agar assays using indicated cells. Colonies were stained 40 days post-inoculation. Error bars were calculated as mean+/−SD, n=3. *P<0.05 (one-way ANOVA test). The scale bar represents 5 mm. (FIG. 1P) IB analysis of WCL derived from HEK293 cells transfected with 100 ng Flag-EWS-FLI1-WT (which includes at amino acids 460-468 the sequence MPVTSSSFF (SEQ ID NO: 95)) or -3A (which includes at amino acids 460-468 the sequence MPVTAAAFF (SEQ ID NO: 103)) together with 2 μg GST-SPOP constructs. (FIG. 1Q) IB analysis of HA or Flag-IPs and WCL derived from HEK293T cells transfected with indicated DNA constructs. (FIG. 1R) IB analysis of Ni-NTA pulldowns and WCL derived from HEK293T cells transfected with indicated DNA constructs. Cells were treated with 10 μM MG132 overnight before cell collection. (FIG. 1S, FIG. 1T) IB analysis of WCL derived from HEK293T cells transfected with indicated Flag-EWS-FLI1 constructs. Where indicated, 200 μg/mL CHX (cycloheximide) was added to cell culture and cells were harvested at indicated time periods post CHX addition. (T) is a quantification of (S).

FIGS. 2A through 2P are directed to CK1 phosphorylates and primes EWS-FLI1 for SPOP-mediated degradation. (FIG. 2A) IB analysis of WCL derived from HEK293T cells transfected with indicated DNA constructs. Cells were collected 48 hrs post-transfection. (FIG. 2B, FIG. 2C, FIG. 2D) IB analysis of WCL derived from SK-N-MC (B), EWS894 (C) and MHH-ES-1 (D) cells treated with indicated concentrations of CK1 inhibitor D4476 for 16 hrs. (FIG. 2E) IB analysis of WCL derived from MHH-ES-1 cells depleted of endogenous CK1α. Cells were infected with lentiviruses targeting CK1α and selected with 1 μg/mL puromycin for 3 days to eliminate non-infected cells. (FIG. 2F, FIG. 2G) IB analysis of WCL derived from indicated MHH-ES-1 cells treated with 200 μg/mL CHX (cycloheximide) and harvested at indicated time periods. (G) is a quantification of (F). (FIG. 2H) IB analysis of WCL derived from control or endogenous SPOP-depleted A673 cells treated with indicated concentrations of CK1 inhibitor D4476 for 16 hrs. (FIG. 2I) IB analysis of WCL derived from MHH-ES-1 cells treated with indicated concentrations of lenalidomide for 16 hrs. (FIG. 2J) IB analysis of WCL derived from HEK293 cells transfected with 100 ng Flag-EWS-FLI1-WT or -3A together with 2 μg Myc-CK1α constructs. (FIG. 2K) A cartoon illustration of the proposed model: CK1-mediated EWS-FLI1 phosphorylation primes EWS-FLI1 for SPOP recognition and degradation. (FIG. 2L) IB analysis of WCL derived from parental and three isogenic EWS-FLI1-S464A/S465A/S466A knockin A673 cells. (FIG. 2M, FIG. 2N) Representative images for 2D colony formation using cells from (L, #76) and quantified in (N). Error bars were calculated as mean+/−SD, n=3. *P<0.05 (one-way ANOVA test). Scale bar represents 10 mm. (FIG. 2O, FIG. 2P) IB analysis of WCL derived from parental or an isogenic EWS-FLI1-S464A/S465A/S466A knockin A673 (L, #76) cells treated with indicated concentrations of CK1 inhibitor D4476 for 16 hrs.

FIGS. 3A through 3P show genetic inactivation of OTUD7A leads to decreased EWS-FLI1 protein abundance and subsequently impeded Ewing sarcoma cell growth in vitro and in mice. (FIG. 3A) shRNA-mediated OTUB1 and OTUD7A depletion attenuates A673 cell viability. Top panel, illustration of the pipeline for shRNA-guided screen: 3 independent shRNAs targeting each OTU were used to deplete endogenous OTU targets. 3-day post infection, 1,000 cells were plated into 96-well plates in triplicates and cell viability was monitored 3-day post seeding by MTT assays. Error bars were calculated as mean+/−SD, n=3. (FIG. 3B) IB analysis of GST-pulldown and WCL derived from HEK293T cells transfected with indicated DNA constructs. (FIG. 3C) IB analysis of Ni-NTA pulldown and WCL derived from HEK293T cells transfected with indicated DNA constructs. (FIG. 3D, FIG. 3E, FIG. 3F, FIG. 3G) Top panels, IB analysis of WCL derived from A673 (D), MHH-ES-1 (E), EWS894 (F) or MDA-MB-231 (G) cells depleted of endogenous OTUD7A by a tet-on shRNA against endogenous OTUD7A. 1 μg/mL tetracycline was added into cell culture for 72 hrs before cell collection. Bottom panels, representative colony formation assays (D, E, G) or cell growth assays (F) using cells obtained in corresponding top panels. Error bars were calculated as mean+/−SD, n=3 for (D), (F) and n=2 for (E), (G). *P<0.05 (one-way ANOVA test). For (D) and (G) the scale bar represents 5 mm. For (E) the scale bar represents 10 mm. (FIG. 3H) IB analysis of Flag-IPs and WCL derived from HEK293T cells transfected with indicated DNA constructs. (FIG. 3I) IB analysis of WCL derived from parental or EWS-FLI1-3A knockin A673 cells expressing teton-shOTUD7A. Where indicated, 1 μg/mL tetracycline was added into cell culture for 72 hrs before cell collection. (FIG. 3J, FIG. 3K) Representative images for 2D colony formation using cells from (I) and quantified in (K). Error bars were calculated as mean+/−SD, n=2. *P<0.05 (one-way ANOVA test). The scale bar represents 10 mm. (FIG. 3L, FIG. 3M, FIG. 3N) Mouse xenograft experiments were performed with indicated A673 cells. 5-day post-injection when tumors were established in mice, either tetracycline dissolved in water with 1% sucrose, or 1% sucrose dissolved in water only, was fed to mice. Tumor volumes were monitored by caliper measurements at indicated days (L). 25 days post-injection, mice were sacrificed, and tumors were dissected (M) and weighed (N). Error bars were calculated as mean+/−SD, n=9. *P<0.05 (one-way ANOVA test). (FIG. 3O, FIG. 3P) Mouse xenograft experiments were performed with indicated MIH-ES-1 cells. 7-day post-injection when tumors were established in mice, either tetracycline dissolved in water with 1% sucrose, or 1% sucrose dissolved in water only, was fed to mice. 32 days post-injection, mice were sacrificed, and tumors were dissected (O) and weighed (P). Error bars were calculated as mean+/−SD, n=7. *P<0.05 (one-way ANOVA test).

FIGS. 4A through 4O show genetic inactivation of OTUD7A suppresses key EWS-FLI1 downstream signaling. (FIG. 4A) IB analysis of WCL derived from A673 cells treated with dox (tetracycline, 1 μg/mL) for 72 hrs. (FIG. 4B) A cartoon illustration of the working pipeline for TMT labeling and quantitative mass spectrometry analyses. (FIG. 4C) A volcano plot showing down- and up-regulated genes. EWS-FLI1 is indicated in red color. (FIG. 4D) A heatmap summarizing the statistically significantly changed characterized EWS-FLI1 targets in control and OTUD7A depleted A673 cells. (FIG. 4E) IB analyses of WCL derived from WT or 3A-A673 cells infected with Tet-inducible shOTUD7A constructs. Where indicated, cells were collected upon treatment with 1 μg/mL tetracycline (Tet) for indicated periods before cell collection. (FIG. 4F) A growth curve for cells indicated in (G) for indicated time periods measured by cell number. Error bars were calculated as mean+/−SD, n=3. *P<0.05 (one-way ANOVA test). (FIG. 4G, FIG. 4J) RT-PCR analyses of indicated gene changes in control and EWS-FLI1 depleted A673 cells. Lenti-viruses coding shEWS-FLI1 were used to infect A673 cells and selected with 1 μg/mL puromycin to eliminate non-infected cells for 72 hrs before mRNA extraction. Error bars were calculated as mean+/−SD, n=3. *P<0.05 (one-way ANOVA test). (FIG. 4H, FIG. 4I, FIG. 4K, FIG. 4L) RT-PCR analyses of mRNAs derived from indicated cells treated with 1 μg/mL tetracycline (Tet) for indicated periods before cell collection. Error bars were calculated as mean+/−SD, n=3. *P<0.05 (one-way ANOVA test). (FIG. 4M) A heatmap summarizing the statistically significantly changed proteins upon OTUD7A depletion in A673 cells in (A) that are overlapped with a previous proteomic study [45] identifying protein changes upon EWS-FLI1 depletion in A673 cells. Group I: common hits from our study and the previous study [45] that show protein abundance increases upon either EWS-FLI1 or OTUD7A depletion; Group II: cell-cell adhesion proteins showed decreased expression upon OTUD7A depletion but increased expression upon EWS-FLI1 depletion; Group III: proteins showed decreased expression upon EWS-FLI1 depletion but increased expression upon OTUD7A depletion; Group IV: common hits from our study and the previous study [45] that show protein abundance decreases upon either EWS-FLI1 or OTUD7A depletion. (FIG. 4N, FIG. 4O) Representative images for in vitro transwell assays using indicated WT (N) or EWS-FLI1-3A knockin (0) A673-teton-shOTUD7A cells treated with 1 μg/mL tetracycline for 72 hrs before cell fixation and staining. Error bars were calculated as mean+/−SD, n=4. *P<0.05 (one-way ANOVA test). The scale bar represents 50 μm.

FIGS. 5A through 5S show the identification of 7Ai as a lead compound to inhibit OTUD7A activation to suppress Ewing sarcoma growth. (FIG. 5A) Representative IHC images for two Ewing sarcoma tumors obtained from patients stained with indicated antibodies. The scale bar represents 25 μm. C: calvarium; R: rib. (FIG. 5B) IB analysis of WCL derived from A673 cells treated with indicated doses of compound 7Ai for 12 hrs before cell collection. (FIG. 5C) IB analysis of Ni-NTA pulldowns and WCL derived from HEK293T cells transfected with indicated DNA constructs. Where indicated, indicated compounds were added to cell culture 10 hrs prior to cell collection. (FIG. 5D) IB analysis of WCL derived from indicated A673 cells treated with 10 μM compound 7Ai for 12 hrs before cell collection. (FIG. 5E) IB analysis of WCL derived from MHH-ES-1 or EWS894 cells treated with indicated doses of compound 7Ai for 12 hrs before cell collection. (FIG. 5F, FIG. 5G, FIG. 5H) Representative cell viability assays using A673 (F), MHH-ES-1 (G) and EWS894 (H) cells treated with indicated doses of compound 7Ai for 72 hrs before measurements. Error bars were calculated as mean+/−SD, n=3. *P<0.05 (one-way ANOVA test). (FIG. 5I) Representative cell viability assays using A673^{WT} or A673^{3A} cells treated with indicated doses of compound 7Ai for 72 hrs before measurements. Error bars were calculated as mean+/−SD, n=3. *P<0.05 (one-way ANOVA test). (FIG. 5J) Representative images for 2D colony formation by MHH-ES-1 cells treated with indicated doses of compound 7Ai for 14 days. (FIG. 5K, FIG. 5L, FIG. 5M, FIG. 5N) RT-PCR analyses of mRNA level changes of characterized EWS-FLI1 downstream target genes in both WT and EWS-FLI1-3A knocking A673 cells treated with 1 μg/mL tetracycline (Tet) for 3-days including NKX2-2 (K), PSPH (L), LOX (M) and TGFBR2 (N). Error bars were calculated as mean+/−SD, n=3. *P<0.05 (one-way ANOVA test). (FIG. 5O) An illustration of the timeline for 7Ai administration into mice. At indicated periods, 25 mg/kg 7Ai was supplied through IP injection into each mouse. (FIG. 5P, FIG. 5Q, FIG. 5R) Mouse xenograft experiments were performed with A673 cells treated with vehicle or 7Ai. 7-day post-injection when tumors were established in mice, 7Ai (25 mg/kg) was injected through IP route to mice. Tumor volumes were monitored by caliper measurements at indicated days (P). 25 days post-injection, mice were sacrificed and tumors were dissected (Q) and weighed (R). Error bars were calculated as mean+/−SD, n=7. *P<0.05 (one-way ANOVA test). (FIG. 5S) Representative cell viability assays using two Ewing sarcoma cells (A673 and MHH-ES-1) and two normal control cells (HUVEC and FF (foreskin fibroblast)) treated with indicated doses of compound 7Ai for 72 hrs before measurements. Error bars were calculated as mean+/−SD, n=3. *P<0.05 (one-way ANOVA test).

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion.

Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “gene” refers broadly to any segment of DNA associated with a biological function. A gene can comprise sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As is understood in the art, a gene comprises a coding strand and a non-coding strand. As used herein, the terms “coding strand”, “coding sequence” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Also encompassed are any and all nucleotide sequences that encode the disclosed amino acid sequences, including but not limited to those disclosed in the corresponding GENBANK® entries.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell can also affect gene expression as defined herein.

The terms “modulate” or “alter” are used interchangeably and refer to a change in the expression level of a gene, or a level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the terms “modulate” and/or “alter” can mean “inhibit” or “suppress”, but the use of the words “modulate” and/or “alter” are not limited to this definition.

As used herein, the terms “inhibit”, “suppress”, “repress”, “downregulate”, “loss of function”, “block of function”, and grammatical variants thereof are used interchangeably and refer to an activity whereby gene expression (e.g., a level of an RNA encoding one or more gene products) is reduced below that observed in the absence of a composition of the presently disclosed subject matter. In some embodiments, inhibition results in a decrease in the steady state level of a target RNA.

The term “RNA” refers to a molecule comprising at least one ribonucleotide residue. By “ribonucleotide” is meant a nucleotide with a hydroxyl group at the 2′ position of a D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

The term “transcription factor” generally refers to a protein that modulates gene expression, such as by interaction with the cis-regulatory element and/or cellular components for transcription, including RNA Polymerase, Transcription Associated Factors (TAFs), chromatin-remodeling proteins, reverse tet-responsive transcriptional activator, and any other relevant protein that impacts gene transcription.

The term “promoter” defines a region within a gene that is positioned 5′ to a coding region of a same gene and functions to direct transcription of the coding region. The promoter region includes a transcriptional start site and at least one cis-regulatory element. The term “promoter” also includes functional portions of a promoter region, wherein the functional portion is sufficient for gene transcription. To determine nucleotide sequences that are functional, the expression of a reporter gene is assayed when variably placed under the direction of a promoter region fragment.

The terms “active”, “functional” and “physiological”, as used for example in “enzymatically active”, “functional chromatin” and “physiologically accurate”, and variations thereof, refer to the states of genes, regulatory components, chromatin, etc. that are reflective of the dynamic states of each as they exists naturally, or in vivo, in contrast to static or non-active states of each. Measurements, detections or screenings based on the active, functional and/or physiologically relevant states of biological indicators can be useful in elucidating a mechanism, or defining a disease state or phenotype, as it occurs naturally. This is in contrast to measurements taken based on static concentrations or quantities of a biological indicator that are not reflective of level of activity or function thereof.

As used herein, the terms “antibody” and “antibodies” refer to proteins comprising one or more polypeptides substantially encoded by immunoglobulin genes or fragments of immunoglobulin genes. The presently disclosed subject matter also includes functional equivalents of the antibodies of the presently disclosed subject matter. As used herein, the phrase “functional equivalent” as it refers to an antibody refers to a molecule that has binding characteristics that are comparable to those of a given antibody. In some embodiments, chimerized, humanized, and single chain antibodies, as well as fragments thereof, are considered functional equivalents of the corresponding antibodies upon which they are based. In some embodiments, the presently disclosed subject matter provides methods for identifying, characterizing and/or developing disease-related components of a gene-specific chromatin regulatory protein complex, wherein one or more antibodies can be used directly, or in assays related thereto, in the identification, characterization and/or isolation of such components.

The term “substantially identical”, as used herein to describe a degree of similarity between nucleotide sequences, peptide sequences and/or amino acid sequences refers to two or more sequences that have in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99% nucleotide identity, when compared and aligned for maximum correspondence, as measured using a sequence comparison algorithm or by visual inspection.

As used herein, the terms “detectable moiety”, “detectable label”, and “detectable agent” refer to any molecule that can be detected by any moiety that can be added to a chemoprobe, antigen, inhibitor, marker, reagent and/or antibody, or a fragment or derivative thereof, that allows for the detection of the chemoprobe, antigen, inhibitor, marker, reagent and/or antibody, fragment, or derivative in vitro and/or in vivo. Representative detectable moieties include, but are not limited to, chromophores, fluorescent moieties, radioacite labels, affinity probes, enzymes, antigens, groups with specific reactivity, chemiluminescent moieties, and electrochemically detectable moieties, etc. In some embodiments, the antibodies are biotinylated.

The term “prodrug” refers to a derivative of a compound described herein, the pharmacologic action of which results from the conversion by chemical or metabolic processes in vivo to the active compound. Prodrugs can include compounds wherein an amino acid residue, or a polypeptide chain of two or more (e.g., two, three or four) amino acid residues is covalently joined through an amide or ester bond to a free amino, hydroxyl or carboxylic acid group of a compound. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

“Alkyl” refers to a straight or branched hydrocarbon chain radical consisting solely of carbon and hydrogen atoms, containing no unsaturation, having from one to ten carbon atoms (e.g., (C1-10)alkyl or C1-10 alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2 where each Ra is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C2-10)alkenyl or C2-10 alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including IO carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is I or 2), —S(O)tRa (where t is I or 2), —S(O)tORa (where t is I or 2), —S(O)tN(Ra)2 (where t is I or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C2-10)alkynyl or C2-10 alkynyl). Whenever it appears herein, a numerical range such as “2 to IO” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)c(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C3-10)cycloalkyl or C3-10 cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively. The term “alkoxy” refers to the group —O-alkyl, including from I to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O— (substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, —N(Ra)C(NRa)N(Ra)2, —N(Ra)S(O)tRa (where t is I or 2), —S(O)tRa (where t is I or 2), —S(O)tORa (where t is I or 2), —S(O)tN(Ra)2 (where t is I or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)-attached through the carbonyl carbon wherein the alkoxy group has the indicated number of carbon atoms. Thus a (C1-6)alkoxycarbonyl group is an alkoxy group having from I to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is I or 2), —S(O)tRa (where t is I or 2), —S(O)tORa (where t is I or 2), —S(O)tN(Ra)2 (where t is I or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is I or 2), —S(O)tRa (where t is I or 2), —S(O)tORa (where t is I or 2), —S(O)tN(Ra)2 (where t is I or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is I or 2), —S(O)tRa (where t is I or 2), —S(O)tORa (where t is I or 2), —S(O)tN(Ra)2 (where t is I or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acylsulfonamide” refers a —S(O)2-N(Ra)-C(═O)— radical, where Ra is hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Unless stated otherwise specifically in the specification, an acylsulfonamide group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is I or 2), —S(O)tRa (where t is I or 2), —S(O)tORa (where t is I or 2), —S(O)tN(Ra)2 (where t is I or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(Ra)2 radical group, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(Ra)2 group has two Ra substituents other than hydrogen, they can be combined with the nitrogen atom to form a 4-, 5-, 6- or 7-membered ring. For example, —N(Ra)2 is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)c(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHRa, and —NRaRa each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)2 or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R2 of —N(R)2 of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3rd Ed., John Wiley & Sons, New York, N.Y., 1999, which is incorporated herein by reference in its entirety.

“Aromatic” or “aryl” or “Ar” refers to an aromatic radical with six to ten ring atoms (e.g., C6-C10 aromatic or C6-C10 aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)c(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PO3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “aryloxy” refers to the group —O-aryl.

The term “substituted aryloxy” refers to aryloxy wherein the aryl substituent is substituted (i.e., —O-(substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)c(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PO3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3rd Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)c(NRa)N(Ra)2, —N(Ra)S(O)tRa (where t is I or 2), —S(O)tRa (where t is I or 2), —S(O)tORa (where t is I or 2), —S(O)tN(Ra)2 (where t is I or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C1-C4 heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —ORa, —SRa, —OC(O)—Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)c(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively. [0075] “Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively. [0077] “Heteroaryl” or “heteroaromatic” or “HetAr” or “Het” refers to a 5- to 18-membered aromatic radical (e.g., Cs—Cn heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. AN-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9, I 0-hexahydrocycloocta[d]pyrimidinyl, 5,6,7,8,9, I 0-hexahydrocycloocta[d]pyridazinyl, 5,6,7,8,9, I 0-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9, I 0, I 0a-octahydrobenzo[h]quinazolinyl, 1-phenyl-IH-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —ORa, —SRa, —OC(O)Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is I or 2), —S(O)tRa (where t is I or 2), —S(O)tORa (where t is I or 2), —S(O)tN(Ra)2 (where t is I or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —ORa, —SRa, —OC(O)— Ra, —SC(O)—Ra, —N(Ra)2, —C(O)Ra, —C(O)ORa, —C(O)SRa, —OC(O)N(Ra)2, —C(O)N(Ra)2, —N(Ra)C(O)ORa, —N(Ra)C(O)Ra, —N(Ra)C(O)N(Ra)2, N(Ra)C(NRa)N(Ra)2, —N(Ra)S(O)tRa (where t is 1 or 2), —S(O)tRa (where t is 1 or 2), —S(O)tORa (where t is 1 or 2), —S(O)tN(Ra)2 (where t is 1 or 2), or PQ3(Ra)2, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO2 radical.

“Oxa” refers to the -0- radical.

“Oxo” refers to the ═O radical.

“Isomers” are different compounds that have the same molecular formula.

“Stereoisomers” are isomers that differ only in the way the atoms are arranged in space—i.e., having a different stereochemical configuration.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate.

“Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line.

Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O2)-H, —S(O2)-(optionally substituted alkyl), —S(O2)-(optionally substituted amino), —S(O2)-(optionally substituted aryl), —S(O2)-(optionally substituted heteroaryl), and —S(O2)-(optionally substituted heterocycloalkyl). [0098] “Sulfonamidyl” or “sulfonamido” refers to a —S(═O)2-NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)2-NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)2OH radical.

“Sulfonate” refers to a —S(═O)2-OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively. [00101] Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### Pharmaceutical Compositions

The disclosure provides a pharmaceutical composition for use in the treatment of the diseases and conditions described herein. In some embodiments, the disclosure provides a pharmaceutical composition comprising a therapeutically effective amount of a component sufficient to block and/or reduce OTUD7A-mediated deubiquitination of EWS-FLI1 in a cell, wherein the component comprises 7Ai and variants thereof.

The pharmaceutical compositions are typically formulated to provide a therapeutically effective amount of 7Ai or variant thereof. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

In some embodiments, the concentration of 7Ai or variant thereof provided in the pharmaceutical compositions of the disclosure is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%1, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of 7Ai or variant thereof provided in the pharmaceutical compositions of the disclosure is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of 7Ai or variant thereof provided in the pharmaceutical compositions of the disclosure is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of 7Ai or variant thereof provided in the pharmaceutical compositions of the disclosure is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of 7Ai or variant thereof provided in the pharmaceutical compositions of the disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of any one of the pharmaceutical compositions of the disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, Ig, 1.5 g, 2 g, 2.5 g, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the compounds provided according to the disclosure is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Oral Administration

In preferred embodiments, the disclosure provides a pharmaceutical composition for oral administration containing 7Ai or variant thereof and their features and limitations as described herein, or pharmaceutically acceptable analogs, derivatives, salts, solvates, hydrates, cocrystals, or prodrugs thereof, described herein, and a pharmaceutical excipient suitable for administration.

In preferred embodiments, the disclosure provides a solid pharmaceutical composition for oral administration containing 7Ai or variant thereof and their features and limitations as described herein, or pharmaceutically acceptable analogs, derivatives, salts, solvates, hydrates, cocrystals, or prodrugs thereof, and a pharmaceutical excipient suitable for administration. In some embodiments, the composition further contains an effective amount of an additional active pharmaceutical ingredient. Such additional active pharmaceutical ingredients may also include those compounds used for sensitizing cells to additional agent(s).

In some embodiments, the pharmaceutical composition may be a liquid pharmaceutical composition suitable for oral consumption.

Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may beaded (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about I to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, asyloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof, carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-JO laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-JO oleyl ether, POE-20 oleyl ether, POE-20 stearyl ether, tocopheryl PEG-I 00 succinate, PEG-24 cholesterol, polyglyceryl-10 oleate, Tween 40, Tween 60, sucrose monostearate, sucrose monolaurate, sucrose monopalmitate, PEG I 0-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycolalkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides. [00163] In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, E-caprolactam, N-alkylpyrrolidone, N-hydroxyalky lpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, 8-valerolactone and isomers thereof, -butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethyl pyrrolidone, polyvinylpyrrolidone, hydroxypropyl methyl cellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of I 0%, 25%, 50%, I 00%, or up to about 200% by weight, based on the combined weight of the drug, and other excipients. If desired, very small amounts of solubilizer may also be used, such as 5%, 2%, I % or even less. Typically, the solubilizer may be present in an amount of about I % to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the disclosure provides a pharmaceutical composition for injection containing 7Ai or variant thereof and a pharmaceutical excipient suitable for injection. Components and amounts of compounds in the compositions areas described herein.

The forms in which the compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating 7Ai or variant thereof in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In preferred embodiments, the disclosure provides a pharmaceutical composition for transdermal delivery containing 7Ai or variant thereof and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of 7Ai or variant thereof in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

The disclosure also provides kits. The kits include 7Ai or variant thereof in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient.

Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The kits described above are preferably for use in the treatment of the diseases and conditions described herein. In a preferred embodiment, the kits are for use in the treatment of cancer or hyperproliferative disorders. In an embodiment, the kits described herein are for use in the treatment of EWS.

### Dosages and Dosing Regimens

The amounts of 7Ai or variant thereof administered, will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage may be provided in units of mg/kg of body mass or in mg/m² of body surface area.

In some embodiments, dosing may be once, twice, three times, four times, five times, six times, or more than six times per day. Dosing may be once a month, once every two weeks, once a week, or once every other day. In some embodiments, 7Ai or variant thereof is administered for more than 1, 2, 3, 4, 5, 6, 7, 14, or 28 days. In some embodiments, 7Ai or variant thereof is administered for less than 28, 14, 7, 6, 5, 4, 3, 2, or 1 day. In yet another embodiment, the administration continues for more than about 6, 10, 14, 28 days, two months, six months, or one year. In some cases, continuous dosing is achieved and maintained as long as necessary.

In some embodiments, an effective dosage is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg.

In some embodiments, an effective dosage is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### II. Subjects

The subject treated, screened, tested, or to which a dosage is administered, is desirably a human subject, although it is to be understood that the principles of the disclosed subject matter indicate that the compositions and methods are effective with respect to invertebrate and to all vertebrate species, including mammals, which are intended to be included in the term “subject”. Moreover, a mammal is understood to include any mammalian species in which screening is desirable, particularly agricultural and domestic mammalian species.

The disclosed methods and treatments are particularly useful in the treatment of warm-blooded vertebrates. Thus, the presently disclosed subject matter concerns mammals and birds.

### III. General Discussion

Ewing sarcoma is an aggressive malignancy that develops in bones or soft tissues of children and young adults. A recurrent chromosomal translocation found in the majority of Ewing sarcoma fuses the EWSR1 and FLI1 genes generating an EWS-FLI1 fusion protein. EWS-FLI1 is the critical driver of Ewing sarcoma. Mechanistically, EWS-FLI1 binds specific GGAA-containing microsatellite regions to maintain nucleosome depletion. EWS-FLI1 recruits a set of chromatin and transcriptional regulators, including BRG1, RNA polymerase II, CBP/p300, RNA helicase A and others, to modulate transcription of target genes, including NR0B1, GLI1, FOXOs, LOX, IGF1 and others that maintain properties of malignant transformation. However, recent studies indicate that EWS-FLI1 does not act in a binary fashion; rather EWS-FLI1 expression levels influence cellular states. High levels of EWS-FLI1 are associated with an immature, proliferative phenotype, whereas reduced levels correlate with decreased proliferation and a more motile cellular phenotype.

As the EWS-FLI1 fusion occurs exclusively in the tumor cells, it is considered as an ideal target to treat Ewing sarcoma. Prior efforts to identify and target major EWS-FLI1 downstream genes have not been effective. Further, direct targeting of EWS-FLI1 has been hampered by the lack of enzymatic activity and suitable small molecule interaction domains. Notably, a small molecule enantiomer-specific EWS-FLI1 inhibitor TK-216 was identified and being tested in early clinical development. Recent efforts aim to block EWS-FLI1 interaction with DNA [14] or modulate its ability to affect chromatin states. Targeting EWS-FLI1 protein stability constitutes a potential therapeutic strategy. Although proteasome-mediated and lysosome-controlled EWS-FLI1 degradation have been reported, the identities of E3 ligase(s) and deubiquitinase(s) responsible for EWS-FLI1 protein stability control remain elusive. USP7 was identified from a CRISPR screen as a dependency for p53-WT Ewing sarcoma and the deubiquitinase USP19 was found to stabilize both EWS-FLI1 and EWSR1 proteins. However, the multiple roles of USP7 on targeting both tumor suppressors and oncogenes, as well as the pleiotropy of USP19 complicate their applications to treat Ewing sarcoma. Although inhibitors of USP7 and USP19 have been developed, while their effects on Ewing sarcoma remain to be determined.

Chromosomal translocation results in the development of the EWS-FLI1 fusion oncogene in the majority of Ewing sarcoma. The persistent dependence of the tumor for this oncoprotein points to EWS-FLI1 as an ideal drug target. Although EWS-FLI1 transcriptional targets and binding partners have been evaluated, the mechanisms regulating EWS-FLI1 protein stability have remained elusive. As disclosed herein for the first time, SPOP and OTUD7A were identified as bonafide E3 ligase and deubiquitinase, respectively, that control EWS-FLI1 protein turnover in Ewing sarcoma. CK1-mediated phosphorylation of the VTSSS (SEQ ID NO: 1) degron in the FLI1 domain enhances SPOP activity to degrade EWS-FLI1. Opposing this process, OTUD7A deubiquitinates and stabilizes EWS-FLI1. Depletion of OTUD7A in Ewing sarcoma cell lines reduced EWS-FLI1 protein abundance and impeded Ewing sarcoma growth in vitro and in mice. Performing an AI-based virtual drug screen of a 4-million small molecule library, 7Ai was identified as a potential OTUD7A catalytic inhibitor.

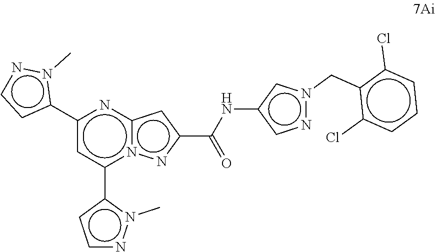

7Ai reduced EWS-FLI1 protein levels and decreased Ewing sarcoma growth in vitro and in vivo in a xenograft mouse model. The instant disclosure supports the therapeutic in vivo targeting of OTUD7A as a novel strategy for Ewing sarcoma bearing EWS-FLI1 and related fusions, and is also applicable to other cancers dependent on aberrant FLI1 expression.

Thus, in some embodiments, provided herein are compositions for targeting OTUD7A, the compositions comprising a component sufficient to block and/or reduce OTUD7A-mediated deubiquitination of EWS-FLI1 in a cell (in vivo or in vitro). The component can be a catalytic inhibitor, wherein the catalytic inhibitor inhibits the catalytic activity of OTUD7A. In some aspects, blocking and/or reducing OTUD7A-mediated deubiquitination of EWS-FLI1 substantially destabilizes EWS-FLI1. In some embodiments, the stability of EWS-FLI1 is reduced by about 10% to about 90%, or about 20% to about 80%, or about 30% to about 70%, or about 50% or more. The component can substantially reduce EWS-FLI1 levels in the cell. The cell can be a human cell. In some aspects, the composition can be configured for in vivo administration to a subject.

In some embodiments, the component of such compositions can comprise 7Ai or a variant thereof. The 7Ai can be in the composition at a concentration sufficient to provide a dosage of about 10 μM when administered to a subject, optionally about 1 μM to about 20 M. Such compositions can comprise a delivery vehicle for the component, optionally wherein the delivery vehicle is an expression vector, nanoparticle, liposome or vesicle. The compositions can be configured for treating a cancer, optionally wherein the cancer is Ewing sarcoma.

Also provided herein are OTUD7A catalytic inhibitors, the catalytic inhibitors comprising a 7Ai compound or variant thereof. The catalytic inhibitors can be configured to substantially limit Ewing sarcoma growth in vivo by degrading EWS-FLI1.

In some aspects, provided herein are methods of treating Ewing sarcoma and related conditions, the methods comprising administering to a subject having Ewing sarcoma or suspected of suffering from Ewing sarcoma a composition comprising a component targeting a fusion oncoprotein, optionally wherein the fusion oncoprotein comprises EWS-FLI1, wherein the component is 7Ai or variant thereof. In some embodiments, the component targets OTUD7A. In some embodiments, the component blocks and/or reduces OTUD7A-mediated deubiquitination of EWS-FLI1 in a cell in the subject. The component can be a catalytic inhibitor, wherein the catalytic inhibitor inhibits the catalytic activity of OTUD7A, wherein the catalytic inhibitor is 7Ai or variant thereof. In some aspects, blocking and/or reducing OTUD7A-mediated deubiquitination of EWS-FLI1 substantially destabilizes EWS-FLI1, optionally wherein the stability of EWS-FLI1 is reduced by about 10% to about 90%, or about 20% to about 80%, or about 30% to about 70%, or about 50% or more. The component can substantially reduce EWS-FLI1 levels in the cell. The component can comprise 7Ai or a variant thereof. In some aspects, the subject can be suffering from a cancer or is believed to be suffering from a cancer, optionally wherein the cancer is characterized by FLI1 overexpression or dependent on FLI1 for proliferation, optionally wherein the cancer is leukemia or kidney cancer.

Also provided herein are methods of blocking and/or reducing OTUD7A-mediated deubiquitination of EWS-FLI1 in a cell, the methods comprising administering to a cell a composition comprising a component that targets OTUD7A in the cell, wherein the component is 7Ai or variant thereof. The component can be a catalytic inhibitor, wherein the catalytic inhibitor inhibits the catalytic activity of OTUD7A, wherein the component is 7Ai or variant thereof. Blocking and/or reducing OTUD7A-mediated deubiquitination of EWS-FLI1 substantially destabilizes EWS-FLI1, optionally wherein the stability of EWS-FLI1 is reduced by about 10% to about 90%, or about 20% to about 80%, or about 30% to about 70%, or about 50% or more. In some aspects, the component can comprise 7Ai or a variant thereof. The cell can be in a subject in need of treatment, optionally wherein the subject is suffering from a cancer.

In some embodiments, provided herein are methods of targeting EWS-FLI1 for degradation in a cell, the methods comprising providing 7Ai or a variant thereof to the cell, whereby the 7Ai or variant thereof blocks and/or reduces OTUD7A-mediated deubiquitination of EWS-FLI1 in the cell thereby substantially destabilizing the EWS-FLI1. Such methods can be performed in a cell in vivo in a subject. In some aspects, the subject can be a human subject, optionally wherein the subject is suffering from a cancer, optionally wherein the cancer is Ewing sarcoma.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-11

### Cell Culture and Transfection

HEK293, HEK293T, FF (foreskin fibroblast) and ACHN cells were cultured in DMEM medium supplemented with 10% FBS. A673, MIH-ES-1 and MDA-MB-231 cells were cultured in DMEM medium supplemented with 10% FBS. Jurkat and CUTLL1 cells were cultured in RPMI-1640 medium supplemented with 10% FBS. EWS502 and EWS894 cells were maintained in RPMI-1640 medium supplemented with 15% FBS. SK-N-MC were cultured in RPMI-1640 medium supplemented with 10% FBS, 200 μM glutamine (Gibco, 25030081) and non-essential amino acids (Gibco, 11140050). HUVEC cells were cultured in Endothelial Cell Growth Medium 2 (PromoCell, C-22111) supplemented with 10% FBS. All cell culture media were supplemented with 100 units of penicillin and 100 mg/mL streptomycin unless otherwise stated.

Cell transfection was performed using lipofectamine 3000 or polyethylenimine (PEI), as described previously [52-54]. Packaging of lentiviral shRNA or cDNA expressing viruses, as well as subsequent infection of various cell lines were performed according to the protocols described previously [55-56]. Following viral infection, cells were maintained in the presence of blasticidin (5 μg/mL) or puromycin (1 μg/mL), depending on the viral vector used to infect cells.

MG132 (S2619), MLN4924 (S7109), cycloheximide (S6611), D4476 (S7642) and lenalidomide (S1029) were purchased from Selleck. Tetracycline (87128) and doxycycline (D9891) were purchased from Sigma-Aldrich. JQ1 was purchased from Sigma (SML0974). Larger quantities of compound 7Ai was purchased from princetonbio.

### Plasmids Construction

Flag-SPOP and CMV-GST-SPOP are as described in [29]. pCDNA3-HA-SPOP plasmid was constructed by cloning SPOP into pCDNA3-HA vector using primers listed below. Myc tagged CK1s and CK2s are as described [29]. His-ub plasmids are as described [57]. Myc-Flag-OTU plasmids are as described [40]. His-OTUD7A was constructed by cloning OTUD7A into pET28a vector using primers listed below. HA-EWS-FLI1 and HA-FLI1 were cloned into pCDNA3-HA vector using primers listed below. pLenti-HA-FLI1-WT and 3A plasmids were cloned into the pLenti-HA-hygro vector using primers listed below. HA-EWSR1, and pLL5.5-HA-EWS-ERG were generated in the Davis lab. HA-SPOP was cloned into pCDNA3-HA vector using primers listed below. His-OTUD7A was cloned into pET28a vector using primers listed below. pLenti-EWS-FLI1-3A was constructed by cloning EWS-FLI1-3A into pLenti-GFP-hygro vector using primer below from Flag-EWS-FLI1-3A plasmid. Myc-Cullin plasmids were a generous gift from Yue Xiong lab at University of North Carolina at Chapel Hill.

EWS-FLI-BglII-F:
(SEQ ID NO: 2)
GCATAGATCTGCGTCCACGGATTACAGTACC
FLI1-BglII-F:
(SEQ ID NO: 3)
GCATAGATCTGACGGGACTATTAAGGAGGC
FLI1-XhoI-R:
(SEQ ID NO: 4)
GCATCTCGAGCTAGTAGTAGCTGCCTAAGTG
hSPOP-BamHI-F:
(SEQ ID NO: 5)
GCATGGATCCTCAAGGGTTCCAAGTCCTCCAC
hSPOP-XhoI-R:
(SEQ ID NO: 6)
GCATCTCGAGTTAGGATTGCTTCAGGCGTTTGCG
OTUD7A-BglII-F:
(SEQ ID NO: 7)
GCATAGATCTGTTTCTAGTGTGCTTCCAAACC
OTUD7A-SalI-R:
(SEQ ID NO: 8)
GCATGTCGACTCACAGCTCCTCGCGG

EWS-FLI1-3A, OTUD7A-C210S and CUL3-E358Q mutants were generated using the QuikChange XL Site-Directed Mutagenesis Kit (Stratagene) according to the manufacturer's instructions. Details of plasmid constructions are available upon request.

EWS-FLI-3A-F:
(SEQ ID NO: 9)
CCTCCATGCCTGTCACTGCCGCCGCCTTCTTTGGAGCCGCATCAC
EWS-FLI-3A-R:
(SEQ ID NO: 10)
GTGATGCGGCTCCAAAGAAGGCGGCGGCAGTGACAGGCATGGAGG
OTUD7A-C210S-F:
(SEQ ID NO: 11)
CAGGGGATGGGAACTCCCTTTTACATGCTGCTTCACTG
OTUD7A-C210S-R:
(SEQ ID NO: 12)
CAGTGAAGCAGCATGTAAAAGGGAGTTCCCATCCCCTG
CUL3-E358Q-F:
(SEQ ID NO: 13)
GTTCGATCGCTTCCTCCTGCAATCATTCAACAATGACCGTCTC
CUL3-E358Q-R:
(SEQ ID NO: 14)
GAGACGGTCATTGTTGAATGATTGCAGGAGGAAGCGATCGAAC

RT-PCR primers to examine EWS-FLI1 mRNA changes upon SPOP or OTUD7A depletion are listed below.

EWS-F:
(SEQ ID NO: 15)
TCCTACAGCCAAGCTCCAAGTC
FLI1-R:
(SEQ ID NO: 16)
ACTCCCCGTTGGTCCCCTCC

RT-PCR primers to examine EWS-FLI1 transcriptional targets used in this study are listed below:

EWS-FLI1-F:
(SEQ ID NO: 17)
CAGTCACTGCACCTCCATCC
EWS-FLI1-R:
(SEQ ID NO: 18)
TTCATGTTATTGCCCCAAGC
NKX2-2-F:
(SEQ ID NO: 19)
CTACGACAGCAGCGACAACC
NKX2-2-R:
(SEQ ID NO: 20)
GCCTTGGAGAAAAGCACTCG
TGFBR2-F:
(SEQ ID NO: 21)
CATCTGTGAGAAGCCACAGG
TGFBR2-R:
(SEQ ID NO: 22)
TGCACTCATCAGAGCTACAGG
IGFBP3-F:
(SEQ ID NO: 23)
CTGCTCAGATTTCCCCAAAG
IGFBP3-R:
(SEQ ID NO: 24)
TGGCATCAAGCAGGTCATAG
LOX-F:
(SEQ ID NO: 25)
CATCAAGAAAGGGCATGCTAA
LOX-R:
(SEQ ID NO: 26)
CTACGGCAGGGACCATATTCT
JAK1-F:
(SEQ ID NO: 27)
CAGGTCTCCCACAAACACATCG
JAK1-R:
(SEQ ID NO: 28)
ACCAGGTCTTTATCCTCCAAGTAGC
CCND1-F:
(SEQ ID NO: 29)
CGCACGATTTCATTGAACACTT
CCND1-R:
(SEQ ID NO: 30)
CGGATTGGAAATACTTCACAT
CCND3-F:
(SEQ ID NO: 31)
CCTCTGTGCTACAGATTATACCTTTGC
CCND3-R:
(SEQ ID NO: 32)
TTGCACTGCAGCCCCAAT
GSTM4-F:
(SEQ ID NO: 33)
TGGAGAACCAGGCTATGGACGT
GSTM4-R:
(SEQ ID NO: 34)
CCAGGAACTGTGAGAAGTGCTG
PHGDH-F:
(SEQ ID NO: 35)
CTGCGGAAAGTGCTCATCAGT
PHGDH-R:
(SEQ ID NO: 36)
TGGCAGAGCGAACAATAAGGC
PSPH-F:
(SEQ ID NO: 37)
GATGCTGTGTGTTTTGATGTTGAC
PSPH-R:
(SEQ ID NO: 38)
CTTGACTTGTTGCCTGATCACATT
SLC1A5-F:
(SEQ ID NO: 39)
CTTGGTAGTGTTTGCCATCGT
SLC1A5-R:
(SEQ ID NO: 40)
TGCGGGTGAAGAGGAAGTAG
MTHFD1L-F:
(SEQ ID NO: 41)
GAGCTCTGAAGARGCATGGAG
MTHFD1L-R:
(SEQ ID NO: 42)
TGCTTCTGGAGGTTACAGCA

shRNAs and sgRNAs

shRNA vectors to deplete endogenous SPOP, CUL3 and various OTUs were purchased from Sigma. Their sequence was listed below:

shSPOP-1:
(SEQ ID NO: 43)
CCGGCACAGATCAAGGTAGTGAAATCTCGAGATTTCACTACCTTG
ATCTGTGTTTTTTG
shSPOP-2:
(SEQ ID NO: 44)
CCGGCAAGGTAGTGAAATTCTCCTACTCGAGTAGGAGAATTTCAC
TACCTTGTTTTTTG
shSPOP-3:
(SEQ ID NO: 45)
CCGGCAAACGCCTGAAGCAATCCTACTCGAGTAGGATTGCTTCAG
GCGTTTGTTTTTTG
shSPOP-4:
(SEQ ID NO: 46)
CCGGCTCCTACATGTGGACCATCAACTCGAGTTGATGGTCCACAT
GTAGGAGTTTTTTG
shCUL3-1:
(SEQ ID NO: 47)
CCGGCGTGTGCCAAATGGTTTGAAACTCGAGTTTCAAACCATTTG
GCACACGTTTTTG
shCUL3-2:
(SEQ ID NO: 48)
CCGGTTCAGGCTTTACAACGTTTATCTCGAGATAAACGTTGTAAA
GCCTGAATTTTTG
shCUL3-3:
(SEQ ID NO: 49)
CCGGCGTGTGCCAAATGGTTTGAAACTCGAGTTTCAAACCATTTG
GCACACGTTTTTG
shOTUB1-1:
(SEQ ID NO: 50)
CCGGAGGAGTATGCTGAAGATGACACTCGAGTGTCATCTTCAGCA
TACTCCTTTTTT
shOTUB1-2:
(SEQ ID NO: 51)
CCGGTGTTTCTATCGGGCTTTCGGACTCGAGTCCGAAAGCCCGAT
AGAAACATTTTT
shOTUB1-3:
(SEQ ID NO: 52)
CCGGTGTGGTTGTAAATGGTCCTATCTCGAGATAGGACCATTTAC
AACCACATTTTT
shOTUB2-1:
(SEQ ID NO: 53)
CCGGCCTATGTGTCACTGGATTATTCTCGAGAATAATCCAGTGAC
ACATAGGTTTTTG
shOTUB2-2:
(SEQ ID NO: 54)
CCGGTGGGCTGCTATGTCTCTGTATCTCGAGATACAGAGACATAG
CAGCCCATTTTT
shOTUB2-2:
(SEQ ID NO: 55)
CCGGCCTTCCGTTTACCTGCTCTATCTCGAGATAGAGCAGGTAAA
CGGAAGGTTTTT
shOTUD3-1:
(SEQ ID NO: 56)
CCGGGACGTCTGCCATCGCATATTACTCGAGTAATATGCGATGGC
AGACGTCTTTTTG
shOTUD3-2:
(SEQ ID NO: 57)
CCGGTTTGGAAATCAGGGCTTAAATCTCGAGATTTAAGCCCTGAT
TTCCAAATTTTTG
shOTUD3-3:
(SEQ ID NO: 58)
CCGGGGGAGTTACACATCGCATATCCTCGAGGATATGCGATGTGT
AACTCCCTTTTTG
shOTUD4-1:
(SEQ ID NO: 59)
CCGGCAAGTCGAGAATCTAACTATTCTCGAGAATAGTTAGATTCT
CGACTTGTTTTTG
shOTUD4-2:
(SEQ ID NO: 60)
CCGGTATGCAATGCCTTAGTCATAACTCGAGTTATGACTAAGGCA
TTGCATATTTTTG
shOTUD4-3:
(SEQ ID NO: 61)
CCGGCACTATAGATTCCAAACATAACTCGAGTTATGTTTGGAATC
TATAGTGTTTTTG
shOTUD5-1:
(SEQ ID NO: 62)
CCGGCCATCATTCAAACCAGGGTTTCTCGAGAAACCCTGGTTTGA
ATGATGGTTTTTTG
shOTUD5-2:
(SEQ ID NO: 63)
CCGGCCGACTACTTCTCCAACTATGCTCGAGCATAGTTGGAGAAG
TAGTCGGTTTTTG
shOTUD5-3:
(SEQ ID NO: 64)
CCGGAGAACGTCTGAGCCTTCAATGCTCGAGCATTGAAGGCTCAG
ACGTTCTTTTTTG
shOTUD6A-1:
(SEQ ID NO: 65)
CCGGCATGATCTACTGCGACAACATCTCGAGATGTTGTCGCAGTA
GATCATGTTTTTTG
shOTUD6A-2:
(SEQ ID NO: 66)
CCGGCACCAACTAAGATTTGGTCATCTCGAGATGACCAAATCTTA
GTTGGTGTTTTTTG
shOTUD6A-3:
(SEQ ID NO: 67)
CCGGGATTTGGTCATGTTGCGTATACTCGAGTATACGCAACATGA
CCAAATCTTTTTTG
shOTUD6B-1:
(SEQ ID NO: 68)
CCGGGCAAAGCTACTAACAGGTGTTCTCGAGAACACCTGTTAGTA
GCTTTGCTTTTTTG
shOTUD6B-2:
(SEQ ID NO: 69)
CCGGGCTGACTACTAAGGAGAATAACTCGAGTTATTCTCCTTAGT
AGTCAGCTTTTTTG
shOTUD6B-3:
(SEQ ID NO: 70)
CCGGCGATGAGACTAATGCAGTGAACTCGAGTTCACTGCATTAGT
CTCATCGTTTTTTG
shOTUD7A-1:
(SEQ ID NO: 71)
CGGGCAGCAATTCTAACAGCAATACTCGAGTATTGCTGTTAGAAT
TGCTGCTTTTTG
shOTUD7A-2:
(SEQ ID NO: 72)
CCGGCGCACACACTTCAGCAAGAATCTCGAGATTCTTGCTGAAGT
GTGTGCGTTTTTG
shOTUD7A-3:
(SEQ ID NO: 73)
CCGGGCGCGAGAACTGTGCGTTCTACTCGAGTAGAACGCACAGTT
CTCGCGCTTTTTG
shOTUD7B-1:
(SEQ ID NO: 74)
GTACCGGTTGAAGAGTTTCACGTCTTTGCTCGAGCAAAGACGTGA
AACTCTTCAATTTTTTG
shOTUD7B-2:
(SEQ ID NO: 75)
CCGGTGGAAATGCTCACGGTTTATACTCGAGTATAAACCGTGAGC
ATTTCCATTTTTG
shOTUD7B-3:
(SEQ ID NO: 76)
CCGGGCAAGGAGGCTAAACAAAGTTCTCGAGAACTTTGTTTAGCC
TCCTTGCTTTTT
shCK1α-42:
(SEQ ID NO: 77)
CCGGGCAGAATTTGCGATGTACTTACTCGAGTAAGTACATCGCAA
ATTCTGCTTTTT
shCK1α-87:
(SEQ ID NO: 78)
CCGGGCAAGCTCTATAAGATTCTTCCTCGAGGAAGAATCTTATAG
AGCTTGCTTTTTTG
shFLI1:
(SEQ ID NO: 79)
TGCCCATCCTGCACACTTACTTCAAGAGAGTAAGTGTGCAGGATG
GGCTTTTTTC
(targeting the 3'UTR region of
FLI1 as reported in [2])

Teton-shOTUD7 primers are listed below:

Teton-shOTUD7A-F:
(SEQ ID NO: 80)
CCGGGCGCGAGAACTGTGCGTTCTACTCGAGTAGAACGCACAGTT
CTCGCGCTTTTT
Teton-shOTUD7A-R:
(SEQ ID NO: 81)
AATTAAAAAGCGCGAGAACTGTGCGTTCTACTCGAGTAGAACGCA
CAGTTCTCGCGC

shOTUD7A-resistant OTUD7A construct was generated using the QuikChange XL Site-Directed Mutagenesis Kit (Stratagene) according to the manufacturer's instructions.

shOTUD7A-62-resistant-F:
(SEQ ID NO: 82)
CTGCCAGCGGGAAAATTGCGCGTTCTACGG
shOTUD7A-62-resistant-R:
(SEQ ID NO: 83)
CCGTAGAACGCGCAATTTTCCCGCTGGCAG

EWS-FLI1-3A knockin experiment was performed using EWS-FLI1 sgRNAs and ssoDNA as listed below:

EWS-FLI1-3A-sgRNA-F:
(SEQ ID NO: 84)
CACCG TGCGGCTCCAAAGAAGCTGG
EWS-FLI1-3A-sgRNA-R:
(SEQ ID NO: 85)
AAAC CCAGCTTCTTTGGAGCCGCA C
EWS-FLI1-3A-ssoDNA:
(SEQ ID NO: 86)
GCCCACCAGCAGAAGGTGAACTTTGTCCCTCCCCATCCATCCTCC
ATGCCTGTCACTGCCGCCGCCTTCTTTGGAGCCGCATCACAATAC
TGGACCTCCCCCACGGGGGGAATCTACCCC

Knockin clones were screened by PCR using primers listed below to search for clones loss of BpmI site after knockin.

EWSFLI-3A-KI-PCR-F:
(SEQ ID NO: 87)
GTGCACGGCAAAAGATATGCTTAC
EWSFLI-3A-KI-PCR-R:
(SEQ ID NO: 88)
CTAGTAGTAGCTGCCTAAGTGTG

sgRNAs to stably deplete endogenous OTUD7A are listed below:

sgOTUD7A-1A-F:
(SEQ ID NO: 89)
CACCGAGACTTGTTCGGTCCACGG
sgOTUD7A-1A-R:
(SEQ ID NO: 90)
AAACCCGTCCACCGAACAAAGTCTC
sgOTUD7A-1B-F:
(SEQ ID NO: 91)
CACCGTGCTGCCCAACACTCAGCCG
sgOTUD7A-1B-R:
(SEQ ID NO: 92)
AAACCGGCTGAGTGTTGGGAGCAC
sgOTUD7A-1C-F:
(SEQ ID NO: 93)
CACGCAGACCAGGTTCTGCCCCCG
sgOTUD7A-1C-R:
(SEQ ID NO: 94)
AAACCGGGGGCAGAACCTGGTCTGC

### Immunoblot and Immunoprecipitations Analyses

Cells were lysed in EBC buffer (50 mM Tris pH 7.5, 120 mM NaCl, 0.5% NP-40) or Triton X-100 buffer (50 mM Tris pH 7.5, 150 mM NaCl, 1% Triton X-100) supplemented with protease inhibitors (Complete Mini, Roche) and phosphatase inhibitors (phosphatase inhibitor cocktail set I and II, Calbiochem). The protein concentrations of whole cell lysates were measured by NanoDrop OneC using the Bio-Rad protein assay reagent as described previously [52]. Equal amounts of whole cell lysates were resolved by SDS-PAGE and immunoblotted with indicated antibodies. For immunoprecipitations analysis, unless specified, 1000 μg lysates were incubated with the indicated antibody (1-2 μg) for 3-4 hr at 4° C. followed by 1 hr incubation with 10 μL Protein A magnetic beads (New England Biolabs). Or 1000 μg lysates containing tagged molecules were incubated with agarose beads coupled antibodies for the specific tag for 3-4 hr at 4° C. For endogenous IPs, incubation of cell lysates with antibodies was extended to overnight. The recovered immuno-complexes were washed five times with NETN buffer (20 mM Tris, pH 8.0, 100 mM NaCl, 1 mM EDTA and 0.5% NP-40) before being resolved by SDS-PAGE and immunoblotted with indicated antibodies.

### Antibodies

All antibodies were used at a 1:1000 dilution in TBST buffer with 5% non-fat milk for western blotting. anti-GST antibody (2625), anti-Cullin3 antibody (2759), anti-CD99 antibody (20992), anti-CK1 antibody (2655), anti-BRD4 antibody (13440), anti-Plk1 antibody (4513), anti-Akt-pS473 antibody (4060), anti-cleaved-caspase 3 antibody (9661), anti c-Myc antibody (5605) and anti-myc-tag antibody (2278) were obtained from Cell Signaling Technology. Anti-FLI1 antibody (ab180902), anti-NRF2 (ab62352), anti-ERG (ab92513) and anti-Ki67 antibody (ab254123) were obtained from Abcam. Anti-SPOP antibody (16750-1-AP) was purchased from Proteintech. Anti-EWSR1 antibody (A300-417) was purchased from Bethyl Laboratories. Polyclonal anti-HA antibody (sc-805), anti-p27-antibody (sc1641), anti-NRF2 antibody (sc81342), anti-ERG antibody (271048), anti-ITGAV antibody (376156), anti-COL3A1 antibody (271249) and anti-Vinculin antibody (sc-25336) were obtained from Santa Cruz Biotechnology. Polyclonal anti-Flag antibody (F-2425), monoclonal anti-Flag antibody (F-3165, clone M2), anti-Tubulin antibody (T-5168), anti-OTUD3 antibody (PA5-98487), anti-OTUD7A antibody (SABB04135), anti-Flag agarose beads (A-2220), anti-HA agarose beads (A-2095), glutathione agarose beads (G4510), peroxidase-conjugated anti-mouse secondary antibody (A-4416) and peroxidase-conjugated anti-rabbit secondary antibody (A-4914) were obtained from MilliporeSigma. Monoclonal anti-HA antibody (MMS-101P) was obtained from BioLegend.

### Generation of EWS-FLI1-3A Knockin A673 Cells

Parental A673 cells were splitted into 24-well plates and transfected with sgRNA against EWS-FLI1 together with EWS-FLI1-3A-ssoDNA following protocols as described [58]. 1-day post-transfection, cells were selected with 1 μg/ml puromycin for 3 days. Surviving cells were counted and each single cell was seeded into 96-well plates. Each single clone grown up in 96-well plates was amplified and one copy was used for genomic DNA extraction, followed by PCR and BpmI digestion to screen for potential knockin clones. BpmI negative clones are selected and sequenced to verify the knockin at the DNA level. 3 isogenic knockin clones were selected and saved.

shRNA-Mediated OTU Screen to Identify OTUs Critical to Maintain Ewing Sarcoma Growth

Three independent shRNAs against each OTU member were selected and lenti-viruses expressing each shRNA was individually packaged following protocols as described [54]. A673 cells were infected with each individual shRNA expressing lentiviruses for 24 hrs, recovered for 72 hrs before 1,000 surviving cells from each group were seeded in 96-well plates in triplicates. 72 hrs later MTT assays were performed to determine cell viability.

### Sample Preparation for Proteomic Analysis

A673 cells were treated with H₂O or 1 μg/mL tetracycline (to induce a Tet-inducible OTUD7A depletion) for 72 hr (n=3 biological replicates per time point). Cells were washed three times with ice-cold cold PBS, then lysed in 8 M urea, Tris-HCl (pH 7.6) with protease and phosphatase inhibitors (Bimake). Lysates were reduced with 5 mM DTT, alkylated with 15 mM iodoacetamide, then subjected to digestion with LysC (Wako) for 2 h then trypsin (Promega) overnight at 37 C at a 1:50 enzyme:protein ratio. The resulting peptide samples were acidified, desalted using Thermo desalting spin columns, then the eluates were dried via vacuum centrifugation. Peptide concentration was determined using Pierce Quantitative Colorimetric Peptide Assay. 40 μg of each sample was reconstituted with 50 mM HEPES pH 8.5, then individually labeled with 60 ug of TMT 10plex reagent (Thermo Fisher) for 1 hr at room temperature. Labeling efficiency was evaluated by LC-MS/MS analysis of a pooled test mix. Samples were quenched with 50% hydroxylamine to a final concentration of 0.4%. Labeled peptide samples were pooled, desalted using Thermo desalting spin column, and dried via vacuum centrifugation. The dried TMT-labeled sample was fractionated using high pH reversed phase HPLC [59]. Briefly, the sample was offline fractionated over a 90 min run, into 96 fractions by high pH reverse-phase HPLC (Agilent 1260) using an Agilent Zorbax 300 Extend-C18 column (3.5-μm, 4.6×250 mm) with mobile phase A containing 4.5 mM ammonium formate (pH 10) in 2% (vol/vol) LC-MS grade acetonitrile, and mobile phase B containing 4.5 mM ammonium formate (pH 10) in 90% (vol/vol) LC-MS grade acetonitrile. The 96 resulting fractions were then pooled in a non-continuous manner into 24 fractions. The 24 fractions were dried via vacuum centrifugation.

### LC/MS/MS Analyses

The 24 fractions were analyzed by LC/MS/MS using an Easy nLC 1200 coupled to a QExactive HF mass spectrometer (Thermo Scientific). Samples were injected onto an EASY-Spray PepMap C18 column (75 μm id×25 cm, 2 μm particle size) (Thermo Scientific) and separated over a 150 min method. The gradient for separation consisted of 5-50% mobile phase B at a 250 nl/min flow rate, where mobile phase A was 0.1% formic acid in water and mobile phase B consisted of 0.1% formic acid in 80% ACN. The QExactive HF was operated in data-dependent mode where the 15 most intense precursors were selected for subsequent HCD fragmentation. Resolution for the precursor scan (m/z 350-1600) was set to 60,000 with a target value of 3×10⁶ ions and a maximum injection time of 100 ms. MS/MS scans resolution was set to 60,000 with a target value of 1×10⁵ ions and a maximum injection time of 100 ms. Fixed first mass was set to 110 m/z and the normalized collision energy was set to 32% for HCD. Dynamic exclusion was set to 30 s, peptide match was set to preferred, and precursors with unknown charge or a charge state of 1 and >8 were excluded.

### Proteomics Data Analyses

Raw data files were processed using MaxQuant v1.6.12.0, set to ‘reporter ion MS2’ with ‘10plex TMT’. Peak lists were searched against a reviewed Uniprot human database (downloaded February 2020 containing 20,350 sequences), appended with EWS-FLI1 sequences and a common contaminants database, using Andromeda within MaxQuant. All fractions were searched with up to three missed trypsin cleavage sites, fixed carbamidomethylation (C) modification, dynamic oxidation (M), deamidation (NQ) and acetylation (N-terminal) modifications. Peptide false discovery rate was set to 1%. Data were further analyzed in Perseus and Microsoft Excel. Each reporter ion channel was summed across all quantified proteins and mean-normalized assuming equal protein loading of all samples.

RNA Extraction and iRT-PCR

Cells were isolated by dissociation with 0.05% Trypsin, followed by media quenching. The cells were spun down at 300×rcf for 5 minutes. The media was aspirated, the pellet was suspended with 1×PBS, and then the cells were spun down again. The PBS was aspirated. RNA extraction was performed with an RNA extraction kit (BioBasic BS584). The final elution step was done with 50 μL of RNAse-free water. The relative enrichment of mRNA was quantified with the NanoDrop OneC (Thermo Fisher Scientific). Three biological replicates were performed for RNA extraction. Quartile analysis was done to exclude outliers and significance was determined by one-way ANOVA tests.

### Cell Viability (MTT) Assays

2,000 indicated cells were seeded in each well of 96-well plates for MTT assays to monitor cell viability at indicated time periods using a method adapted from https://www.thermofisher.com/us/en/home/references/protocols/cell-culture/mtt-assay-protocol/vybrant-mtt-cell-proliferation-assay-kit.html. Briefly, at indicated time points post-cell seeding, 10 μL MTT solution was added into each well and incubated in the culture incubator (37° C. with 5% CO₂) for 4 hrs. Then medium was removed and 100 μL DMSO was added into each well to dissolve the formazan crystal and incubated for 10 min at 37° C. After thorough mixing, absorbance at 540 nm was measured using the BioTek Cytation 5 Cell Imaging reader.

### Colony Formation Assays

Indicated cells were seeded into 6-well plates (300 or 600 cells/well) or 6 cm dishes (500 or 1,000 cells/dish) and cultured in 37° C. incubator with 5% C02 for ˜14 days (as indicated in figure legends) until formation of visible colonies. Colonies were washed with 1×PBS and fixed with 10% acetic acid/10% methanol for 30 min, stained with 20% acidic acid/10% Methanol with 0.1% crystal violet until colonies were visibly stained. Colonies were then washed by tap water and air-dried. Colony numbers were manually counted. At least two independent experiments were performed to generate the error bars.

### Soft Agar Assays

The anchorage-independent cell growth assays were performed as described previously [52]. Briefly, the assays were preformed using 6-well plates where the solid medium consists of two layers. The bottom layer contains 0.8% noble agar and the top layer contains 0.4% agar suspended with 3×10⁴ or indicated number of cells. 500 μL complete DMEM medium with 10% FBS was added every 4 days. About 4 weeks later the cells were stained with iodonitrotetrazolium chloride (1 mg/mL) (Sigma 110406) overnight for colony visualization and counting. At least two independent experiments were performed to generate the error bar.

### Mouse Xenograft Assays

All mouse work has been reviewed and approved by UNC Institutional Animal Care and Use Committee under IACUC #19-031. Mouse xenograft assays were performed as described previously [52, 53]. Briefly, for mouse xenograft experiments, 2.5×10⁶ parental or EWS-FLI1-3A knockin A673 cells were mixed 1:1 with matrigel (Corning 354230) and injected into the flank of indicated female nude mice (NCRNU-M-M from UNC Animal Facility, 4 weeks old). Six days post-injection, when the tumors were established, 2% sucrose with or without 1 μg/mL tetracycline was supplied in water for mice and refreshed every 5 days. Tumor size was measured every two days with a digital caliper, and the tumor volume was determined with the formula: L×W²×0.52, where L is the longest diameter and W is the shortest diameter. After 22 days, mice were sacrificed, and tumors were dissected and weighed.

For 7Ai treatment in nude mice, 2.5×10⁶ A673 cells were mixed 1:1 with matrigel (Corning 354230) and injected into the flank of indicated female nude mice (NCRNU-M-M from UNC Animal Facility, 4 weeks old). Seven days post-injection, when the tumors were established, 100 μL 7Ai (25 mg/kg, dissolved in ethanol followed by the addition of sunflower oil and then sonicated at 4° C. in a water bath sonicator until 7Ai was completely dissolved) was given to mice through IP injections and this treatment was repeated every 3 days.

### Transwell Assays

1×10⁵ cells were plated in an 8.0-mm, 24-well plate chamber insert (Corning Life Sciences, catalog no. 3422) with serum free DMEM medium at the top of the insert and the same medium containing 20% fetal bovine serum (FBS) at the bottom of the insert. Cells were incubated for 24 hr and fixed with 4% paraformaldehyde for 15 min. After washing with PBS, cells at the top of the insert were scraped with a cotton swab. Cells adherent to the bottom were stained with 0.5% crystal violet blue for 60 min and then washed with double-distilled H₂O (ddH₂O). The positively stained cells were examined under the microscope.

Immunohistochemistry (IHC)

Freshly dissected xenografted tumors are immediately fixed in 10% formalin for 2 days before transferring to 80% ethanol for one day to prepare tumor blocks. 4-μm sections were cut from each of the tumor blocks by UNC TPL facility and used for IHC study. IHC was performed as described previously [52]. Normal human tissue TMA was purchased from UNC TPL facility. Human Ewing sarcoma tissues were obtained post-mortem and fixed in 10% formalin and embedded in paraffin.

### Isothermal Titration Calorimetry (ITC)

ITC measurements were performed using a MicroCal auto-iTC200 calorimeter (MicroCal, LLC) as described previously [60]. Briefly, His-tagged OTU domain of OTUD7A proteins were purified using BL21 strain upon induced by 0.3 mM IPTG when OD600 around 0.6 overnight at 16° C. Then purified human His-tagged OTU domain of OTUD7A was dialyzed against 50 mM HEPES buffer (pH 7.2) with 100 mM NaCl for overnight at 4° C. The concentration of the protein was determined by band intensity in a gel-cod staining SDS-PAGE gel. The ITC assay was carried out at 37□C. The dialyzed His-OTUD7A-OTU proteins were diluted to 40 μM in the dialysis buffer containing 4% DMSO. Then, 400 μM 7Ai, dissolved in the same dialysis buffer with 4% DMSO (50 mM HEPES buffer (pH 7.2) with 100 mM NaCl) was injected into 0.4 mL of His-OTUD7A-OTU protein in the chamber for every 180 s. The dissociation constants and thermodynamic parameters were determined by using the embedded software package Origin7 (Microcal).

### OTUD7A OTU Domain Homology Model

A homology model for OTD7A was built on the homologous protein OTUD7B, for which the catalytic OTU domain has been crystallized both in complex with a diubiquitin, monoubiquitin, and as an apo structure (PDB codes 5LRV, 5LRW, 5LRU, respectively [61]. The paper reports three sites of interest, the distal (S1) and proximal (S1′) ubiquitin-binding sites, as well as the catalytic center. Substrate recruitment was shown to be primarily driven by the S1 site. In the interest of selectivity, it is desirable to identify a small molecule binding site that is different in the two proteins. In OTUD7B, both S1′ and the catalytic site undergo significant conformational changes upon substrate binding. Furthermore, the region around the catalytic site is highly conserved between the two homologs. S1, on the other hand, has a binding pocket formed in part by two alpha helices connected by a loop that appears suitable for small molecule inhibitors and shows relatively small changes between the three different OTUD7B crystal structures. In OTUD7A, this loop (Q257-W263) is shorter, suggesting a different shape and smaller size of S1 in the OTUD7A, providing for potential ligand selectivity.

Based on these observations, we selected the S1 region of the OTUD7A model for our virtual screening campaign. The monoubiquitin-bound OTUD7B crystal structure 5LRW (after removal of the monoubiquitin) seemed best suited as a modeling template, as it is both in a ligand-bound conformation and has density for all the residues surrounding the area of interest. The homology model of OTUD7A was built using ICM Pro v3.8.7 (Molsoft L.L.C.). Like in the experimental OTUD7B structures, the long, unstructured V-loop (residues 276-300) was replaced by QPG.

### Artificial Intelligence (AI)-Based Small Molecule Virtual Screen

The virtual screen was carried out using the AtomNet neural network, the first deep convolutional neural network for structure-based drug design [46, 47]. We employed a single global AtomNet model to predict binding affinity of small molecules to a target protein. The model was trained with experimental Ki, Kd, and IC50 values of several million small-molecules and protein structures spanning several thousand different proteins, curated from both public databases and proprietary sources. Because AtomNet is a global model, it can be applied to novel binding sites with no known ligands, a prerequisite to most target-specific machine-learning models. Another advantage of using a single global model in prospective predictions is that it helped prevent the so-called model overfitting. We applied the following three-step procedure to train AtomNet models. The first step is to define the binding site on a given protein structure using a flooding algorithm [62] based on an initial seed. The initial starting point of the flooding algorithm may be determined using either a bound ligand annotated in the PDB database or crucial residues as revealed by mutagenesis studies, or identification of catalytic motifs previously reported. The second step is to shift the coordinates of the protein-ligand co-complex to a three-dimensional Cartesian system with an origin at the center-of-mass of the binding site. In order to prevent the neural network from memorizing a preferred orientation of the protein structure, we then performed data augmentation by randomly rotating and translating the protein structure around the center-of-mass of the binding site. The third step is to sample the conformations or poses of a small molecule ligand within the binding site pocket. For a given ligand, an ensemble of poses were generated, and each of these poses represented a putative co-complex with the protein. Each generated co-complex was then rasterized into a fixed-size regular three-dimensional grid, where the values at each grid point represent the structural features that are present at each point. Similar to a photo pixel containing three separate channels representing the presence of red, green, and blue colors, our grid points represent the presence of different atom types. These grids serve as the input to a convolutional neural network, and define the receptive field of the network. We used a network architecture of a 30×30×30 grid with 1 Å spacing for the input layer, followed by five convolutional layers of 32×3³, 64×3³, 64×3³, 64×3³, 64×2³ (number of filters×filter-dimension), and a fully connected layer with 256 ReLU hidden units. The scores for each pose in the ensemble were combined through a weighted Boltzman averaging to produce a final score. These scores were compared against the experimentally measured pKi or pIC50 (converted from Ki or IC50) of the protein and ligand pair, and the weights of the neural network were adjusted to reduce the error between the predicted and experimentally measured affinity using a mean-square-error loss function. Training was done using the ADAM [63] adaptive learning method, the backpropagation algorithm, and mini-batches with 64 examples per gradient step.

AtomNet can take any form of 3D protein structures determined by experimental methods including crystallography, NMR, and cryo-EM published in Protein Data Bank (PDB) format. In case of no available experimental protein structure of the target, we can use the amino acid sequence of the target to build a homology model using the most homologous protein structure as a template as described above. We identified the binding site surrounded by residues R249, W250, R251, W252, Q253, Q254, T255, Q256, Q257, K259, E261, R265, E266, W267, E269, L270, L273, E304, E305, F306, H307, P339, F340, F400 on the OTUD7A homology model.

We screened the Mcule small-molecule library version v20171018, containing 5,648,837 small organic molecules for drug discovery purchasable from the chemical vendor Mcule. The library in SMILES format was downloaded from Mcule's website (https://mcule.com/). Every compound in the library was pushed through a standardization process including the removal of salts, isotopes and ions, and conversion to neutral form; conversion of functional groups and aromatic rings to consistent representations. We then applied filters on some molecular properties including molecular weight MW between 100 and 700 Daltons, total number of chiral centers in a molecule≤6, total number of atoms in a molecule≤60, total number of rotatable bonds≤15, and only molecules containing C, N, S, H, O, P, B, halogens are allowed. Other filters such as toxicophores, Eli Lilly's MedChem Rules [64] and PAINS were also applied to remove compounds with undesirable substructures, resulting in a filtered library of 4,025,533 compounds

For each small molecule, we generated a set of 64 poses within the binding site. Each of these poses was scored by the trained model, and the molecules were ranked by their scores. We examined the top 5000 ranking compounds from which a set of 89 compounds containing diverse chemical scaffolds were selected. The selected compounds were sourced from Mcule. Of 88 available compounds, 73 passed quality control with 62 compounds having at least 90% purity measured by LCMS. Eleven compounds had purity between 78% and 90%. The compound 7Ai has a clean mass spectrum and is at 86.9% purity. After being identified as a hit, compound 7Ai was purified by HPLC and assayed again to confirm its activity.

Activity Assay for 73 Predicted Compounds from AI-Based Virtual Screen

Each compound was dissolved in DMSO with a concentration of 10 mM. The compound samples were assayed in a blinded way (chemical identities unknown to the lab researcher, with two negative control samples containing pure DMSO mixed in). A673 and SK-N-MC cells were splitted into 6 well plates and treated with each compound with a final compound concentration of 10 μM for 12 hrs. Cells were harvested and subjected to western blot analyses.

### Statistics

Differences between control and experimental conditions were evaluated by One-way ANOVA. These analyses were performed using the SPSS 11.5 Statistical Software and P<0.05 was considered statistically significant.

### Example 1

### The E3 Ligase SPOP Targets EWS-FLI1 for Ubiquitination and Degradation

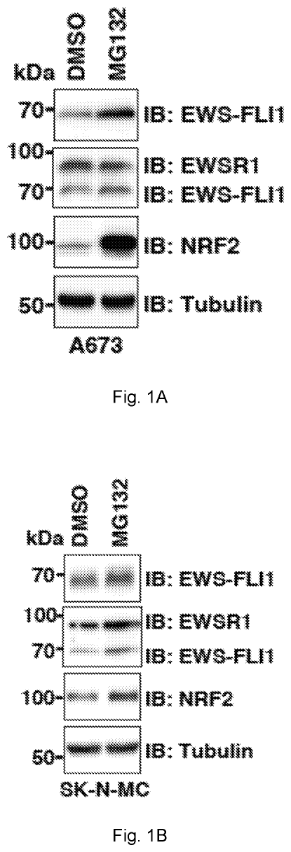

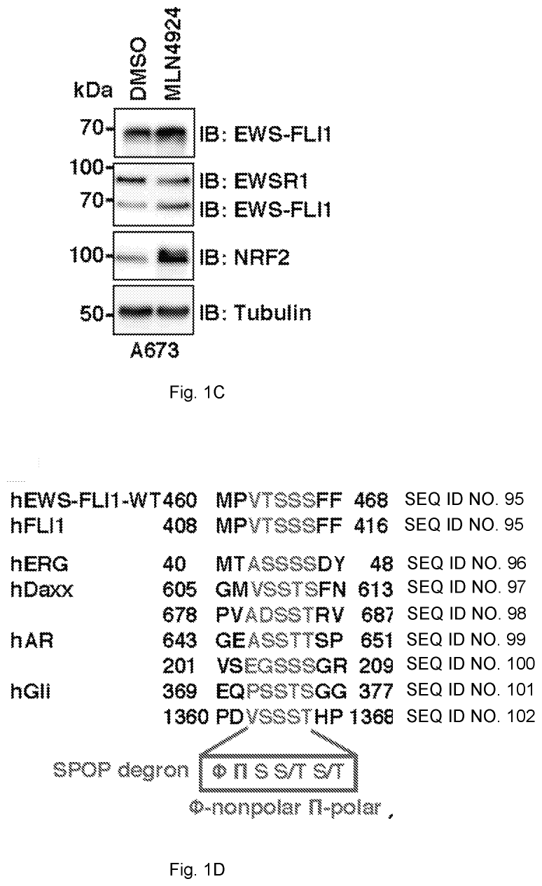

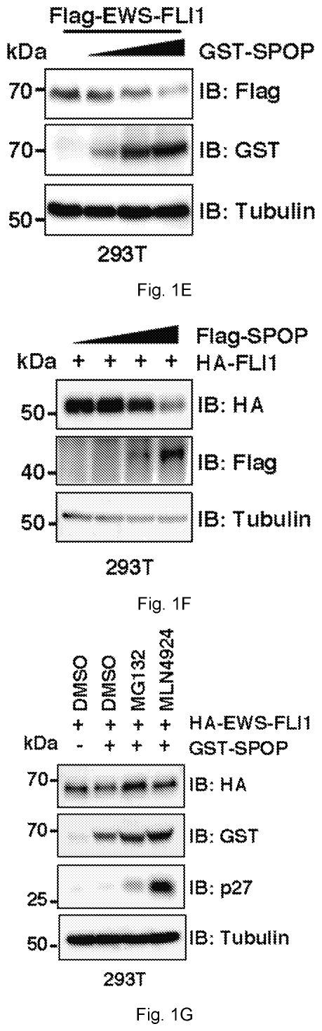

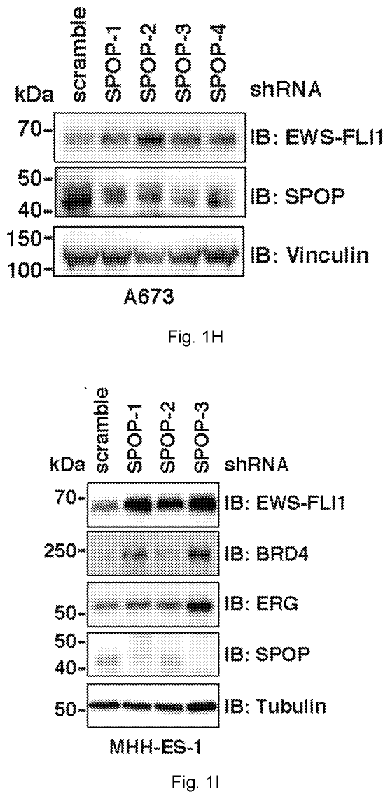

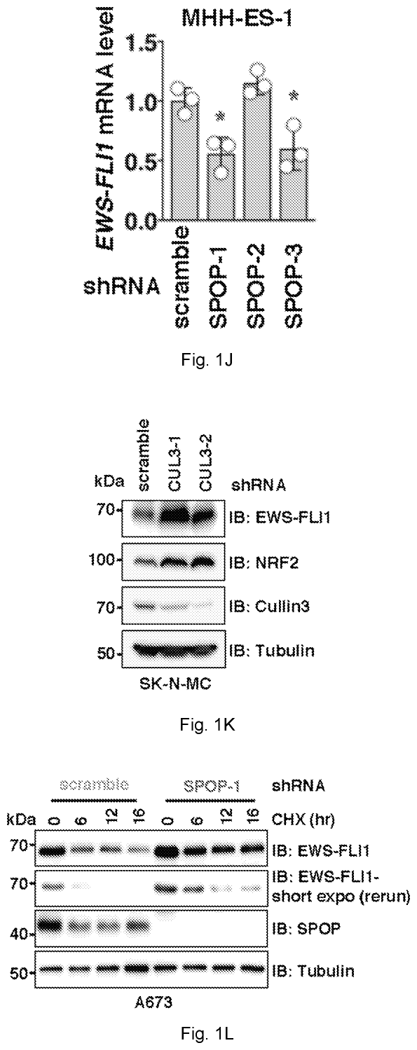

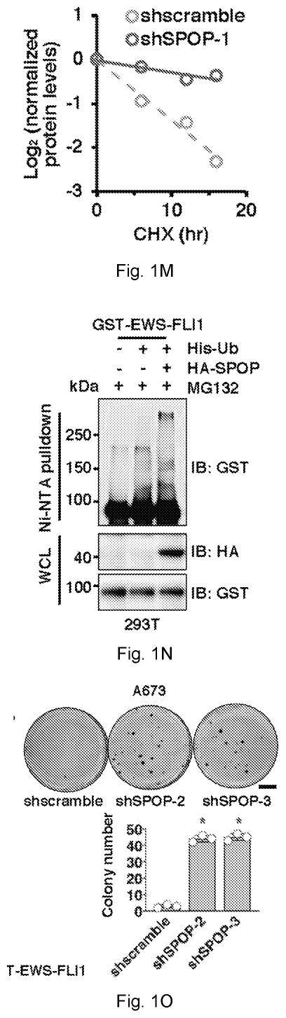

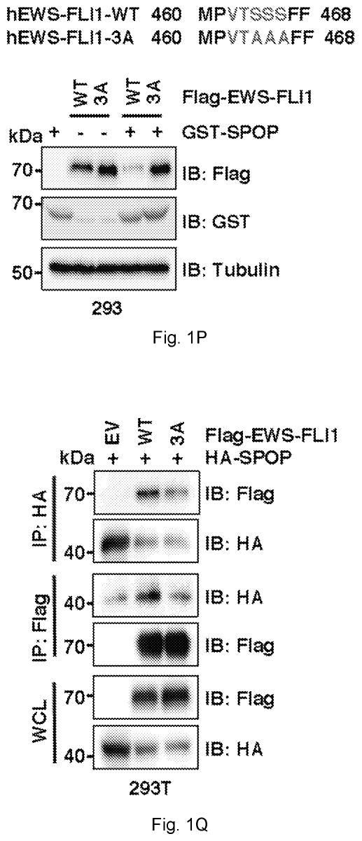

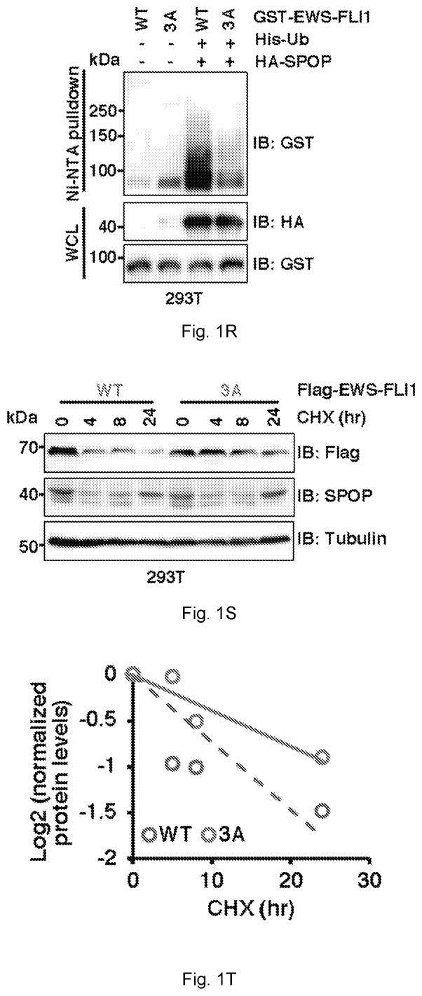

The data herein show for the first time that blocking the 26S proteasome by MG132 significantly increased EWS-FLI1 protein abundance, but not the wild-type EWSR1, in two Ewing sarcoma cell lines (A673 and SK-N-MC) (FIGS. 1A-1B), demonstrating that EWS-FLI1 levels are regulated through protein stability. Inhibition of cullin (CUL) neddylation by MLN4924 also largely stabilized EWS-FLI1 but not EWSR1-WT proteins in A673 cells (FIG. 1C). Antibodies used to detect endogenous EWSR1, FLI1 and EWS-FLI1 fusion in Ewing sarcoma cells were validated by an shRNA against FLI1-C-terminus in A673 cells. These data demonstrate that in Ewing sarcoma EWS-FLI1 protein stability is governed by CUL-Ring E3 ligases (CRL) and that the major degron resides in the FLI1-domain retained in the fusion. By examining EWS-FLI1 binding to a family of CULs, it was found that EWS-FLI1 associated with CUL3, CUL4A and CUL5. Examining the sequence of the retained FLI1 segment, a putative degron sequence (VTSSS; SEQ ID NO: 1) for SPOP was identified, a CUL3 family of E3 ligase. The sequence was located between the ETS DNA-binding domain and the carboxyl terminus (FIG. 1D). Ectopic expression of SPOP promoted EWS-FLI1 protein degradation in cells in an SPOP dose-dependent manner (FIG. 1E). Consistent with the presence of the SPOP degron in FLI1, SPOP also destabilized wild-type FLI1 but not wild-type EWSR1 (FIG. 1F). In support, SPOP bound the fusion protein but not EWSR1. Decreasing the possibility that indirect transcriptional control mediated differences in EWS-FLI1 levels, ectopically expressed EWS-FLI1 was also decreased by SPOP, and this effect was blocked by either MG132 or MLN4924 (FIG. 1G).

To further confirm SPOP as a physiological E3 ligase for EWS-FLI1, endogenous SPOP was depleted in four Ewing sarcoma cell lines (A673: FIG. 1H; MHH-ES-1: FIG. 1I; SK-N-MC: (data not shown) and EWS894: (data not shown)). In each, it was observed that SPOP depletion led to increased EWS-FLI1 protein abundance. Notably, depletion of SPOP did not increase EWS-FLI1 mRNA levels in MHH-ES-1 (FIG. 1J) or A673 cells (data not shown), supporting that SPOP regulates EWS-FLI1 largely through a post-translational mechanism. Depletion of CUL3, the cullin partner of SPOP, also increased EWS-FLI1 protein levels in SK-N-MC cells (FIG. 1K). In further support of SPOP^{CUL3} as a physiological E3 ligase for EWS-FLI1, it was observed that SPOP depletion extended the half-life of EWS-FLI1 (FIGS. 1L-1M), and SPOP expression enhanced EWS-FLI1 ubiquitination in cells (FIG. 1N). Demonstrating a functional effect in Ewing cells, it was observed that depletion of endogenous EWS-FLI1 retarded A673 cell growth in vitro, while SPOP depletion enhanced clonal proliferation of A673 cells in soft agar, an effect possibly related to increased EWS-FLI1 expression (FIG. 1O).

To test function of the putative “VTSSS” (SEQ ID NO: 1) degron in the FLI1 segment, each serine was mutated to alanine (S464A/S465A/S466A). Compared with WT-EWS-FLI1, the 3A-EWS-FLI1 mutant was resistant to SPOP-mediated degradation (FIG. 1P), largely due to deficiency of 3A-EWS-FLI1 binding to both exogenous (FIG. 1Q) and endogenous SPOP. 3A-EWS-FLI1 also displayed a resistance to MG132 treatment, reduced ubiquitination levels (FIG. 1R) and a longer protein half-life (FIGS. 1S-1T).

As SPOP has been characterized as a bonafide E3 ligase to govern BRD4 protein stability in prostate cancer [29-31], and BRD4 cooperates with EWS-FLI1 to regulates the EWS-FLI1-mediated transcriptional programs in Ewing sarcoma [32], BRD4 was examined for its involvement in SPOP depletion-induced Ewing cell growth control. To this end, it was found that SPOP depletion only moderately increased BRD4 proteins in MHH-ES-1 cells (FIG. 1I) but not in other Ewing sarcoma cells (A673 and SN-N-MC).

Moreover, given that SPOP depletion did not affect EWS-FLI1 mRNA abundance, and treatment of MHH-ES-1 or A673 cells by a BRD4 inhibitor JQ1 didn't change EWS-FLI1 protein abundance, these data demonstrate that the SPOP/BRD4 signaling axis identified in prostate cancer does not regulate EWS-FLI1 protein stability in Ewing sarcoma.

### Example 2

### CK1 Phosphorylates and Primes EWS-FLI1 for SPOP Recognition and Degradation

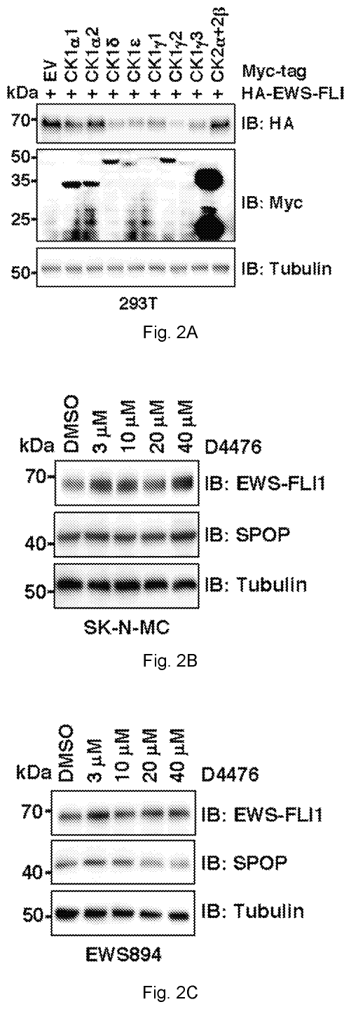

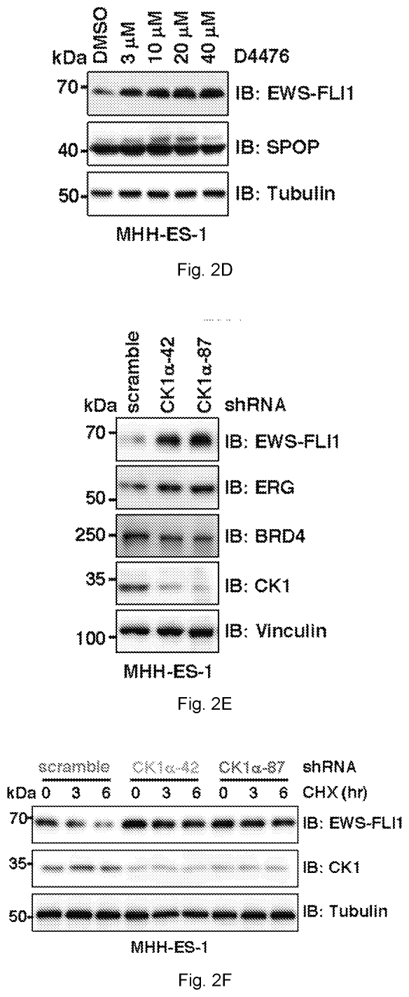

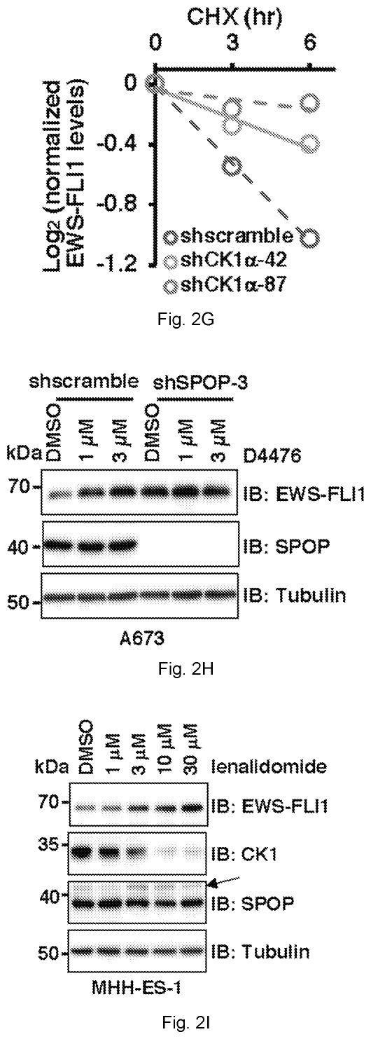

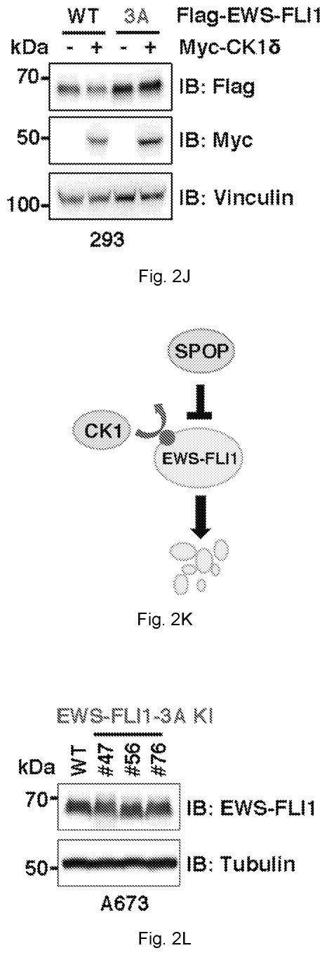

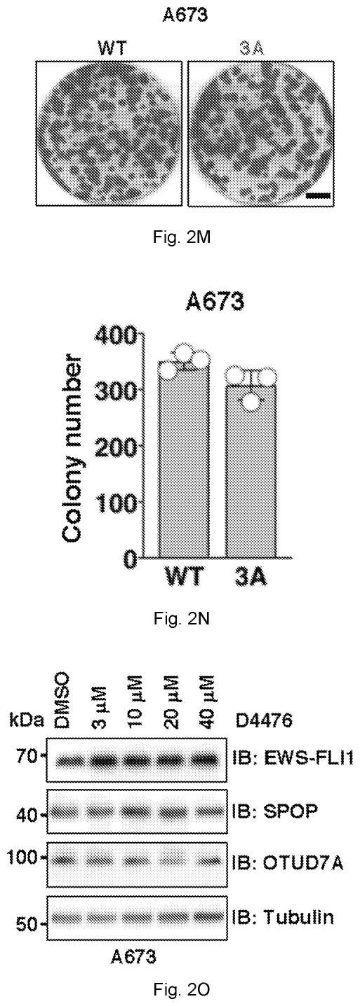

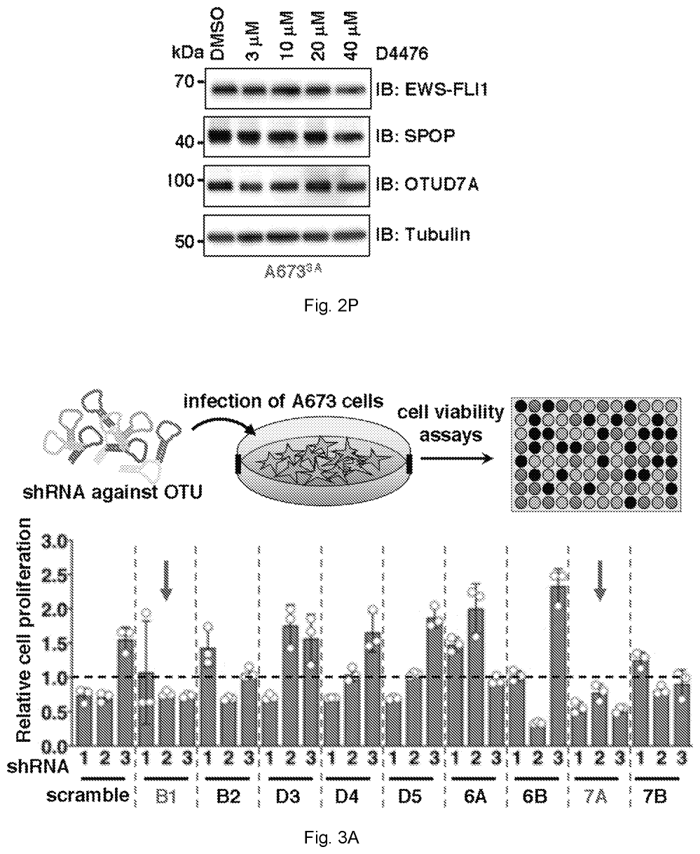

Since multiple serines in the “VTSSS” (SEQ ID NO: 1) degron could be phosphorylated, and phosphorylation of SPOP degrons can enhance SPOP-substrate binding [29, 33], experiments were next conducted to examine whether phosphorylation of the EWS-FLI1 “VTSSS” (SEQ ID NO: 1) degron primes EWS-FLI1 for SPOP recognition and degradation. Pursuing the prediction (by GPS3.0) that the serine residues could be phosphorylated by casein kinases (CKs), several distinct CK1 isoforms and CK2 kinases were expressed and it was found that most CK1 isoforms, but not CK2, promoted EWS-FLI1 degradation (FIG. 2A). Consistently, CK1 kinase inhibition with D4476 resulted in the accumulation of EWS-FLI1 in multiple Ewing sarcoma cells (FIGS. 2B-2D) without significantly affecting EWS-FLI1 mRNA levels. Similar to D4476, genetic depletion of CK1α by shRNAs also led to accumulation of endogenous EWS-FLI1 (FIG. 2E), as well as extended EWS-FLI1 half-life (FIGS. 2F-2G). Notably, SPOP levels were unaffected by D4476, and the effect of D4476 on EWS-FLI1 levels was attenuated in the context of SPOP-depletion (FIG. 2H). This attenuation was explained by reduced EWS-FLI1 binding to SPOP following D4476 treatment. In addition, lenalidomide treatment, a CK1α PROTAC that induces CK1 degradation [34], increased EWS-FLI1 protein abundance without affecting EWS-FLI1 mRNA levels (FIG. 2I). It was also found that the 3A mutant was resistant to CK1-mediated degradation (FIG. 2J), supporting Ser464/Ser465/Ser466 as functional CK1 phosphorylation sites. These data cumulatively suggest that CK1 promotes SPOP-mediated EWS-FLI1 degradation in a CK1 kinase activity-dependent manner (FIG. 2K).

Because 3A-EWS-FLI1 was resistant to both SPOP (FIG. 1P) and CK1 (FIG. 2K) mediated degradation, the degron was replaced with the 3A mutant by CRISPR mediated knock-in (KI) in A673 cells (A673^{3A}). A673^{3A} cells expressed comparable levels of EWS-FLI1 to the parental cells (FIG. 2L) and displayed a similar growth in vitro (FIGS. 2M-2N). Increased EWS-FLI1 protein levels observed in A673 parental cells upon D4476 treatment (FIG. 2O) was not observed in the A673^{3A} KI cells (FIG. 2P). In contrast to A673 cells, it was observed that neither D4476 treatment, nor SPOP depletion increased EWS-FLI1 protein abundance in EWS502 cells. Exploring genetic alterations in Ewing sarcoma cells (DEPMAP portal) it was noted that a point mutation in CUL3 (E358Q) that was present only in EWS502 cells. It was hypothesized that E358Q might result in a loss-of-function mutant such that CUL3-E358Q-containing SPOP E3 ligases in EWS502 cells cannot degrade physiological SPOP substrates. Consistent with this notion, EWS-FLI1 protein levels did not increase following CUL3 depletion in EWS502 cells. Unlike WT-CUL3, ectopic expression of E358Q-CUL3 failed to promote EWS-FLI1 degradation. Interestingly, as a scaffolding subunit in CUL3 E3 ligase complexes, the E358Q-CUL3 mutant retained a comparable binding to both SPOP and Rbx1 as WT-CUL3. In addition, E358Q-CUL3 was also efficiently neddylated to a comparable level as WT-CUL3 in cells, a modification critical for CUL3 E3 ligase activation and function [35]. These results suggest that this E358Q-CUL3 mutant forms an intact SPOP^{CUL3} E3 ligase complex. Notably, compared with WT-CUL3, E358Q-CUL3 was deficient in facilitating SPOP binding to EWS-FLI1, suggesting that the inability of SPOP^{CUL3-E358Q} E3 ligase complexes in degrading EWS-FLI1 might partly be due to that the E358Q-CUL3 mutation weakens SPOP binding to its substrates, including EWS-FLI1. This result offers an additional layer of regulation for SPOP binding to its substrates through CUL3 mutations. Together, these data further support a physiological role of SPOP^{CUL3} in targeting EWS-FLI1 for degradation and suggest that Ewing sarcoma tumors may inactivate SPOP-mediated EWS-FLI1 degradation through CUL3 mutations to promote Ewing sarcoma growth.

### Example 3

### The Deubiquitinase OTUD7A is Identified as a DUB to Control EWS-FLI1 Protein Stability Through a Genetic Screen

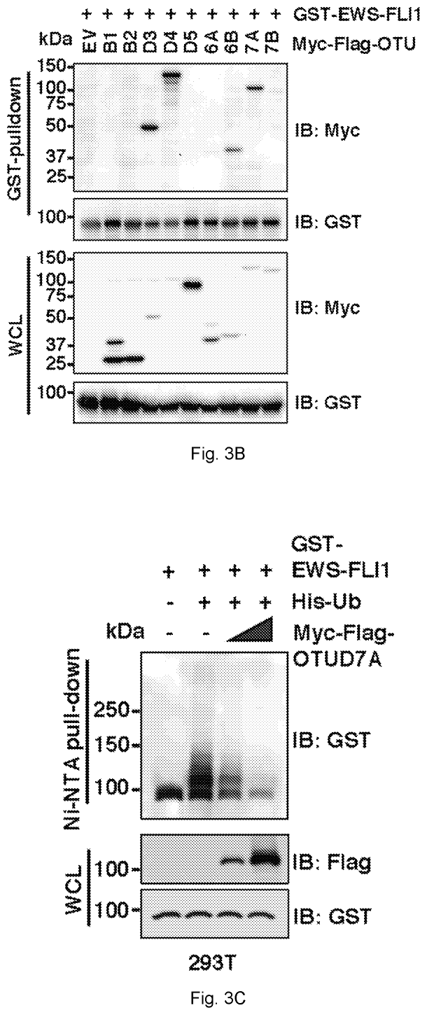

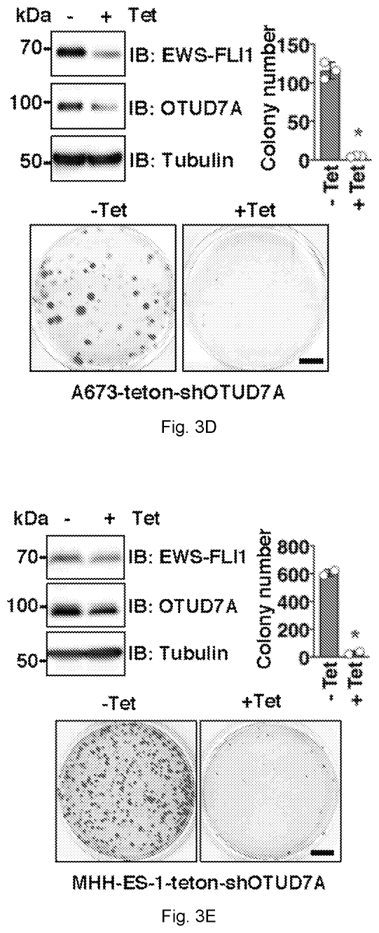

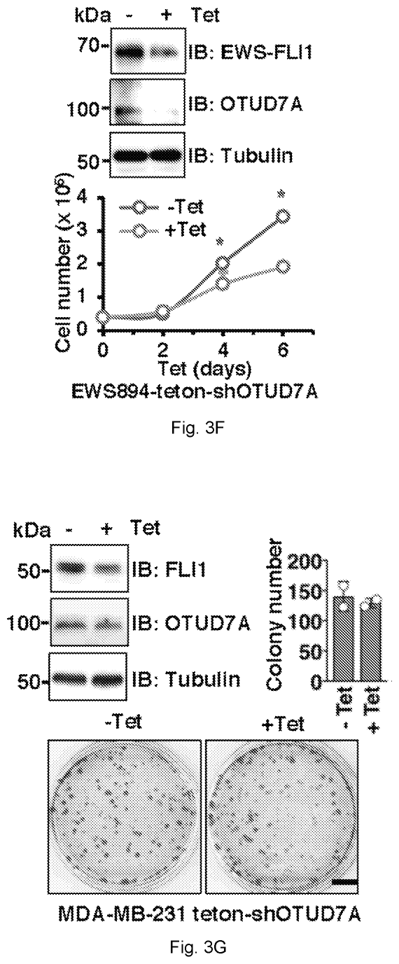

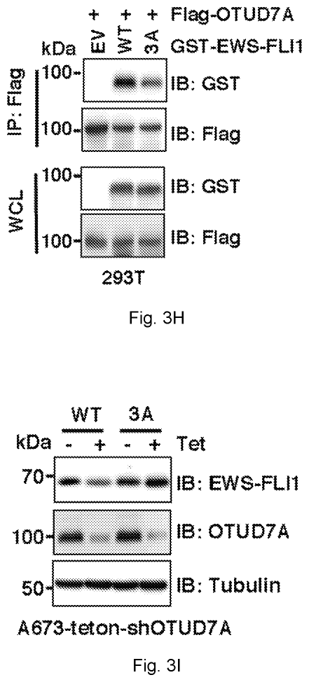

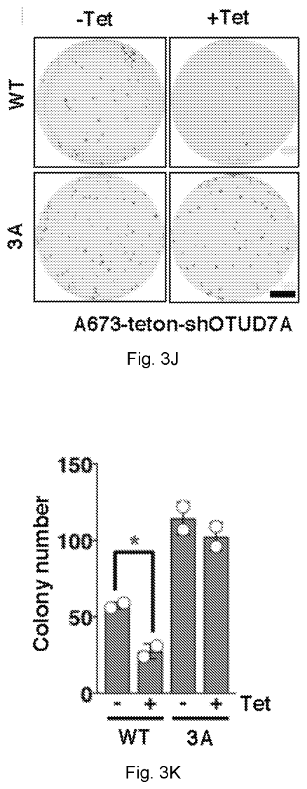

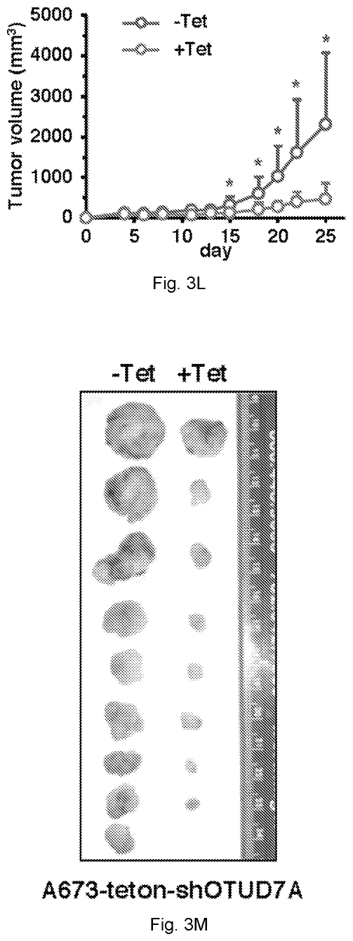

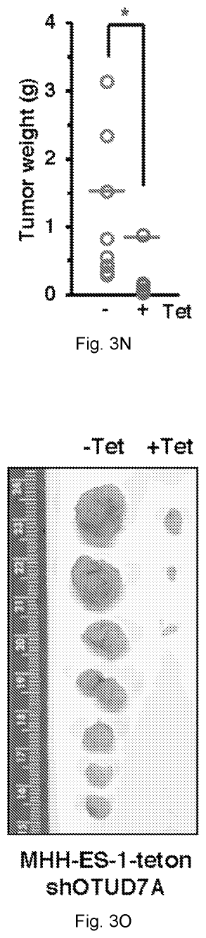

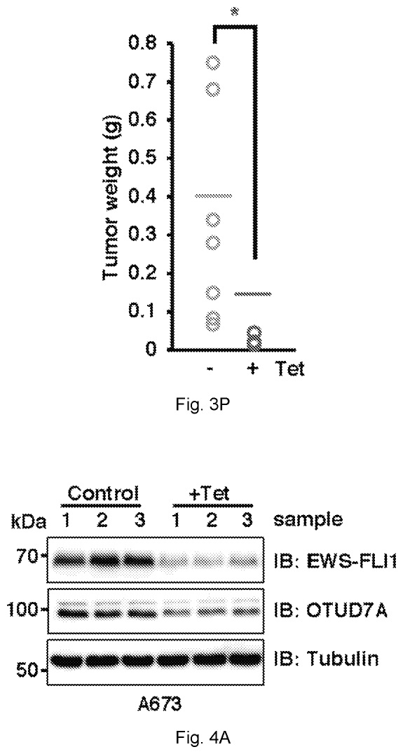

Based on the discovery that SPOP/CK1 destabilizes EWS-FLI1, activation of SPOP/CK1 could offer a therapeutic strategy to treat Ewing sarcoma. However, potential tumor suppressor functions of SPOP [33, 36] and CK1 [37], as well as the predicted challenge of targeting SPOP^{CUL3}, led to the evaluation for possible DUBs that would antagonize SPOP^{CUL3} function to stabilize EWS-FLI1. Among the five families of DUBs [38], focus was placed on ovarian tumor proteases (OTUs) since they recognize specific ubiquitin chain linkages to regulate distinct signaling cascades associated with human tumors [39]. Thus far 16 mammalian OTUs have been identified. Roles of OTUD7B in maintaining mTOR complex homeostasis [40], activating NF-kB signaling [41] and regulating the cell cycle [42] have been reported. However, the physiological roles for the majority of OTUs are just beginning to be appreciated. Since EWS-FLI1 is necessary for Ewing sarcoma growth, it was reasoned that inhibiting DUBs that stabilize EWS-FLI1 would reduce Ewing sarcoma cell proliferation by down-regulating EWS-FLI1. OTU-directed shRNAs were screened for those that decreased A673 cell proliferation. Three independent shRNAs were used to silence each of nine OTU genes. Cell viability was monitored at 3-day post-shRNA infection by MTT assays, or at 3-week after shRNA infection by colony formation assays. It was found that depletion of OTUB1 and OTUD7A reduced A673 cell growth (FIG. 3A) and diminished colony formation. Because alterations in cell growth could also result from non-EWS-FLI1 OTU targets, amined interactions between each individual OTU with EWS-FLI1 were examined next. It was discovered that OTUD3, OTUD4, OTUD6B and OTUD7A bound to EWS-FLI1 in cells (FIG. 3B). Among these 4 OTUs, ectopic expression of OTUD3 or OTUD7A, but not OTUD6B nor OTUD4, stabilized endogenous EWS-FLI1 proteins in A673 cells. For the first time, these data support OTUD3 and OTUD7A as candidates to regulate EWS-FLI1 protein stability. Consistently, both OTUD3 and OTUD7A could deubiquitinate EWS-FLI1 in cells (FIG. 3C). However, in MHH-ES-1 cells depletion of endogenous OTUD3 minimally influenced EWS-FLI1 protein abundance and did not significantly affect cell growth. Depletion of endogenous OTUD7A by shRNAs or sgRNAs led to reduced EWS-FLI1 protein abundance in SK-N-MC and A673 cells. These data support OTUD7A as a possible EWS-FLI1 deubiquitinating enzyme to control EWS-FLI1 protein stability.

### Example 4

### Genetic Depletion of OTUD7A Impedes Ewing Sarcoma Growth

Stable OTUD7A depletion led to cell death within a week of shRNA or sgRNA infection, preventing further analysis of the signaling changes and biological effects of OTUD7A loss. To overcome this, an inducible OTUD7A depletion system was developed. 48 hours post-tetracycline addition, a reduction in endogenous OTUD7A and EWS-FLI1 proteins was observed, with minimal effects on EWS-FLI1 mRNAs. Surprisingly, induced-depletion of OTUD7A led to reduced EWS-FLI1 protein levels in multiple Ewing sarcoma cells, including A673 (FIG. 3D), MHH-ES-1 (FIG. 3E) and EWS894 (FIG. 3F). MG132 treatment largely preserved EWS-FLI1 protein levels following OTUD7A depletion, further supporting a role of OTUD7A on regulating EWS-FLI1 protein stability. Importantly, in each Ewing sarcoma cell lines, OTUD7A depletion reduced cell proliferation in vitro (FIGS. 3D-3F). In contrast, depletion of endogenous OTUD7A in non-Ewing sarcoma cells such as MDA-MB-231 cells by either tet-inducible shOTUD7A (FIG. 3G) or stable OTUD7A depletion did not significantly affect cell growth in vitro, although it reduced endogenous FLI1 protein abundance (FIG. 3G).

In further support of a role for OTUD7A in EWS-FLI1 regulation, an interaction of OTUD7A with EWS-FLI1 at endogenous levels was observed. The ubiquitination-deficient 3A-EWS-FLI1 demonstrated reduced binding ability with OTUD7A (FIG. 3H), and depletion of OTUD7A failed to reduce 3A-EWS-FLI1 protein levels (FIG. 3I). These data demonstrate that OTUD7A stabilizes EWS-FLI1 proteins through the EWS-FLI1 “VTSSS” (SEQ ID NO: 1) motif and/or EWS-FLI1 ubiquitination. Thus, A673^{3A} cells offered a model to examine specific effects of inactivating the OTUD7A/EWS-FLI1 signaling. In further support of a role for inactivation of OTUD7A on impeding Ewing sarcoma proliferation, depletion of OTUD7A resulted in significantly reduced colony formation in vitro in A673^{WT} but not A673^{3A} cells (FIG. 3J-3K). Moreover, depletion of OTUD7A dramatically reduced tumor growth (FIG. 3L) and tumor formation of A673 (FIGS. 3M-3N), but not A673^{3A} cells grown as xenografts. Depletion of OTUD7A also retarded xenografted MIH-ES-1 tumor development (FIGS. 30-3P). Further histological analyses of xenografted MHH-ES-1 tumors revealed that induced depletion of OTUD7A efficiently reduced EWS-FLI1 protein levels and subsequent cell proliferation (evidenced by Ki67 staining), accompanied by increased cell death (cleaved-caspase 3). Together, these data demonstrate the dependence of Ewing sarcoma growth in vitro and in a xenografted mouse model on OTUD7A.

Although it is known that FLI1 cannot rescue the loss of EWS-FLI1 in Ewing sarcoma, a 3A mutation was detected in both EWS-FLI1 and FLI1 alleles in A673 cells. To formally demonstrate that the effect of OTUD7A is through the fusion oncoprotein, EWS-FLI1-3A expression was reconstituted through lentiviral infection into A673-teton-shOTUD7A cells and it was observed that expressed EWS-FLI1-3A was resistant to OTUD7A depletion. As a result, unlike WT-EWS-FLI1, OTUD7A depletion failed to significantly impede 3A-EWS-FLI1 expressing A673 cell growth in vitro and as a xenograft. As predicted, reconstitution of FLI1-3A expression in A673-teton-shOTUD7A cells could not rescue OTUD7A depletion induced A673 cell growth retardation in vitro. These data show that OTUD7A controls A673 cell growth largely through regulating EWS-FLI1 but not FLI1 protein stability. To further reinforce this finding, EWS-FLI1-3A expression was also reconstituted in both EWS894-teton-shOTUD7A and MHH-ES-1-teton-shOTUD7A cells and it was found that EWS-FLI1-3A expression could largely rescue OTUD7A-depletion induced growth retardation in both cell lines. Together, these data demonstrate that OTUD7A largely governs Ewing sarcoma growth by maintaining EWS-FLI1 protein stability and support prior studies demonstrating that EWS-FLI1 acts distinctly from FLI1.

### Example 5

### Quantitative Proteomics Supports EWS-FLI1 as an Endogenous OTUD7A Target and Defines a Subset of Characterized EWS-FLI1 Downstream Targets Mediating OTUD7A/EWS-FLI1 Governed Cell Growth

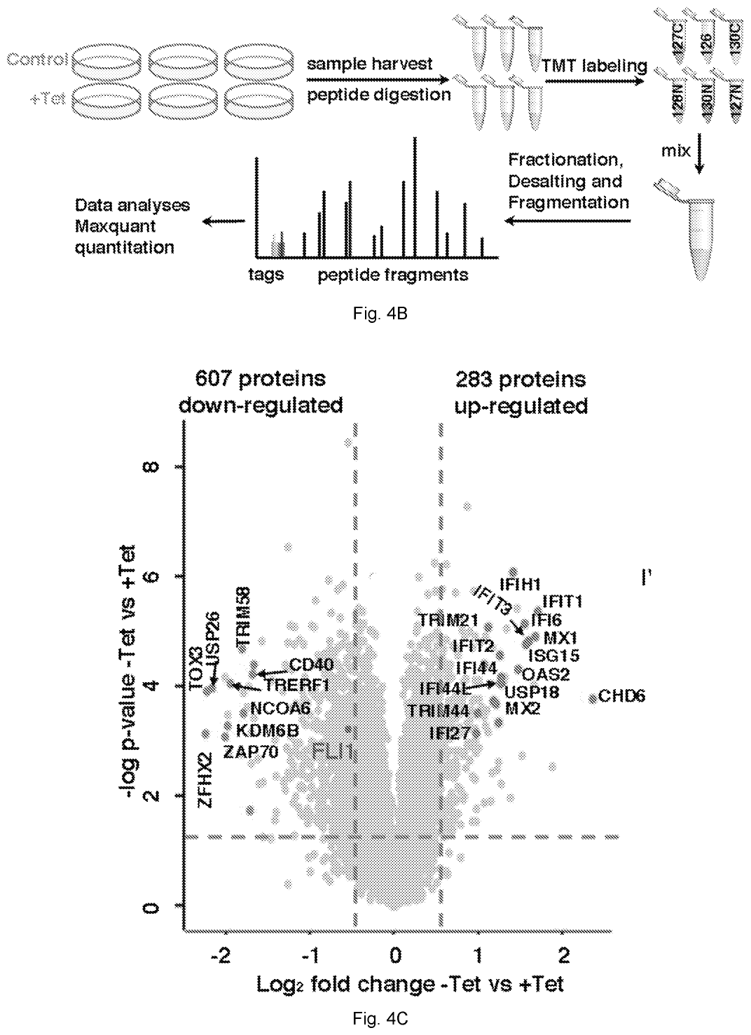

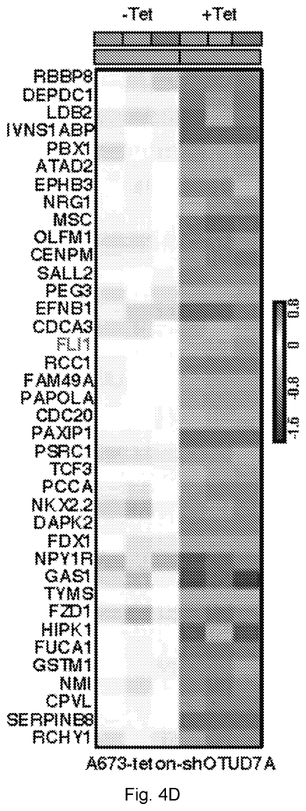

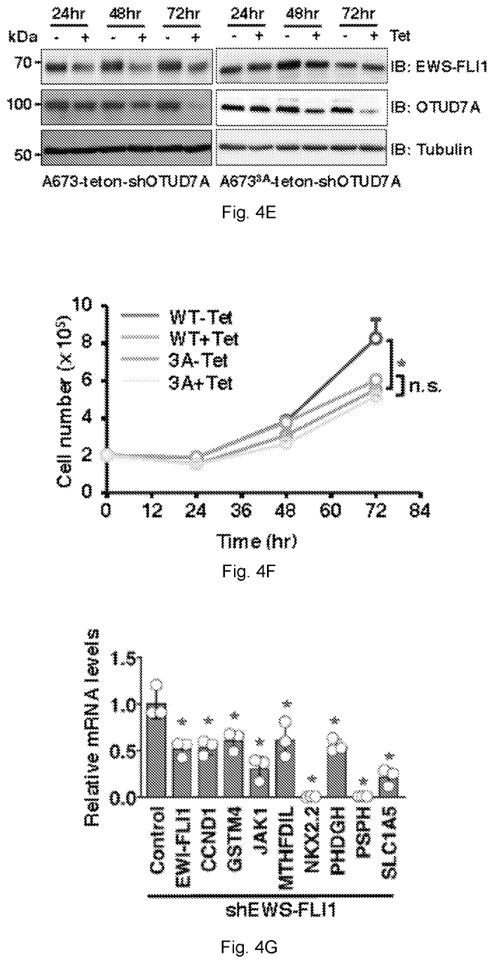

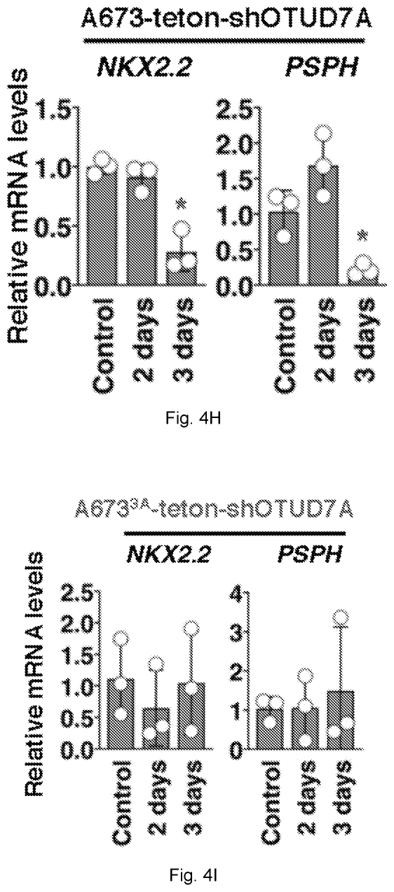

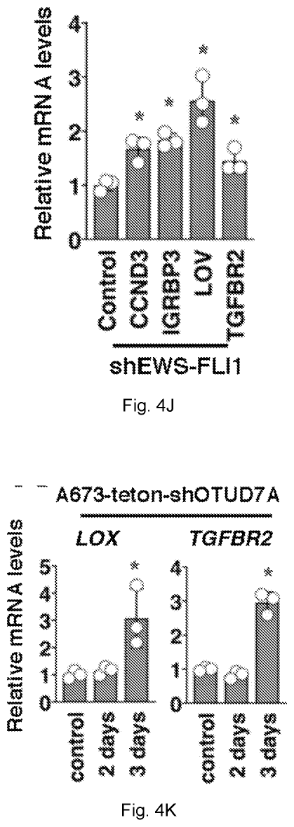

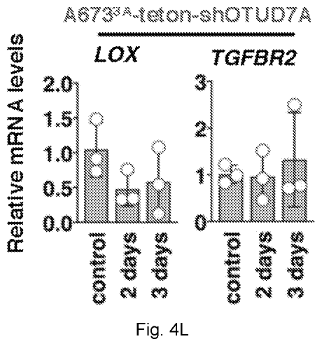

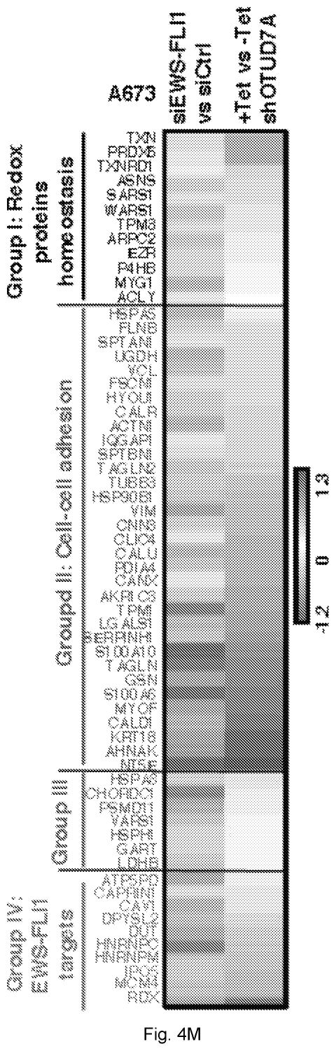

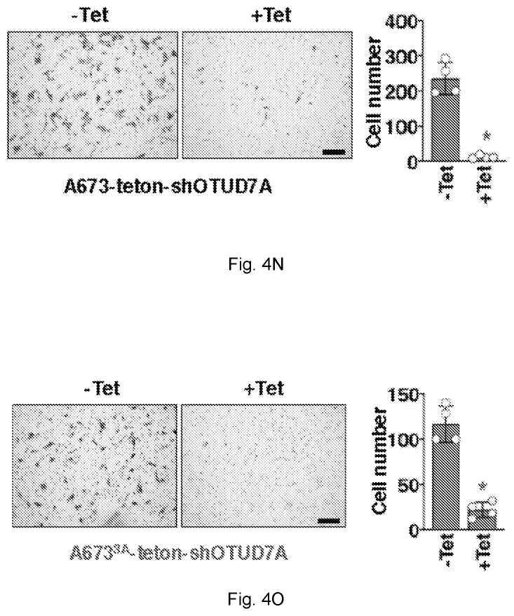

To further understand pathophysiological function of OTUD7A in Ewing sarcoma, a quantitative proteomics study was performed following genetic OTUD7A inactivation in A673 cells. 72 hrs following OTUD7A depletion by shRNA induction, significantly reduced EWS-FLI1 protein levels (FIG. 4A) were observed. At this time, proteins extracted from OTUD7A depleted (or control) cells were subjected to non-biased quantitative mass spectrometry analyses to determine differences in protein abundance (FIG. 4B). After excluding common contaminants and proteins non-specifically enriched from reported microproteins, a total of 7,641 non-redundant proteins were identified with protein abundance changes. These data constitute one of the largest Ewing sarcoma related proteomic datasets to date. Applying a p-value<0.05 and log 2 fold change>0.5 or <−0.5 threshold for differential protein abundance, it was observed that OTUD7A depletion resulted in statistically significant changes of abundance for 890 endogenous proteins, with 283 being upregulated and 607 downregulated (FIG. 4C). Notably, these proteomic data were highly reproducible among replicates within the same group. It was found that FLI1 C-terminus peptides (derived from EWS-FLI1) were significantly decreased (FIG. 4C and Table 1), a result consistent with western blot analyses (FIG. 4A). To explore whether OTUD7A depletion may modulate the abundance of proteins encoded by EWS-FLI1 transcriptional targets, the proteomic results were compared with a well-developed transcriptomic study that identified 503 EWS-FLI1 transcriptional targets [44]. It was found that 201 proteins were identified in both the disclosed proteomics study and the transcriptomic study. Among them, 33 proteins were significantly down-regulated (including EWS-FLI1, FIG. 4D and Table 1) and 6 proteins were significantly upregulated (Table 2) upon genetic OTUD7A depletion. Another 99 EWS-FLI1 transcriptional targets, demonstrated decreased levels but did not to reach statistical significance (p<0.05). These data show that OTUD7A/EWS-FLI1 signaling modulates a subset of EWS-FLI1 targets.

TABLE 1
A list of 31 defined EWS-FLI1 downstream target protein
abundance reduced by OTUD7A depletion in A673 cells

| Gene | log2 fold change |
|---|---|
| SRPK1 | −0.5 |
| CDCA3 | −0.52 |
| CDC20 | −0.52 |
| TYMS | −0.52 |
| PEG3 | −0.53 |
| TCF3 | −0.55 |
| FDX1 | −0.56 |
| FAM49A | −0.56 |
| FLI1 | −0.57 |
| ATAD2 | −0.57 |
| PSRC1 | −0.59 |
| PBX1 | −0.6 |
| PAPOLA | −0.61 |
| NRG1 | −0.62 |
| NKX2-2 | −0.67 |
| EPHB3 | −0.68 |
| PCCA | −0.73 |
| SALL2 | −0.74 |
| OLFM1 | −0.76 |
| DAPK2 | −0.77 |
| RBBP8 | −0.79 |
| FZD1 | −0.8 |
| CENPM | −0.81 |
| RCC1 | −0.84 |
| DEPDC1 | −0.86 |
| LDB2 | −0.88 |
| PAXIP1 | −1.08 |
| MSC | −1.09 |
| NPY1R | −1.1 |
| EFNB1 | −1.1 |
| HIPK1 | −1.11 |
| IVNS1ABP | −1.24 |
| GAS1 | −1.56 |

TABLE 2
A list of 6 defined EWS-FLI1 downstream target protein
abundance increased by OTUD7A depletion in A673 cells

| Gene name | Log2 tet vs control |
|---|---|
| SERPINB8 | 0.72 |
| FUCA1 | 0.64 |
| NMI | 0.60 |
| CPVL | 0.56 |
| GSTM1 | 0.52 |
| RCHY1 | 0.51 |

In addition to characterized EWS-FLI1 target proteins whose protein abundance was controlled by OTUD7A (FIG. 4D), there were additional 572 proteins downregulated by OTUD7A genetic depletion with no established connection with EWS-FLI1, showing that they are potential targets for OTUD7A or uncharacterized EWS-FLI1 targets. Further DAVID analyses led to identification of enriched biological functions for these hits by plotting the -log p-value against log 2 enrichment (Table 3 and Table 4). Consistent with EWS-FLI1 signaling being a major OTUD7A downstream effector, more than one tenth (62) of the down-regulated proteins exert DNA-binding transcription activity, many of which have been well characterized to bind EWS-FLI1 on chromatin, including CBP (CREB-binding protein), forkhead box proteins, c-myc and zinc finger proteins.

TABLE 3
Top enriched functions for down-regulated
proteins in OTUD7A-depleted A673 cells

| | | | Fold |
|---|---|---|---|
| | | | En- |
| Term | Count | p-value | richment |
| Disulfide bond | 114 | 3.01E−25 | 2.77 |
| Transcription factor activity, sequence- | 62 | 1.93E−10 | 2.36 |
| specific DNA binding | | | |
| Homeobox | 21 | 7.90E−10 | 5.09 |
| Immunoglobulin-like fold | 35 | 1.51E−09 | 3.17 |
| Extracellular matrix organization | 25 | 1.83E−09 | 4.13 |
| Epidermal growth factor-like domain | 17 | 4.44E−07 | 4.43 |
| Protein digestion and absorption | 11 | 1.32E−06 | 6.72 |
| Insulin-like growth factor binding | 13 | 3.04E−06 | 5.08 |
| protein, N-terminal | | | |
| High mobility group (HMG) box | 13 | 8.98E−06 | 4.65 |
| domain | | | |
| Integrin complex | 6 | 3.10E−04 | 8.31 |

TABLE 4
Top enriched functions for up-regulated
proteins in OTUD7A-depleted A673 cells

| | | | Fold |
|---|---|---|---|
| Term | Count | p-value | Enrichment |
| Antiviral defense | 29 | 8.83E−23 | 11.31 |
| Type I interferon signaling pathway | 21 | 5.96E−20 | 15.66 |
| Innate immunity | 25 | 1.68E−13 | 6.6 |
| Response to virus | 16 | 3.93E−09 | 7.01 |
| 2′-5′-oligoadenylate synthetase | 4 | 1.69E−04 | 28.2 |
| activity | | | |
| RIG-I-like receptor signaling pathway | 8 | 9.57E−04 | 4.88 |
| Thiol protease | 11 | 0.001573 | 3.33 |
| Response to cytokine | 8 | 0.003901 | 3.92 |
| Mitophagy | 6 | 0.004002 | 5.52 |
| Nucleophagy | 16 | 0.007451 | 2.14 |

### Example 6

### Genetic OTUD7A Inactivation Reduces Expression of EWS-FLI1 Transcriptional Targets

To further confirm that the decreased protein levels for a subset of characterized EWS-FLI1 transcription targets following OTUD7A depletion (FIG. 4D) were regulated through the OTUD7A/EWS-FLI1 signaling, mRNA abundance was examined. Reduced EWS-FLI1 protein was observed 2 days following OTUD7A shRNA induction (FIG. 4E). No significant cell growth changes were observed 2-day post-Tet induction (FIG. 4F). Differences in cell proliferation were detected 3-days following OTUD7A depletion, one day following the decrease in EWS-FLI1. Without being bound by any particular theory or mechanism of action, difference may result from a lag in the downregulation of EWS-FLI1 targets. To test this possibility, mRNAs were extracted from both A673^{WT} and A673^{3A} cells 2 days and 3 days following tetracycline addition. A673 cells depleted of endogenous EWS-FLI1 by shRNAs served as a control. EWS-FLI1 depletion led to reduced EWS-FLI1 mRNA levels, as well as downregulation of known EWS-FLI1 target genes, with NKX2.2 and PSPH being the most significantly affected targets (FIG. 4G). 3 days of treatment resulted in greater suppression of these targets (FIG. 4H), an effect not detected in A673^{3A} cells (FIG. 4I). EWS-FLI1 depletion also increased expression of targets negatively regulated by EWS-FLI1 including LOX and TGFBR2 (FIG. 4J). OTUD7A depletion for 3 days, but not 2 days, led to significantly increased LOX, TGFBR and other EWS-FLI1 transcripts in parental A673 (FIG. 4K) but not A673^{3A} cells (FIG. 4L). These data support that changes in EWS-FLI1 following OTUD7A depletion affect a subset of EWS-FLI1 transcriptional targets.

### Example 7

### Quantitative Proteomics Identifies OTUD7A Downstream Targets Mediating Ewing Sarcoma Cell Migration

The proteomic results were compared with a previous proteomic study that identified siEWS-FLI1 silenced protein abundance changes [45], and found the disclosed analysis covered 103 out of 105 differentially expressed proteins controlled by EWS-FLI1 determined in [45], among which 65 were significantly changed upon OTUD7A depletion (FIG. 4M). Surprisingly, among 33 proteins up-regulated by siEWS-FLI1 but down-regulated by OTUD7A depletion, 12 were associated with cell-cell adhesion (Group II in FIG. 4M). Consistent with previous reports showing EWS-FLI1 depletion reduces Ewing sarcoma proliferation but enhances Ewing sarcoma motility [10], it was found that EWS-FLI1 depletion increased A673 cell migration in vitro. Surprisingly, consistent with the reduced expression of cell-cell adhesion proteins, OTUD7A depletion significantly reduced cell migration in vitro, in both A673^{WT} and A673^{3A} cells (FIGS. 4N-4O). These data demonstrate that OTUD7A may govern Ewing sarcoma migration independent of EWS-FLI1. Further analysis of the proteomic data identified protein candidates related to cell migration that were decreased upon depletion of OTUD7A but increased upon EWS-FLI1 depletion, including integrins and collagens such as ITGAV and COL3A1. Depletion of EWS-FLI1 increased expression of ITGAV and COL3A1, whereas OTUD7A depletion significantly reduced levels of both proteins. Interestingly, putative SPOP degrons were identified in ITGAV and COL3A1, suggesting that, similar to EWS-FLI1, OTUD7A may cooperate with SPOP to regulate these proteins, through which OTUD7A regulates Ewing cell migration. These data cumulatively suggest that OTUD7A inactivation not only impedes Ewing cell growth largely through reduced EWS-FLI1 protein stability, but also inhibits Ewing sarcoma motility through an EWS-FLI1-independent manner, possibly by regulating the levels of cell-cell adhesion related proteins. These results provided the motivation to search for potential small molecules that would inhibit OTUD7A catalytic activity as a possible therapeutic strategy for Ewing sarcoma.

### Example 8

### OTUD7A is Expressed Across Tissues, Including Ewing Sarcoma Tumors

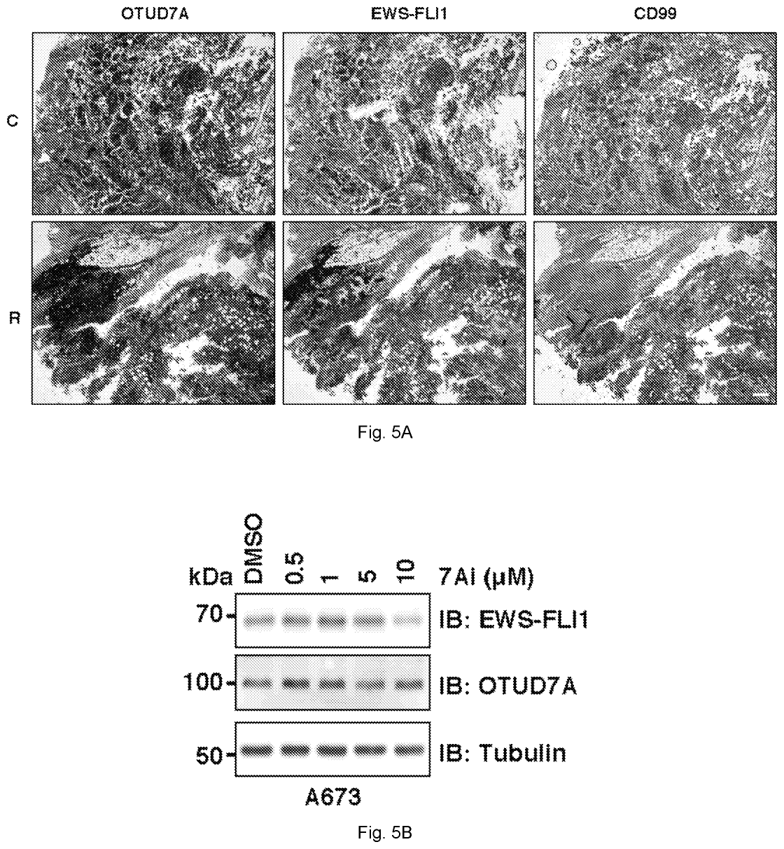

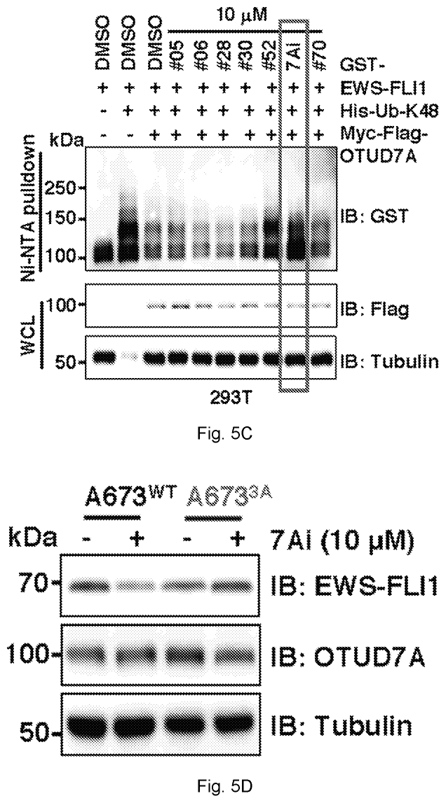

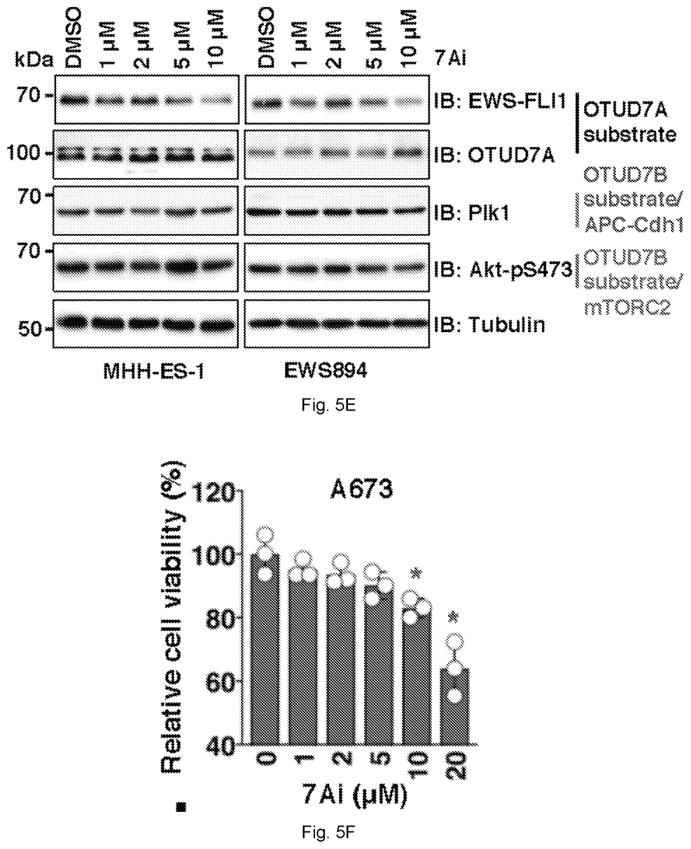

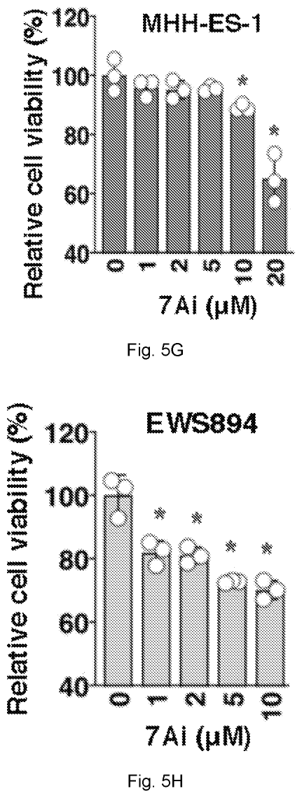

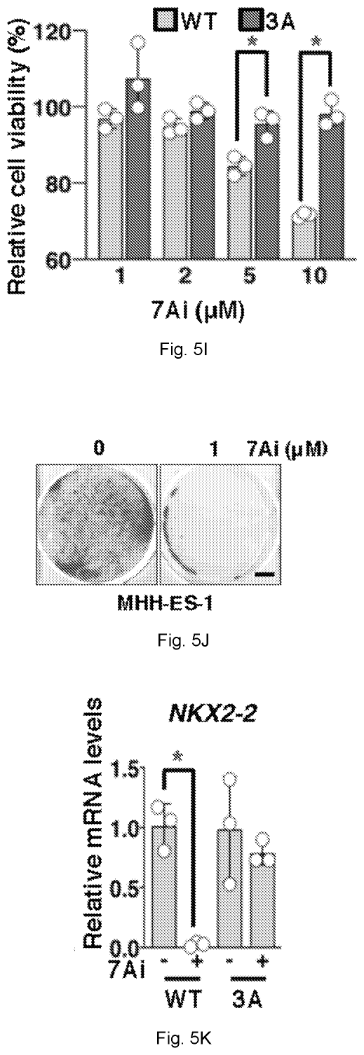

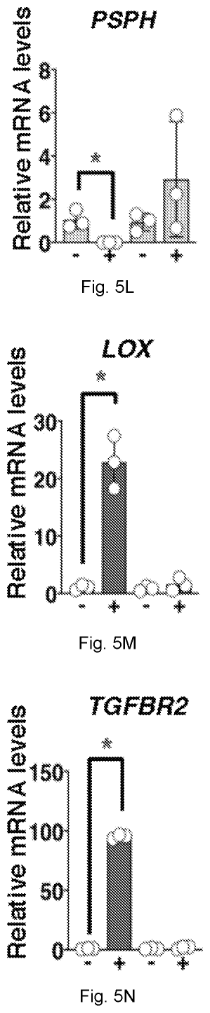

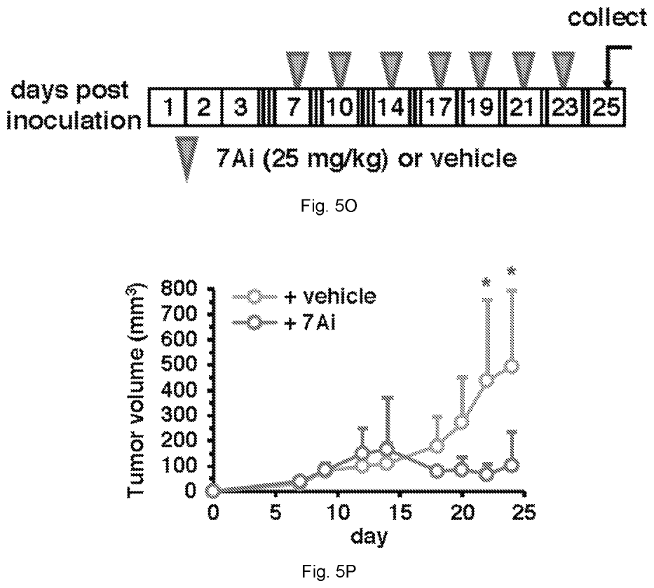

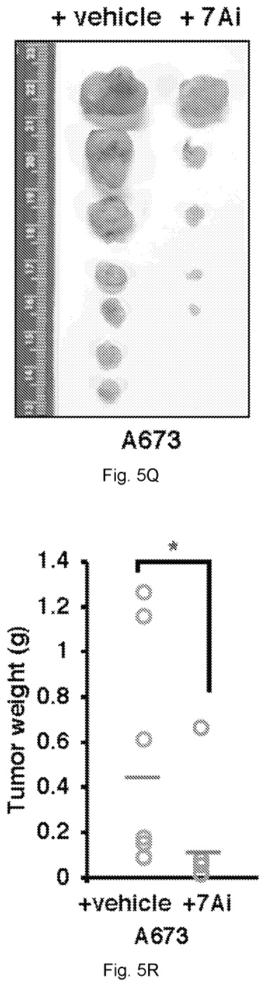

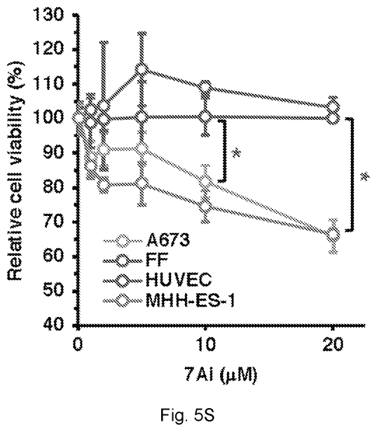

Next the therapeutic potential of inhibiting OTUD7A in treating Ewing sarcoma was examined. The data demonstrate that, in contrast to WT, the catalytic-dead C210S-OTUD7A did not stabilize EWS-FLI1. This result confirmed dependence on the OTUD7A deubiquitinase activity to regulate EWS-FLI1 protein stability. Next, the expression of UD7A proteins was profiled in a panel of commonly used Ewing sarcoma cell lines commonly used in labs and identified that all Ewing sarcoma cells expressed detectable OTUD7A. Evaluation of transcriptomic data for Ewing sarcoma cell lines and Ewing sarcoma tumors also revealed levels of OTUD7A mRNA expression. Following validation of an OTUD7A antibody, a human normal tissue TMA (tissue microarray) was assayed and varied expression levels of OTUD7A were observed among tissues largely consistent with IHC staining of tissues provided by Protein Atlas. In addition, expression of OTUD7A in mouse tissues including brain and spleen was observed. Expression of OTUD7A was also reported in different cancer types by Protein Atlas. Importantly, OTUD7A expression in metastatic Ewing sarcoma tumors was observed. OTUD7A corresponded to tumor cells, identified by CD99 and FLI1 antibody staining (FIG. 5A). These data demonstrate that OTUD7A is expressed in Ewing sarcomas and that the enzymatic activity offers a therapeutic target.

### Example 9

### Artificial Intelligence-Aided Virtual Drug Screen Identified 7Ai as an OTUD7A Catalytic Inhibitor

To rapidly assess the binding ability of drug-like small molecules to OTUD7A, AtomNet was used. AtomNet is a structure-based deep convolutional neural network virtual screening technology developed by Atomwise Inc. [46, 47]. In the absence of a published crystal structure of the OTUD7A-OTU domain, a homology model of the OTUD7A-OTU domain was first generated based on the available crystal structure of the closely related OTUD7B-OTU domain (PDB: 5LRW, 79% sequence identity in this region). Using this generated structure, a virtual screen was performed by sifting through a library of 4 million commercially available, drug-like compounds that yielded a chemically diverse set of 73 high-scoring predicted hits. These compounds were evaluated for their ability to reduce EWS-FLI1 protein abundance in both A673 and SK-N-MC cells. One compound termed as 7Ai, ranking 44^{th} out of 4,025,533 compounds that were screened, reduced EWS-FLI1 protein levels in both Ewing cells without affecting OTUD7A protein levels. Moreover, 7Ai reduced EWS-FLI1 protein levels in a dose-dependent manner within 12 hrs (FIG. 5B). Importantly, this activity was not lost following HPLC purification, suggesting that 7Ai, rather than contaminants from the chemical synthesis process, mediates OTUD7A suppression. In addition, 7Ai efficiently blocked OTUD7A-mediated deubiquitination of EWS-FLI1 in cells (FIG. 5C). Consistent with the genetic OTUD7A depletion, 7Ai reduced EWS-FLI1 protein abundance in parental A673 but not A673^{3A} (FIG. 5D), highlighting the importance of the OTUD7A/EWS-FLI1 signaling in mediating 7Ai function. Without being bound by any particular theory or mechanism of action, 7Ai did not interfere with OTUD7A binding to EWS-FLI1 which suggests that this compound might suppress OTUD7A catalytic activity through interaction with the catalytic domain. To explore whether 7Ai directly binds OTUD7A, the bacterially produced His-tagged OTUD7A OTU domain (aa183-449) was purified. Using ITC, it was demonstrated that a binding affinity of 7Ai in vitro of about 1.1 μM (stoichiometry is about 1:1). 7Ai efficiently reduced EWS-FLI1 protein expression in multiple Ewing sarcoma cells in addition to A673, including MIH-ES-1, EWS894 (FIG. 5E), SK-N-MC and EWS502. Notably, 7Ai treatment did not significantly affect OTUD7B activities as indicated by negligible changes in known OTUD7B substrates, including mTORC2 [40] and APC/Cdh1 [42] (FIG. 5E). These data support that compound 7Ai suppresses OTUD7A activity to destabilize EWS-FLI1.

### Example 10

### 7Ai Impedes Ewing Sarcoma Growth In Vitro and In Vivo

Next, the effects of 7Ai treatment on Ewing sarcoma growth were evaluated. 3-day treatment with 7Ai reduced proliferation of A673, MHH-ES-1 and EWS894 cells (FIGS. 5F-5H), which was associated with reduced EWS-FLI1 protein abundance (FIG. 5E). Notably, this effect was not observed in A673^{3A} cells (FIG. 5I). 7Ai also reduced EWS-FLI1 protein abundance in A673-teton-shOTUD7A but not same cells reconstituted with EWS-FLI1-3A. Importantly, 7Ai treatment failed to significantly suppress growth of A673 cells expressing EWS-FLI1-3A. 7Ai treatment reduced transcription of EWS-FLI1 target genes (NKX2.2 and PSPH, FIGS. 5K-5L), and increased transcription of genes negatively regulated by EWS-FLI1 (LOX and TGFBR2, FIGS. 5M-5N). 7Ai did not affected EWS-FLI1 mRNA levels. Importantly, 2-week treatment of 7Ai led to reduced colony formation ability of MMH-ES-1 (FIG. 5J) and A673 cells in vitro. Cumulatively, these data support that 7Ai suppresses Ewing sarcoma growth by reducing EWS-FLI1 protein stability.

The effect of 7Ai on xenografted tumor growth was then examined. A673 cells were subcutaneously implanted into nude mice. Once the first tumor reached about 0.5 cm in diameter, vehicle or 7Ai (25 mg/kg, IP) or vehicle were repeatedly administered every 2-3 days (FIG. 5O). Compared with vehicle control group, 7Ai treatment significantly reduced tumor volume (FIG. 5P) and tumor growth (FIGS. 5Q-5R). Notably, 7Ai administration over the 3-week treatment period did not significantly affect body weight. 7Ai treated tumors demonstrated reduced EWS-FLI1 protein and cell proliferation (Ki67 staining) and increased apoptosis (cleaved-caspase3 staining). In vitro, 7Ai treatment significantly reduced A673 cell migratory ability. These data indicate that 7Ai suppresses Ewing sarcoma growth and migration.

To further examine if 7Ai exerts a selectivity in eradicating Ewing sarcoma cells, two Ewing sarcoma cell lines A673 and MHH-ES-1, and two normal control cell lines HUVEC and foreskin fibroblast (FF) were treated in parallel with doses of 7Ai for 3 days in vitro. 7Ai treatment efficiently reduced EWS-FLI1 protein abundance in both Ewing sarcoma cells but had minimal effects on FLI1 proteins in HUVEC and FF cells. As observed with A673, 7Ai treatment reduced MHH-ES-1 proliferation but exerted neglectable effects in HUVEC and FF cells (FIG. 5S). The limited in vivo side-effect profile and effect on non-Ewing sarcoma cells provides a therapeutic window for Ewing sarcoma treatment. Subsequent formal in vivo studies will be necessary to support this observation.

### Example 11

### OTUD7A Might Also Control EWS-ERG Fusion Protein Stability in Ewing Sarcoma

In addition to EWS-FLI1 fusion observed in about 85% Ewing sarcoma tumors, other fusions including EWS-ERG (about 10% of patients) and EWS-FEV (about 1% of patients) have also been observed. Different translocation breakpoints result in Type I and Type II fusions. Our data suggest that SPOP/CK1 and OTUD7A regulate both Type I and Type II fusions, as the SPOP degron is present in both fusion types. Moreover, as predicted by the presence of the SPOP degron in the ERG fusion segment in EWS-ERG fusion, SPOP also targeted EWS-ERG for degradation, and OTUD7A stabilized EWS-ERG. EWS-ERG could partially replace EWS-FLI1 in A673 cells to maintain cell growth in vitro. In this setting, SPOP depletion stabilized EWS-ERG, and OTUD7A depletion reduced EWS-ERG that reduced cell growth. 7Ai treatment reduced EWS-ERG protein levels. Cumulatively, these data support that the vast majority of Ewing sarcoma would be targets of OTUD7A-directed treatment. More broadly, this project offers a strategy to therapeutically target a critical oncoprotein initiated by the recognition of a putative protein degron sequence.

### Discussion of Results from Examples 1-11

Because it is indispensable for Ewing sarcoma growth, the EWS-FLI1 fusion oncoprotein offers a specific therapeutic strategy as elucidated herein. Reported herein is the identification of a pathophysiologically relevant protein control mechanism. SPOP is the first E3 ubiquitin ligase that targets EWS-FLI1 for ubiquitination and degradation in a CK1 phosphorylation-dependent manner. The deubiquitinase OTUD7A antagonizes SPOP function to stabilize EWS-FLI1, revealing OTUD7A as a new Ewing sarcoma growth-dependent gene. Applying quantitative proteomic analyses, it was confirmed herein that EWS-FLI1 is a bona fide OTUD7A substrate and additional OTUD7A substrates were identified that may mediate cellular motility, independent of EWS-FLI1. Since genetic inactivation of OTUD7A reduced Ewing sarcoma proliferation and motility, OTUD7A was targeted. Using AI-aided virtual drug screening, identified herein is the first OTUD7A catalytic inhibitor, which limits Ewing sarcoma growth in vitro and in mice by degrading EWS-FLI1.

It has been reported that USP19 recognizes the EWSR1 segment to deubiquitinate and destabilize EWS-FLI1 [48]. Since EWSR1 is broadly expressed across normal cells, targeting this protein offers the potential for off-target activities. In contrast, the FLI1 domain in EWS-FLI1 is targeted by SPOP and OTUD7A. FLI1 has tissue restricted expression and deficiency is associated with thrombocytopenia in humans and mice. Although FLI1 is not broadly considered an essential gene for cell proliferation (such as MDA-MD-231 (FIG. 3G) with exceptions in certain cancers such as blood and kidney cancer. In support of this association, Tet-induced depletion of OTUD7A in kidney cancer (ACHN) and leukemia (Jurkat and CUTLL1) cells led to reduced expression of endogenous FLI1 accompanied by reduced cell proliferation. Like genetic OTUD7A depletion, pharmacological inhibition of OTUD7A by the compound 7Ai also led to reduced cell proliferation of Jurkat cells. Therefore, in addition to Ewing sarcoma, targeted inhibition of OTUD7A may demonstrate activity for also cancer types marked by FLI1 overexpression or dependent on FLI1 for proliferation, such as leukemia and kidney cancer.

Notably, due to the lack of a large cohort of patient data in Ewing sarcoma as a rare cancer, analyzing TCGA sarcoma dataset revealed that OTUD7A gene was less frequently altered. Moreover, OTUD7A mRNA expression didn't predict overall sarcoma patient survival, while if OTUD7A protein abundance predicts Ewing sarcoma patient survival remains to be further determined. Interestingly, high OTUD7A mRNA levels were associated with worse patient survival in thymoma, uterine corpus endometrial carcinoma and esophageal squamous cell carcinoma patient cohorts, and almost statistically significantly predicted worse breast cancer survival. On the contrary, high OTUD7A mRNA expression was not associated with survival of ovarian and stomach cancers, but correlated with improved survival of cervical cancer patients. These data indicate a prognostic value for OTUD7A expression in various cancers, which highlights the ability to apply 7Ai in cancers that unfavor reduced OTUD7A expression.

The instant disclosure demonstrates that both genetic and pharmacological inactivation of OTUD7A impede not only Ewing sarcoma growth but also metastasis. This is achieved by OTUD7A in controlling Ewing sarcoma growth largely through regulating EWS-FLI1 protein stability, meanwhile governing Ewing sarcoma motility, possibly through EWS-FLI1-independent substrates such as ITGAV and COL3A. Thus, inhibiting OTUD7A suppresses both Ewing sarcoma proliferation and its ability to disseminate. Whole-animal OTUD7A deletion in mice led to decreased dendritic spine density that mimicked neurodevelopmental disorders [49] associated with 15q13.3 microdeletion syndrome [50]. Recently, a homozygous OTUD7A-L233F mutation was found in a patient with the 15q13.3 microdeletion syndrome with characterized proteasome dysfunction presumably caused by the loss of function of the OTUD7A deubiquitinase activity [51]. Although it remains unclear if these neurological disorders caused by OTUD7A dysfunction are limited to changes in dendritic spines, the results herein offer additional considerations if 7Ai or other OTUD7A inhibitors begin preclinical evaluation for Ewing sarcoma, including more in-depth investigations are warranted to further examine if OTUD7A also regulates neurological function in adult animals.

Notably, the effective dose of 7Ai in retarding Ewing sarcoma cell growth in vitro is about 10 μM (optionally about 1 μM to about 20 μM). Since standard first-line therapy for Ewing sarcoma includes cytotoxic chemotherapies, another option is to combine 7Ai with active chemotherapeutic drugs to achieve improved clinical outcome, including the development of metastasis. Because of the development of other biologically targeted therapies, including those directed at EWS-FLIT and USP19 and USP7, use of 7Ai with these agents is also disclosed herein.

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
1. Delattre O, Zucman J, Plougastel B, Desmaze C, Melot T, Peter M, Kovar H, Joubert I, de Jong P, Rouleau G, and et al., Gene fusion with an ETS DNA-binding domain caused by chromosome translocation in human tumours. Nature, 359(6391): p. 162-5.1992.
2. Patel M, Simon J M, Iglesia M D, Wu S B, McFadden A W, Lieb J D, and Davis I J, Tumor-specific retargeting of an oncogenic transcription factor chimera results in dysregulation of chromatin and transcription. Genome Res, 22(2): p. 259-70.2012.
3. Gangwal K, Sankar S, Hollenhorst P C, Kinsey M, Haroldsen S C, Shah A A, Boucher K M, Watkins W S, Jorde L B, Graves B J, and Lessnick S L, Microsatellites as EWS/FLI response elements in Ewing's sarcoma. Proc Natl Acad Sci USA, 105(29): p. 10149-54.2008.
4. Johnson K M, Mahler N R, Saund R S, Theisen E R, Taslim C, Callender N W, Crow J C, Miller K R, and Lessnick S L, Role for the EWS domain of EWS/FLI in binding GGAA-microsatellites required for Ewing sarcoma anchorage independent growth. Proc Natl Acad Sci USA, 114(37): p. 9870-9875.2017.
5. Boulay G, Sandoval G J, Riggi N, Iyer S, Buisson R, Naigles B, Awad M E, Rengarajan S, Volorio A, McBride M J, Broye L C, Zou L, Stamenkovic I, Kadoch C, and Rivera M N, Cancer-Specific Retargeting of BAF Complexes by a Prion-like Domain. Cell, 171(1): p. 163-178 e19.2017.
6. Yang L, Chansky H A, and Hickstein D D, EWS.Fli-1 fusion protein interacts with hyperphosphorylated RNA polymerase I I and interferes with serine-arginine protein-mediated RNA splicing. J Biol Chem, 275(48): p. 37612-8.2000.
7. Ramakrishnan R, Fujimura Y, Zou J P, Liu F, Lee L, Rao V N, and Reddy E S, Role of protein-protein interactions in the antiapoptotic function of EWS-Fli-1. Oncogene, 23(42): p. 7087-94.2004.
8. Toretsky J A, Erkizan V, Levenson A, Abaan O D, Parvin J D, Cripe T P, Rice A M, Lee S B, and Uren A, Oncoprotein EWS-FLI1 activity is enhanced by RNA helicase A. Cancer Res, 66(11): p. 5574-81.2006.
9. Cidre-Aranaz F and Alonso J, EWS/FLI1 Target Genes and Therapeutic Opportunities in Ewing Sarcoma. Front Oncol, 5: p. 162.2015.
10. Franzetti G A, Laud-Duval K, van der Ent W, Brisac A, Irondelle M, Aubert S, Dirksen U, Bouvier C, de Pinieux G, Snaar-Jagalska E, Chavrier P, and Delattre O, Cell-to-cell heterogeneity of EWSR1-FLI1 activity determines proliferation/migration choices in Ewing sarcoma cells. Oncogene, 36(25): p. 3505-3514.2017.
11. Katschnig A M, Kauer M O, Schwentner R, Tomazou E M, Mutz C N, Linder M, Sibilia M, Alonso J, Aryee D N T, and Kovar H, EWS-FLI1 perturbs MRTFB/YAP-1/TEAD target gene regulation inhibiting cytoskeletal autoregulatory feedback in Ewing sarcoma. Oncogene, 36(43): p. 5995-6005.2017.
12. Uren A and Toretsky J A, Ewing's sarcoma oncoprotein EWS-FLI1: the perfect target without a therapeutic agent. Future Oncol, 1(4): p. 521-8.2005.
13. Saravana P. Selvanathan E M, Garrett T. Graham, Katti Jessen, Brian Lannutti, Aykut Uren and Jeffrey A. Toretsky, Abstract 694: TK-216: a novel, first-in-class, small molecule inhibitor of EWS-FLI1 in early clinical development, for the treatment of Ewing Sarcoma. Cancer Research. 2017.
14. Caropreso V, Darvishi E, Turbyville T J, Ratnayake R, Grohar P J, McMahon J B, and Woldemichael G M, Englerin A Inhibits EWS-FLI1 DNA Binding in Ewing Sarcoma Cells. J Biol Chem, 291(19): p. 10058-66.2016.
15. Pattenden S G, Simon J M, Wali A, Jayakody C N, Troutman J, McFadden A W, Wooten J, Wood C C, Frye S V, Janzen W P, and Davis I J, High-throughput small molecule screen identifies inhibitors of aberrant chromatin accessibility. Proc Natl Acad Sci USA, 113(11): p. 3018-23.2016.
16. Gierisch M E, Pfistner F, Lopez-Garcia L A, Harder L, Schafer B W, and Niggli F K, Proteasomal Degradation of the EWS-FLI1 Fusion Protein Is Regulated by a Single Lysine Residue. J Biol Chem, 291(52): p. 26922-26933.2016.
17. Elzi D J, Song M, Hakala K, Weintraub S T, and Shiio Y, Proteomic Analysis of the EWS-Fli-1 Interactome Reveals the Role of the Lysosome in EWS-Fli-1 Turnover. J Proteome Res, 13(8): p. 3783-91.2014.
18. Stolte B, Iniguez A B, Dharia N V, Robichaud A L, Conway A S, Morgan A M, Alexe G, Schauer N J, Liu X, Bird G H, Tsherniak A, Vazquez F, Buhrlage S J, Walensky L D, and Stegmaier K, Genome-scale CRISPR-Cas9 screen identifies druggable dependencies in TP53 wild-type Ewing sarcoma. J Exp Med, 215(8): p. 2137-2155.2018.
19. Gorthi A, Romero J C, Loranc E, Cao L, Lawrence L A, Goodale E, Iniguez A B, Bernard X, Masamsetti V P, Roston S, Lawlor E R, Toretsky J A, Stegmaier K, Lessnick S L, Chen Y, and Bishop A J R, EWS-FLI1 increases transcription to cause R-loops and block BRCA1 repair in Ewing sarcoma. Nature, 555(7696): p. 387-391.2018.
20. Wang Z, Kang W, You Y, Pang J, Ren H, Suo Z, Liu H, and Zheng Y, USP7: Novel Drug Target in Cancer Therapy. Front Pharmacol, 10: p. 427.2019.
21. Coyne E S, Bedard N, Wykes L, Stretch C, Jammoul S, Li S, Zhang K, Sladek R S, Bathe O F, Jagoe R T, Posner B I, and Wing S S, Knockout of USP19 Deubiquitinating Enzyme Prevents Muscle Wasting by Modulating Insulin and Glucocorticoid Signaling. Endocrinology, 159(8): p. 2966-2977.2018.
22. Jin S, Tian S, Chen Y, Zhang C, Xie W, Xia X, Cui J, and Wang R F, USP19 modulates autophagy and antiviral immune responses by deubiquitinating Beclin-1. EMBO J, 35(8): p. 866-80.2016.
23. Altun M, Zhao B, Velasco K, Liu H, Hassink G, Paschke J, Pereira T, and Lindsten K, Ubiquitin-specific protease 19 (USP19) regulates hypoxia-inducible factor 1alpha (HIF-1alpha) during hypoxia. J Biol Chem, 287(3): p. 1962-9.2012.
24. Lei C Q, Wu X, Zhong X, Jiang L, Zhong B, and Shu H B, USP19 Inhibits TNF-alpha- and IL-1beta-Triggered N F-kappaB Activation by Deubiquitinating TAK1. J Immunol, 203(1): p. 259-268.2019.
25. Turnbull A P, Ioannidis S, Krajewski W W, Pinto-Fernandez A, Heride C, Martin A C L, Tonkin L M, Townsend E C, Buker S M, Lancia D R, Caravella J A, Toms A V, Charlton T M, Lahdenranta J, Wilker E, Follows B C, Evans N J, Stead L, Alli C, Zarayskiy V V, Talbot A C, Buckmelter A J, Wang M, McKinnon C L, Saab F, McGouran J F, Century H, Gersch M, Pittman M S, Marshall C G, Raynham T M, Simcox M, Stewart L M D, McLoughlin S B, Escobedo J A, Bair K W, Dinsmore C J, Hammonds T R, Kim S, Urbe S, Clague M J, Kessler B M, and Komander D, Molecular basis of USP7 inhibition by selective small-molecule inhibitors. Nature, 550(7677): p. 481-486.2017.
26. Kategaya L, Di Lello P, Rouge L, Pastor R, Clark K R, Drummond J, Kleinheinz T, Lin E, Upton J P, Prakash S, Heideker J, McCleland M, Ritorto M S, Alessi D R, Trost M, Bainbridge T W, Kwok M C M, Ma T P, Stiffler Z, Brasher B, Tang Y, Jaishankar P, Hearn B R, Renslo A R, Arkin M R, Cohen F, Yu K, Peale F, Gnad F, Chang M T, Klijn C, Blackwood E, Martin S E, Forrest W F, Ernst J A, Ndubaku C, Wang X, Beresini M H, Tsui V, Schwerdtfeger C, Blake R A, Murray J, Maurer T, and Wertz I E, USP7 small-molecule inhibitors interfere with ubiquitin binding. Nature, 550(7677): p. 534-538.2017.
27. Gerald Gavory C O D, Ewelina Rozycka, Anthony Dossang, Ashling Henderson, Caroline Hughes, Hugues Miel, Oliver Barker, Joana Costa, Peter Hewitt, Mary McFarland, Lauren Proctor and Tim Harrison, Abstract 1181: Discovery and development of novel highly potent and selective inhibitors of USP19 using UbiPlex™. Cancer Research. 2017.
28. May W A, Lessnick S L, Braun B S, Klemsz M, Lewis B C, Lunsford L B, Hromas R, and Denny C T, The Ewing's sarcoma EWS/FLI-1 fusion gene encodes a more potent transcriptional activator and is a more powerful transforming gene than FLI-1. Mol Cell Biol, 13(12): p. 7393-8.1993.
29. Dai X, Gan W, Li X, Wang S, Zhang W, Huang L, Liu S, Zhong Q, Guo J, Zhang J, Chen T, Shimizu K, Beca F, Blattner M, Vasudevan D, Buckley D L, Qi J, Buser L, Liu P, Inuzuka H, Beck A H, Wang L, Wild P J, Garraway L A, Rubin M A, Barbieri C E, Wong K K, Muthuswamy S K, Huang J, Chen Y, Bradner J E, and Wei W, Prostate cancer-associated SPOP mutations confer resistance to BET inhibitors through stabilization of BRD4. Nat Med, 23(9): p. 1063-1071.2017.
30. Zhang P, Wang D, Zhao Y, Ren S, Gao K, Ye Z, Wang S, Pan C W, Zhu Y, Yan Y, Yang Y, Wu D, He Y, Zhang J, Lu D, Liu X, Yu L, Zhao S, Li Y, Lin D, Wang Y, Wang L, Chen Y, Sun Y, Wang C, and Huang H, Intrinsic BET inhibitor resistance in SPOP-mutated prostate cancer is mediated by BET protein stabilization and AKT-mTORC1 activation. Nat Med, 23(9): p. 1055-1062.2017.
31. Janouskova H, El Tekle G, Bellini E, Udeshi N D, Rinaldi A, Ulbricht A, Bemasocchi T, Civenni G, Losa M, Svinkina T, Bielski C M, Kryukov G V, Cascione L, Napoli S, Enchev R I, Mutch D G, Carney M E, Berchuck A, Winterhoff B J N, Broaddus R R, Schraml P, Moch H, Bertoni F, Catapano C V, Peter M, Carr S A, Garraway L A, Wild P J, and Theurillat J P, Opposing effects of cancer-type-specific SPOP mutants on BET protein degradation and sensitivity to BET inhibitors. Nat Med, 23(9): p. 1046-1054.2017.
32. Gollavilli P N, Pawar A, Wilder-Romans K, Natesan R, Engelke C G, Dommeti V L, Krishnamurthy P M, Nallasivam A, Apel I J, Xu T, Qin Z S, Feng F Y, and Asangani I A, EWS/ETS-Driven Ewing Sarcoma Requires BET Bromodomain Proteins. Cancer Res, 78(16): p. 4760-4773.2018.
33. Gan W, Dai X, Lunardi A, Li Z, Inuzuka H, Liu P, Varmeh S, Zhang J, Cheng L, Sun Y, Asara J M, Beck A H, Huang J, Pandolfi P P, and Wei W, SPOP Promotes Ubiquitination and Degradation of the ERG Oncoprotein to Suppress Prostate Cancer Progression. Mol Cell, 59(6): p. 917-30.2015.
34. Kronke J, Fink E C, Hollenbach P W, MacBeth K J, Hurst S N, Udeshi N D, Chamberlain P P, Mani D R, Man H W, Gandhi A K, Svinkina T, Schneider R K, McConkey M, Jaras M, Griffiths E, Wetzler M, Bullinger L, Cathers B E, Carr S A, Chopra R, and Ebert B L, Lenalidomide induces ubiquitination and degradation of CK1alpha in del(5q) MDS. Nature, 523(7559): p. 183-188.2015.
35. Wimuttisuk W and Singer J D, The Cullin3 ubiquitin ligase functions as a Nedd8-bound heterodimer. Mol Biol Cell, 18(3): p. 899-909.2007.
36. Zhang L, Peng S, Dai X, Gan W, Nie X, Wei W, Hu G, and Guo J, Tumor suppressor SPOP ubiquitinates and degrades EglN2 to compromise growth of prostate cancer cells. Cancer Lett, 390: p. 11-20.2017.
37. Schittek B and Sinnberg T, Biological functions of casein kinase 1 isoforms and putative roles in tumorigenesis. Mol Cancer, 13: p. 231.2014.
38. Komander D, Clague M J, and Urbe S, Breaking the chains: structure and function of the deubiquitinases. Nat Rev Mol Cell Biol, 10(8): p. 550-63.2009.
39. Mevissen T E, Hospenthal M K, Geurink P P, Elliott P R, Akutsu M, Amaudo N, Ekkebus R, Kulathu Y, Wauer T, El Oualid F, Freund S M, Ovaa H, and Komander D, OTU deubiquitinases reveal mechanisms of linkage specificity and enable ubiquitin chain restriction analysis. Cell, 154(1): p. 169-84.2013.
40. Wang B, Jie Z, Joo D, Ordureau A, Liu P, Gan W, Guo J, Zhang J, North B J, Dai X, Cheng X, Bian X, Zhang L, Harper J W, Sun S C, and Wei W, TRAF2 and OTUD7B govern a ubiquitin-dependent switch that regulates mTORC2 signalling. Nature, 545(7654): p. 365-369.2017.
41. Hu H, Brittain G C, Chang J H, Puebla-Osorio N, Jin J, Zal A, Xiao Y, Cheng X, Chang M, Fu Y X, Zal T, Zhu C, and Sun S C, OTUD7B controls non-canonical NF-kappaB activation through deubiquitination of TRAF3. Nature, 494(7437): p. 371-4.2013.
42. Bonacci T, Suzuki A, Grant G D, Stanley N, Cook J G, Brown N G, and Emanuele M J, Cezanne/OTUD7B is a cell cycle-regulated deubiquitinase that antagonizes the degradation of APC/C substrates. EMBO J, 37(16). 2018.
43. Riggi N, Suva M L, Suva D, Cironi L, Provero P, Tercier S, Joseph J M, Stehle J C, Baumer K, Kindler V, and Stamenkovic I, EWS-FLI-1 expression triggers a Ewing's sarcoma initiation program in primary human mesenchymal stem cells. Cancer Res, 68(7): p. 2176-85.2008.
44. Hancock J D and Lessnick S L, A transcriptional profiling meta-analysis reveals a core EWS-FLI gene expression signature. Cell Cycle, 7(2): p. 250-6.2008.
45. Tanabe Y, Suehara Y, Kohsaka S, Hayashi T, Akaike K, Mukaihara K, Kurihara T, Kim Y, Okubo T, Ishii M, Kazuno S, Kaneko K, and Saito T, IRElalpha-XBP1 inhibitors exerted anti-tumor activities in Ewing's sarcoma. Oncotarget, 9(18): p. 14428-14443.2018.
46. Izhar Wallach M D, Abraham Heifets, AtomNet: A Deep Convolutional Neural Network for Bioactivity Prediction in Structure-based Drug Discovery. arXiv: p. 1510.02855.2015.
47. Hsieh C H, Li L, Vanhauwaert R, Nguyen K T, Davis M D, Bu G, Wszolek Z K, and Wang X, Mirol Marks Parkinson's Disease Subset and Mirol Reducer Rescues Neuron Loss in Parkinson's Models. Cell Metab, 30(6): p. 1131-1140 e7.2019.
48. Gierisch M E, Pedot G, Walser F, Lopez-Garcia L A, Jaaks P, Niggli F K, and Schafer B W, USP19 deubiquitinates EWS-FLI1 to regulate Ewing sarcoma growth. Sci Rep, 9(1): p. 951.2019.
49. Yin J, Chen W, Chao E S, Soriano S, Wang L, Wang W, Cummock S E, Tao H, Pang K, Liu Z, Pereira F A, Samaco R C, Zoghbi H Y, Xue M, and Schaaf C P, Otud7a Knockout Mice Recapitulate Many Neurological Features of 15q13.3 Microdeletion Syndrome. Am J Hum Genet, 102(2): p. 296-308.2018.
50. Uddin M, Unda B K, Kwan V, Holzapfel N T, White S H, Chalil L, Woodbury-Smith M, Ho K S, Harward E, Murtaza N, Dave B, Pellecchia G, D'Abate L, Nalpathamkalam T, Lamoureux S, Wei J, Speevak M, Stavropoulos J, Hope K J, Doble B W, Nielsen J, Wassman E R, Scherer S W, and Singh K K, OTUD7A Regulates Neurodevelopmental Phenotypes in the 15q13.3 Microdeletion Syndrome. Am J Hum Genet, 102(2): p. 278-295.2018.
51. Garret P, Ebstein F, Delplancq G, Dozieres-Puyravel B, Boughalem A, Auvin S, Duffourd Y, Klafack S, Zieba B A, Mahmoudi S, Singh K K, Duplomb L, Thauvin-Robinet C, Costa J M, Kruger E, Trost D, Verloes A, Faivre L, and Vitobello A, Report of the first patient with a homozygous OTUD7A variant responsible for epileptic encephalopathy and related proteasome dysfunction. Clin Genet, 97(4): p. 567-575.2020.
52. Liu P, Begley M, Michowski W, Inuzuka H, Ginzberg M, Gao D, Tsou P, Gan W, Papa A, Kim B M, Wan L, Singh A, Zhai B, Yuan M, Wang Z, Gygi S P, Lee T H, Lu K P, Toker A, Pandolfi P P, Asara J M, Kirschner M W, Sicinski P, Cantley L, and Wei W, Cell-cycle-regulated activation of Akt kinase by phosphorylation at its carboxyl terminus. Nature, 508(7497): p. 541-5.2014.
53. Liu P, Gan W, Chin Y R, Ogura K, Guo J, Zhang J, Wang B, Blenis J, Cantley L C, Toker A, Su B, and Wei W, PtdIns(3,4,5)P3-Dependent Activation of the mTORC2 Kinase Complex. Cancer Discov, 5(11): p. 1194-209.2015.
54. Jiang Y, Zhang Y, Leung J Y, Fan C, Popov K I, Su S, Qian J, Wang X, Holtzhausen A, Ubil E, Xiang Y, Davis I, Dokholyan N V, Wu G, Perou C M, Kim W Y, Earp H S, and Liu P, MERTK mediated novel site Akt phosphorylation alleviates SAV1 suppression. Nat Commun, 10(1): p. 1515.2019.
55. Liu P, Gan W, Guo C, Xie A, Gao D, Guo J, Zhang J, Willis N, Su A, Asara J M, Scully R, and Wei W, Akt-mediated phosphorylation of XLF impairs non-homologous end-joining DNA repair. Mol Cell, 57(4): p. 648-661.2015.
56. Guo J, Chakraborty A A, Liu P, Gan W, Zheng X, Inuzuka H, Wang B, Zhang J, Zhang L, Yuan M, Novak J, Cheng J Q, Toker A, Signoretti S, Zhang Q, Asara J M, Kaelin W G, Jr., and Wei W, pVHL suppresses kinase activity of Akt in a proline-hydroxylation-dependent manner. Science, 353(6302): p. 929-32.2016.
57. Liu P, Gan W, Su S, Hauenstein A V, Fu T M, Brasher B, Schwerdtfeger C, Liang A C, Xu M, and Wei W, K63-linked polyubiquitin chains bind to DNA to facilitate DNA damage repair. Sci Signal, 11(533). 2018.
58. Cong L, Ran F A, Cox D, Lin S, Barretto R, Habib N, Hsu P D, Wu X, Jiang W, Marraffini L A, and Zhang F, Multiplex genome engineering using CRISPR/Cas systems. Science, 339(6121): p. 819-23.2013.
59. Mertins P, Tang L C, Krug K, Clark D J, Gritsenko M A, Chen L, Clauser K R, Clauss T R, Shah P, Gillette M A, Petyuk V A, Thomas S N, Mani D R, Mundt F, Moore R J, Hu Y, Zhao R, Schnaubelt M, Keshishian H, Monroe M E, Zhang Z, Udeshi N D, Mani D, Davies S R, Townsend R R, Chan D W, Smith R D, Zhang H, Liu T, and Carr S A, Reproducible workflow for multiplexed deep-scale proteome and phosphoproteome analysis of tumor tissues by liquid chromatography-mass spectrometry. Nat Protoc, 13(7): p. 1632-1661.2018.
60. Zhang Y, Ma Z, Wang Y, Boyer J, Ni G, Cheng L, Su S, Zhang Z, Zhu Z, Qian J, Su L, Zhang Q, Damania B, and Liu P, Streptavidin Promotes DNA Binding and Activation of cGAS to Enhance Innate Immunity. iScience, 23(9): p. 101463.2020.
61. Mevissen T E T, Kulathu Y, Mulder M P C, Geurink P P, Maslen S L, Gersch M, Elliott P R, Burke J E, van Tol B D M, Akutsu M, Oualid F E, Kawasaki M, Freund S M V, Ovaa H, and Komander D, Molecular basis of Lys11-polyubiquitin specificity in the deubiquitinase Cezanne. Nature, 538(7625): p. 402-405.2016.
62. Hendlich M, Rippmann F, and Bamickel G, LIGSITE: automatic and efficient detection of potential small molecule-binding sites in proteins. J Mol Graph Model, 15(6): p. 359-63, 389.1997.
63. Diederik P. Kingma J B, Adam: A Method for Stochastic Optimization. arXiv: p. 1412.6980 2014.
64. Bruns R F and Watson I A, Rules for identifying potentially reactive or promiscuous compounds. J Med Chem, 55(22): p. 9763-72.2012.

It will be understood that various details of the presently disclosed subject matter may be changed without departing from the scope of the presently disclosed subject matter. Furthermore, the foregoing description is for the purpose of illustration only, and not for the purpose of limitation.

1. A composition for targeting OTUD7A, the composition comprising a component sufficient to block and/or reduce OTUD7A-mediated deubiquitination of EWS-FLI1 in a cell, wherein the component comprises 7Ai and variants thereof.

2. The composition of claim 1, wherein the component is a catalytic inhibitor, wherein the catalytic inhibitor inhibits the catalytic activity of OTUD7A.

3. The composition of claim 1, wherein blocking and/or reducing OTUD7A-mediated deubiquitination of EWS-FLI1 substantially destabilizes EWS-FLI1 in the cell, wherein the stability of EWS-FLI1 is reduced by about 50% or more.

4-6. (canceled)

7. The composition of claim 1, wherein 7Ai comprises the following structure:

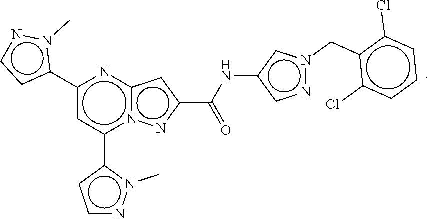

8. The composition of claim 1, wherein 7Ai is in the composition at a concentration sufficient to provide a dosage of about 10 μM when administered to a subject.

9. The composition of claim 1, wherein the composition comprises a delivery vehicle for the component, wherein the delivery vehicle is an expression vector, nanoparticle, liposome or vesicle.

10. (canceled)

11. An OTUD7A catalytic inhibitor, the catalytic inhibitor comprising a 7Ai compound or variant thereof.

12. The catalytic inhibitor of claim 11, wherein the catalytic inhibitor is configured to substantially limit Ewing sarcoma growth in vivo by degrading EWS-FLI1.

13. The catalytic inhibitor of claim 11, wherein the 7Ai compound comprises the following structure:

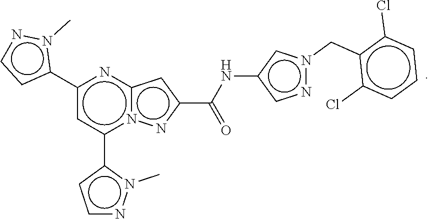

14. A method of treating Ewing sarcoma and related conditions, the method comprising administering to a subject having Ewing sarcoma or suspected of suffering from Ewing sarcoma a composition comprising a component targeting a fusion oncoprotein, wherein the fusion oncoprotein comprises EWS-FLIT, wherein the component comprises 7Ai or variant thereof.

15. The method of claim 14, wherein the component targets OTUD7A.

16. The method of claim 14, wherein the component blocks and or reduces OTUD7A-mediated deubiquitination of EWS-FLI1 in a cell in the subject.

17. The method of claim 14, wherein the component is a catalytic inhibitor, wherein the catalytic inhibitor inhibits the catalytic activity of OTUD7A.

18. The method of claim 14, wherein blocking and/or reducing OTUD7A-mediated deubiquitination of EWS-FLI1 substantially destabilizes EWS-FLI1, wherein the stability of EWS-FLI1 is reduced by about 50% or more.

19. The method of claim 14, wherein the component substantially reduces EWS-FLI1 levels in the cell.

20. The method of claim 14, wherein the 7Ai comprises the following structure:

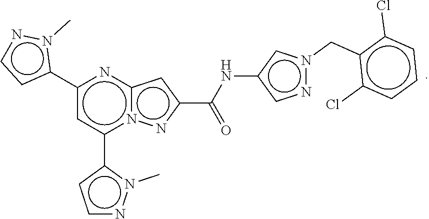

21. The method of claim 14, wherein the subject is suffering from a cancer or is believed to be suffering from a cancer, wherein the cancer is characterized by FLIT overexpression or dependent on FLI1 for proliferation.

22. The method of claim 14, wherein 7Ai is in the composition at a concentration sufficient to provide a dosage of about 10 μM when administered to a subject.

23. The method of claim 14, wherein the composition comprises a delivery vehicle for the component, wherein the delivery vehicle is an expression vector, nanoparticle, liposome or vesicle.

24. The method of claim 14, wherein the composition is co-administered to the subject with at least one chemotherapeutic drug.

25-32. (canceled)


SEQUENCE LISTING (103 sequences)

SEQ ID NO: 1 - protein, 5 aa, Homo sapiens
VTSSS

SEQ ID NO: 2 - DNA, 31 bp, artificial sequence
  synthetic sequence
gcatagatctgcgtccacggattacagtacc

SEQ ID NO: 3 - DNA, 30 bp, Artificial sequence
  synthetic sequence
gcatagatctgacgggactattaaggaggc

SEQ ID NO: 4 - DNA, 31 bp, Artificial sequence
  synthetic sequence
gcatctcgagctagtagtagctgcctaagtg

SEQ ID NO: 5 - DNA, 32 bp, Artificial sequence
  synthetic sequence
gcatggatcctcaagggttccaagtcctccac

SEQ ID NO: 6 - DNA, 34 bp, Artificial sequence
  synthetic sequence
gcatctcgagttaggattgcttcaggcgtttgcg

SEQ ID NO: 7 - DNA, 32 bp, Artificial sequence
  synthetic sequence
gcatagatctgtttctagtgtgcttccaaacc

SEQ ID NO: 8 - DNA, 26 bp, Artificial sequence
  synthetic sequence
gcatgtcgactcacagctcctcgcgg

SEQ ID NO: 9 - DNA, 45 bp, Artificial sequence
  synthetic sequence
cctccatgcctgtcactgccgccgccttctttggagccgcatcac

SEQ ID NO: 10 - DNA, 45 bp, Artificial sequence
  synthetic sequence
gtgatgcggctccaaagaaggcggcggcagtgacaggcatggagg

SEQ ID NO: 11 - DNA, 38 bp, Artificial sequence
  synthetic sequence
caggggatgggaactcccttttacatgctgcttcactg

SEQ ID NO: 12 - DNA, 38 bp, Artificial sequence
  synthetic sequence
cagtgaagcagcatgtaaaagggagttcccatcccctg

SEQ ID NO: 13 - DNA, 43 bp, Artificial
  synthetic sequence
gttcgatcgcttcctcctgcaatcattcaacaatgaccgtctc

SEQ ID NO: 14 - DNA, 43 bp, Artificial sequence
  synthetic sequence
gagacggtcattgttgaatgattgcaggaggaagcgatcgaac

SEQ ID NO: 15 - DNA, 22 bp, Artificial sequence
  synthetic sequence
tcctacagccaagctccaagtc

SEQ ID NO: 16 - DNA, 20 bp, Artificial sequence
  synthetic sequence
actccccgttggtcccctcc

SEQ ID NO: 17 - DNA, 20 bp, Artificial sequence
  synthetic sequence
cagtcactgcacctccatcc

SEQ ID NO: 18 - DNA, 20 bp, Artificial sequence
  synthetic sequence
ttcatgttattgccccaagc

SEQ ID NO: 19 - DNA, 20 bp, Artificial sequence
  synthetic sequence
ctacgacagcagcgacaacc

SEQ ID NO: 20 - DNA, 20 bp, Artificial sequence
  synthetic sequence
gccttggagaaaagcactcg

SEQ ID NO: 21 - DNA, 20 bp, Artificial sequence
  synthetic sequence
catctgtgagaagccacagg

SEQ ID NO: 22 - DNA, 21 bp, Artificial sequence
  synthetic sequence
tgcactcatcagagctacagg

SEQ ID NO: 23 - DNA, 20 bp, Artificial sequence
  synthetic sequence
ctgctcagatttccccaaag

SEQ ID NO: 24 - DNA, 20 bp, Artificial sequence
  synthetic sequence
tggcatcaagcaggtcatag

SEQ ID NO: 25 - DNA, 21 bp, Artificial sequence
  synthetic sequence
catcaagaaagggcatgctaa

SEQ ID NO: 26 - DNA, 21 bp, Artificial sequence
  synthetic sequence
ctacggcagggaccatattct

SEQ ID NO: 27 - DNA, 22 bp, Artificial sequence
  synthetic sequence
caggtctcccacaaacacatcg

SEQ ID NO: 28 - DNA, 25 bp, Artificial sequence
  synthetic sequence
accaggtctttatcctccaagtagc

SEQ ID NO: 29 - DNA, 22 bp, Artificial sequence
  synthetic sequence
cgcacgatttcattgaacactt

SEQ ID NO: 30 - DNA, 21 bp, Artificial sequence
  synthetic sequence
cggattggaaatacttcacat

SEQ ID NO: 31 - DNA, 27 bp, Artificial sequence
  synthetic sequence
cctctgtgctacagattatacctttgc

SEQ ID NO: 32 - DNA, 18 bp, Artificial sequence
  synthetic sequence
ttgcactgcagccccaat

SEQ ID NO: 33 - DNA, 22 bp, Artificial sequence
  synthetic sequence
tggagaaccaggctatggacgt

SEQ ID NO: 34 - DNA, 22 bp, Artificial sequence
  synthetic sequence
ccaggaactgtgagaagtgctg

SEQ ID NO: 35 - DNA, 21 bp, Artificial sequence
  synthetic sequence
ctgcggaaagtgctcatcagt

SEQ ID NO: 36 - DNA, 21 bp, Artificial sequence
  synthetic sequence
tggcagagcgaacaataaggc

SEQ ID NO: 37 - DNA, 24 bp, Artificial sequence
  synthetic sequence
gatgctgtgtgttttgatgttgac

SEQ ID NO: 38 - DNA, 24 bp, Artificial sequence
  synthetic sequence
cttgacttgttgcctgatcacatt

SEQ ID NO: 39 - DNA, 21 bp, Artificial sequence
  synthetic sequence
cttggtagtgtttgccatcgt

SEQ ID NO: 40 - DNA, 20 bp, Artificial sequence
  synthetic sequence
tgcgggtgaagaggaagtag

SEQ ID NO: 41 - DNA, 21 bp, Artificial sequence
  synthetic sequence
gagctctgaagargcatggag

SEQ ID NO: 42 - DNA, 20 bp, Artificial sequence
  synthetic sequence
tgcttctggaggttacagca

SEQ ID NO: 43 - DNA, 59 bp, Artificial sequence
  synthetic sequence
ccggcacagatcaaggtagtgaaatctcgagatttcactaccttgatctgtgttttttg

SEQ ID NO: 44 - DNA, 59 bp, Artificial sequence
  synthetic sequence
ccggcaaggtagtgaaattctcctactcgagtaggagaatttcactaccttgttttttg

SEQ ID NO: 45 - DNA, 59 bp, Artificial sequence
  synthetic sequence
ccggcaaacgcctgaagcaatcctactcgagtaggattgcttcaggcgtttgttttttg

SEQ ID NO: 46 - DNA, 59 bp, Artificial sequence
  synthetic sequence
ccggctcctacatgtggaccatcaactcgagttgatggtccacatgtaggagttttttg

SEQ ID NO: 47 - DNA, 58 bp, Artificial sequence
  synthetic sequence
ccggcgtgtgccaaatggtttgaaactcgagtttcaaaccatttggcacacgtttttg

SEQ ID NO: 48 - DNA, 58 bp, Artificial sequence
  synthetic sequence
ccggttcaggctttacaacgtttatctcgagataaacgttgtaaagcctgaatttttg

SEQ ID NO: 49 - DNA, 58 bp, Artificial sequence; identical to SEQ ID NO: 47
  synthetic sequence

SEQ ID NO: 50 - DNA, 57 bp, Artificial sequence
  synthetic sequence
ccggaggagtatgctgaagatgacactcgagtgtcatcttcagcatactcctttttt

SEQ ID NO: 51 - DNA, 57 bp, Artificial sequence
  synthetic sequence
ccggtgtttctatcgggctttcggactcgagtccgaaagcccgatagaaacattttt

SEQ ID NO: 52 - DNA, 57 bp, Artificial sequence
  synthetic sequence
ccggtgtggttgtaaatggtcctatctcgagataggaccatttacaaccacattttt

SEQ ID NO: 53 - DNA, 58 bp, Artificial sequence
  synthetic sequence
ccggcctatgtgtcactggattattctcgagaataatccagtgacacataggtttttg

SEQ ID NO: 54 - DNA, 57 bp, Artificial sequence
  synthetic sequence
ccggtgggctgctatgtctctgtatctcgagatacagagacatagcagcccattttt

SEQ ID NO: 55 - DNA, 57 bp, Artificial sequence
  synthetic sequence
ccggccttccgtttacctgctctatctcgagatagagcaggtaaacggaaggttttt

SEQ ID NO: 56 - DNA, 58 bp, Artificial sequence
  synthetic sequence
ccgggacgtctgccatcgcatattactcgagtaatatgcgatggcagacgtctttttg

SEQ ID NO: 57 - DNA, 58 bp, Artificial sequence
  synthetic sequence
ccggtttggaaatcagggcttaaatctcgagatttaagccctgatttccaaatttttg

SEQ ID NO: 58 - DNA, 58 bp, Artificial sequence
  synthetic sequence
ccgggggagttacacatcgcatatcctcgaggatatgcgatgtgtaactccctttttg

SEQ ID NO: 59 - DNA, 58 bp, Artificial sequence
  synthetic sequence
ccggcaagtcgagaatctaactattctcgagaatagttagattctcgacttgtttttg

SEQ ID NO: 60 - DNA, 58 bp, Artificial sequence
  synthetic sequence
ccggtatgcaatgccttagtcataactcgagttatgactaaggcattgcatatttttg

SEQ ID NO: 61 - DNA, 58 bp, Artificial sequence
  synthetic sequence
ccggcactatagattccaaacataactcgagttatgtttggaatctatagtgtttttg

SEQ ID NO: 62 - DNA, 59 bp, Artificial sequence
  synthetic sequence
ccggccatcattcaaaccagggtttctcgagaaaccctggtttgaatgatggttttttg

SEQ ID NO: 63 - DNA, 58 bp, Artificial sequence
  synthetic sequence
ccggccgactacttctccaactatgctcgagcatagttggagaagtagtcggtttttg

SEQ ID NO: 64 - DNA, 58 bp, Artificial sequence
  synthetic sequence
ccggagaacgtctgagccttcaatgctcgagcattgaaggctcagacgttcttttttg

SEQ ID NO: 65 - DNA, 59 bp, Artificial sequence
  synthetic sequence
ccggcatgatctactgcgacaacatctcgagatgttgtcgcagtagatcatgttttttg

SEQ ID NO: 66 - DNA, 59 bp, Artificial sequence
  synthetic sequence
ccggcaccaactaagatttggtcatctcgagatgaccaaatcttagttggtgttttttg

SEQ ID NO: 67 - DNA, 59 bp, Artificial sequence
  synthetic sequence
ccgggatttggtcatgttgcgtatactcgagtatacgcaacatgaccaaatcttttttg

SEQ ID NO: 68 - DNA, 59 bp, Artificial sequence
  synthetic sequence
ccgggcaaagctactaacaggtgttctcgagaacacctgttagtagctttgcttttttg

SEQ ID NO: 69 - DNA, 59 bp, Artificial sequence
  synthetic sequence
ccgggctgactactaaggagaataactcgagttattctccttagtagtcagcttttttg

SEQ ID NO: 70 - DNA, 59 bp, Artificial sequence
  synthetic sequence
ccggcgatgagactaatgcagtgaactcgagttcactgcattagtctcatcgttttttg

SEQ ID NO: 71 - DNA, 57 bp, Artificial sequence
  synthetic sequence
cgggcagcaattctaacagcaatactcgagtattgctgttagaattgctgctttttg

SEQ ID NO: 72 - DNA, 58 bp, Artificial sequence
  synthetic sequence
ccggcgcacacacttcagcaagaatctcgagattcttgctgaagtgtgtgcgtttttg

SEQ ID NO: 73 - DNA, 58 bp, Artificial sequence
  synthetic sequence
ccgggcgcgagaactgtgcgttctactcgagtagaacgcacagttctcgcgctttttg

SEQ ID NO: 74 - DNA, 62 bp, Artificial sequence
  synthetic sequence
gtaccggttgaagagtttcacgtctttgctcgagcaaagacgtgaaactcttcaattttttg

SEQ ID NO: 75 - DNA, 58 bp, Artificial sequence
  synthetic sequence
ccggtggaaatgctcacggtttatactcgagtataaaccgtgagcatttccatttttg

SEQ ID NO: 76 - DNA, 57 bp, Artificial sequence
  synthetic sequence
ccgggcaaggaggctaaacaaagttctcgagaactttgtttagcctccttgcttttt

SEQ ID NO: 77 - DNA, 57 bp, Artificial sequence
  synthetic sequence
ccgggcagaatttgcgatgtacttactcgagtaagtacatcgcaaattctgcttttt

SEQ ID NO: 78 - DNA, 59 bp, Artificial sequence
  synthetic sequence
ccgggcaagctctataagattcttcctcgaggaagaatcttatagagcttgcttttttg

SEQ ID NO: 79 - DNA, 55 bp, Artificial sequence
  synthetic sequence
tgcccatcctgcacacttacttcaagagagtaagtgtgcaggatgggcttttttc

SEQ ID NO: 80 - DNA, 57 bp, Artificial sequence
  synthetic sequence
ccgggcgcgagaactgtgcgttctactcgagtagaacgcacagttctcgcgcttttt

SEQ ID NO: 81 - DNA, 57 bp, Artificial sequence
  synthetic sequence
aattaaaaagcgcgagaactgtgcgttctactcgagtagaacgcacagttctcgcgc

SEQ ID NO: 82 - DNA, 30 bp, Artificial sequence
  synthetic sequence
ctgccagcgggaaaattgcgcgttctacgg

SEQ ID NO: 83 - DNA, 30 bp, Artificial sequence
  synthetic sequence
ccgtagaacgcgcaattttcccgctggcag

SEQ ID NO: 84 - DNA, 25 bp, Artificial sequence
  synthetic sequence
caccgtgcggctccaaagaagctgg

SEQ ID NO: 85 - DNA, 25 bp, Artificial sequence
  synthetic sequence
aaacccagcttctttggagccgcac

SEQ ID NO: 86 - DNA, 120 bp, Artificial sequence
  synthetic sequence
gcccaccagcagaaggtgaactttgtccctccccatccatcctccatgcctgtcactgccgccgccttctttggagccgcatcacaatactggacctcccccacggggggaatctacccc

SEQ ID NO: 87 - DNA, 24 bp, Artificial sequence
  synthetic sequence
gtgcacggcaaaagatatgcttac

SEQ ID NO: 88 - DNA, 23 bp, Artificial sequence
  synthetic sequence
ctagtagtagctgcctaagtgtg

SEQ ID NO: 89 - DNA, 24 bp, Artificial sequence
  synthetic sequence
caccgagacttgttcggtccacgg

SEQ ID NO: 90 - DNA, 25 bp, Artificial sequence
  synthetic sequence
aaacccgtccaccgaacaaagtctc

SEQ ID NO: 91 - DNA, 25 bp, Artificial sequence
  synthetic sequence
caccgtgctgcccaacactcagccg

SEQ ID NO: 92 - DNA, 24 bp, Artificial sequence
  synthetic sequence
aaaccggctgagtgttgggagcac

SEQ ID NO: 93 - DNA, 24 bp, Artificial sequence
  synthetic sequence
cacgcagaccaggttctgcccccg

SEQ ID NO: 94 - DNA, 25 bp, Artificial sequence
  synthetic sequence
aaaccgggggcagaacctggtctgc

SEQ ID NO: 95 - protein, 9 aa, Homo sapiens
MPVTSSSFF

SEQ ID NO: 96 - protein, 9 aa, Homo sapiens
MTASSSSDY

SEQ ID NO: 97 - DNA, 8 bp, Homo sapiens
  misc_feature (8)..(8): n is a, c, g, t or u
gmvsstsn

SEQ ID NO: 98 - protein, 9 aa, Homo sapiens
PVADSSTRV

SEQ ID NO: 99 - protein, 9 aa, Homo sapiens
GEASSTTSP

SEQ ID NO: 100 - protein, 9 aa, Homo sapiens
VSEGSSSGR

SEQ ID NO: 101 - protein, 9 aa, Homo sapiens
EQPSSTSGG

SEQ ID NO: 102 - protein, 9 aa, Homo sapiens
PDVSSSTHP

SEQ ID NO: 103 - protein, 9 aa, Homo sapiens
MPVTAAAFF